SEQUENCE LISTING (11 sequences)

SEQ ID NO: 1 - RNA, 1626 nt, synthetic construct
  source 1..1626: mol_type = other RNA
atgtgccaccagcagctggtcatcagctggttcagcctcgttttcctcgcctcgccgctggtcgccatatgggagctcaagaaggacgtatacgtggtggagctggactggtaccccgacgcgccgggcgagatggtcgtcctgacgtgcgacacgccggaggaggacggcatcacgtggacgctggaccagtccagcgaggtcctcggctccggcaagacgctgacgatccaggtcaaggagttcggcgacgcgggccagtacacgtgccacaagggcggcgaggtcctgagccactccctcctcctgctacacaagaaggaggacgggatctggagcacggacatcctcaaggaccagaaggagccgaagaacaagaccttcctgcgctgcgaggcgaagaattactcgggccggttcacgtgctggtggctcaccacgatcagcacggacctgacgttctcggtcaagtcgtcgcggggctcgtcggacccccagggggtgacctgcggcgcggcgacgctgtcggcggagcgggtgcggggcgacaacaaggagtacgagtactcggtcgagtgccaggaggactcggcgtgcccggcggcggaggagtcgctgccgatcgaggtgatggtcgacgcggtccacaagctgaagtacgagaactacacgtcgtcgttcttcatccgggacatcatcaagccggacccgccgaagaacctgcagctgaagccgctgaagaactcgcggcaggtcgaggtctcgtgggagtacccggacacgtggtcgacgccgcactcgtacttctcgctgacgttctgcgtccaagtgcagggcaagtcgaagcgggagaagaaggaccgggtgttcaccgacaagacgagcgcgacggtgatctgccggaagaacgcgtcgatctcggtgcgggcgcaggaccggtactactcgtcgtcgtggtcggagtgggcgtcggtgccgtgcagcggcggaagcggcggcggcagcggcggaggctctggcggcggctcccggaacctgccggtggcgacgccggacccggggatgttcccgtgcctgcaccacagccagaacctgctgcgggcggtgtcgaacatgctgcagaaggcgcggcagacgctggagttctacccgtgcacgagcgaggagatcgaccacgaggacatcacgaaggacaagaccagcacggtggaggcgtgcctgccgctggagctgacgaagaacgagtcgtgcctgaactcgagggagacgtcgttcatcacgaacgggtcgtgcctggcgtcgcggaagacgtcgttcatgatggcgctgtgcctgtcgtcgatctacgaggacctgaagatgtaccaggtggagttcaagacgatgaacgcgaagctgctgatggacccgaagcggcagatcttcctcgaccagaacatgctggcggtgatcgacgagctcatgcaggcgctcaacttcaacagcgagacggtgccgcagaagtcgtcgctcgaggagccggacttctacaagacgaagatcaagctctgcatcctgctgcacgctttccggatccgggcggtgacgatcgaccgggtgatgtcgtacctgaacgcttcgtgataa

SEQ ID NO: 2 - RNA, 987 nt, synthetic construct
  source 1..987: mol_type = other RNA
atgtgccaccagcagctggtcatcagctggttcagcctcgttttcctcgcctcgccgctggtcgccatatgggagctcaagaaggacgtatacgtggtggagctggactggtaccccgacgcgccgggcgagatggtcgtcctgacgtgcgacacgccggaggaggacggcatcacgtggacgctggaccagtccagcgaggtcctcggctccggcaagacgctgacgatccaggtcaaggagttcggcgacgcgggccagtacacgtgccacaagggcggcgaggtcctgagccactccctcctcctgctacacaagaaggaggacgggatctggagcacggacatcctcaaggaccagaaggagccgaagaacaagaccttcctgcgctgcgaggcgaagaattactcgggccggttcacgtgctggtggctcaccacgatcagcacggacctgacgttctcggtcaagtcgtcgcggggctcgtcggacccccagggggtgacctgcggcgcggcgacgctgtcggcggagcgggtgcggggcgacaacaaggagtacgagtactcggtcgagtgccaggaggactcggcgtgcccggcggcggaggagtcgctgccgatcgaggtgatggtcgacgcggtccacaagctgaagtacgagaactacacgtcgtcgttcttcatccgggacatcatcaagccggacccgccgaagaacctgcagctgaagccgctgaagaactcgcggcaggtcgaggtctcgtgggagtacccggacacgtggtcgacgccgcactcgtacttctcgctgacgttctgcgtccaagtgcagggcaagtcgaagcgggagaagaaggaccgggtgttcaccgacaagacgagcgcgacggtgatctgccggaagaacgcgtcgatctcggtgcgggcgcaggaccggtactactcgtcgtcgtggtcggagtgggcgtcggtgccgtgcagcggc

SEQ ID NO: 3 - RNA, 597 nt, synthetic construct
  source 1..597: mol_type = other RNA
cggaacctgccggtggcgacgccggacccggggatgttcccgtgcctgcaccacagccagaacctgctgcgggcggtgtcgaacatgctgcagaaggcgcggcagacgctggagttctacccgtgcacgagcgaggagatcgaccacgaggacatcacgaaggacaagaccagcacggtggaggcgtgcctgccgctggagctgacgaagaacgagtcgtgcctgaactcgagggagacgtcgttcatcacgaacgggtcgtgcctggcgtcgcggaagacgtcgttcatgatggcgctgtgcctgtcgtcgatctacgaggacctgaagatgtaccaggtggagttcaagacgatgaacgcgaagctgctgatggacccgaagcggcagatcttcctcgaccagaacatgctggcggtgatcgacgagctcatgcaggcgctcaacttcaacagcgagacggtgccgcagaagtcgtcgctcgaggagccggacttctacaagacgaagatcaagctctgcatcctgctgcacgctttccggatccgggcggtgacgatcgaccgggtgatgtcgtacctgaacgcttcgtgataa

SEQ ID NO: 4 - RNA, 42 nt, synthetic construct
  source 1..42: mol_type = other RNA
ggaagcggcggcggcagcggcggaggctctggcggcggctcc

SEQ ID NO: 5 - RNA, 7461 nt, synthetic construct
  source 1..7461: mol_type = other RNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcc

SEQ ID NO: 6 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 329 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSG

SEQ ID NO: 9 - protein, 14 aa, synthetic construct
GSGGGSGGGSGGGS

SEQ ID NO: 10 - DNA, 11259 bp, synthetic construct
  source 1..11259: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtgccaccagcagctggtcatcagctggttcagcctcgttttcctcgcctcgccgctggtcgccatatgggagctcaagaaggacgtatacgtggtggagctggactggtaccccgacgcgccgggcgagatggtcgtcctgacgtgcgacacgccggaggaggacggcatcacgtggacgctggaccagtccagcgaggtcctcggctccggcaagacgctgacgatccaggtcaaggagttcggcgacgcgggccagtacacgtgccacaagggcggcgaggtcctgagccactccctcctcctgctacacaagaaggaggacgggatctggagcacggacatcctcaaggaccagaaggagccgaagaacaagaccttcctgcgctgcgaggcgaagaattactcgggccggttcacgtgctggtggctcaccacgatcagcacggacctgacgttctcggtcaagtcgtcgcggggctcgtcggacccccagggggtgacctgcggcgcggcgacgctgtcggcggagcgggtgcggggcgacaacaaggagtacgagtactcggtcgagtgccaggaggactcggcgtgcccggcggcggaggagtcgctgccgatcgaggtgatggtcgacgcggtccacaagctgaagtacgagaactacacgtcgtcgttcttcatccgggacatcatcaagccggacccgccgaagaacctgcagctgaagccgctgaagaactcgcggcaggtcgaggtctcgtgggagtacccggacacgtggtcgacgccgcactcgtacttctcgctgacgttctgcgtccaagtgcagggcaagtcgaagcgggagaagaaggaccgggtgttcaccgacaagacgagcgcgacggtgatctgccggaagaacgcgtcgatctcggtgcgggcgcaggaccggtactactcgtcgtcgtggtcggagtgggcgtcggtgccgtgcagcggcggaagcggcggcggcagcggcggaggctctggcggcggctcccggaacctgccggtggcgacgccggacccggggatgttcccgtgcctgcaccacagccagaacctgctgcgggcggtgtcgaacatgctgcagaaggcgcggcagacgctggagttctacccgtgcacgagcgaggagatcgaccacgaggacatcacgaaggacaagaccagcacggtggaggcgtgcctgccgctggagctgacgaagaacgagtcgtgcctgaactcgagggagacgtcgttcatcacgaacgggtcgtgcctggcgtcgcggaagacgtcgttcatgatggcgctgtgcctgtcgtcgatctacgaggacctgaagatgtaccaggtggagttcaagacgatgaacgcgaagctgctgatggacccgaagcggcagatcttcctcgaccagaacatgctggcggtgatcgacgagctcatgcaggcgctcaacttcaacagcgagacggtgccgcagaagtcgtcgctcgaggagccggacttctacaagacgaagatcaagctctgcatcctgctgcacgctttccggatccgggcggtgacgatcgaccgggtgatgtcgtacctgaacgcttcgtgataaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaagaagagctagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctagggataacagggtaattaatacgactcactatag

SEQ ID NO: 11 - DNA, 11415 bp, synthetic construct
  source 1..11415: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtgccaccagcagctggtcatcagctggttcagcctcgttttcctcgcctcgccgctggtcgccatatgggagctcaagaaggacgtatacgtggtggagctggactggtaccccgacgcgccgggcgagatggtcgtcctgacgtgcgacacgccggaggaggacggcatcacgtggacgctggaccagtccagcgaggtcctcggctccggcaagacgctgacgatccaggtcaaggagttcggcgacgcgggccagtacacgtgccacaagggcggcgaggtcctgagccactccctcctcctgctacacaagaaggaggacgggatctggagcacggacatcctcaaggaccagaaggagccgaagaacaagaccttcctgcgctgcgaggcgaagaattactcgggccggttcacgtgctggtggctcaccacgatcagcacggacctgacgttctcggtcaagtcgtcgcggggctcgtcggacccccagggggtgacctgcggcgcggcgacgctgtcggcggagcgggtgcggggcgacaacaaggagtacgagtactcggtcgagtgccaggaggactcggcgtgcccggcggcggaggagtcgctgccgatcgaggtgatggtcgacgcggtccacaagctgaagtacgagaactacacgtcgtcgttcttcatccgggacatcatcaagccggacccgccgaagaacctgcagctgaagccgctgaagaactcgcggcaggtcgaggtctcgtgggagtacccggacacgtggtcgacgccgcactcgtacttctcgctgacgttctgcgtccaagtgcagggcaagtcgaagcgggagaagaaggaccgggtgttcaccgacaagacgagcgcgacggtgatctgccggaagaacgcgtcgatctcggtgcgggcgcaggaccggtactactcgtcgtcgtggtcggagtgggcgtcggtgccgtgcagcggcggaagcggcggcggcagcggcggaggctctggcggcggctcccggaacctgccggtggcgacgccggacccggggatgttcccgtgcctgcaccacagccagaacctgctgcgggcggtgtcgaacatgctgcagaaggcgcggcagacgctggagttctacccgtgcacgagcgaggagatcgaccacgaggacatcacgaaggacaagaccagcacggtggaggcgtgcctgccgctggagctgacgaagaacgagtcgtgcctgaactcgagggagacgtcgttcatcacgaacgggtcgtgcctggcgtcgcggaagacgtcgttcatgatggcgctgtgcctgtcgtcgatctacgaggacctgaagatgtaccaggtggagttcaagacgatgaacgcgaagctgctgatggacccgaagcggcagatcttcctcgaccagaacatgctggcggtgatcgacgagctcatgcaggcgctcaacttcaacagcgagacggtgccgcagaagtcgtcgctcgaggagccggacttctacaagacgaagatcaagctctgcatcctgctgcacgctttccggatccgggcggtgacgatcgaccgggtgatgtcgtacctgaacgcttcgtgataaggcgcgcccacccagcggccgctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaagaagagctagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgttaatacgactcactatagg

(19) United States
(12) Patent Application Publication
(10) Pub. No.: US 2024/0366740 A1
(43) Pub. Date: Nov. 7, 2024

(54) INTERLEUKIN-12 SELF-REPLICATING RNA AND METHODS

(71) Applicant: Immorna (Hangzhou) Biotechnology Co., Ltd., Hangzhou (CN)

(72) Inventors: Zihao Wang, Hangzhou (CN); Yuanqing Liu, Hangzhou (CN); Yanni Chen, Hangzhou (CN); Zhijun Guo, Hangzhou (CN)

(21) Appl. No.: 18/667,939

(22) Filed: May 17, 2024

Related U.S. Application Data
(63) Continuation of application No. PCT/CN2022/139738, filed on Dec. 16, 2022.

(51) Int. Cl. A61K 39/00; A61K 9/127; A61K 9/51; A61K 45/06; A61P 35/00; A61P 37/04

(52) U.S. Cl. CPC A61K 39/00114; A61K 9/1271; A61K 9/5123; A61K 45/06; A61P 35/00; A61P 37/04; A61K 2039/53; A61K 2039/55555

(57) ABSTRACT

The disclosure relates to a liposome packaged RNA replicon encoding IL-12. Included are methods for preparing and administering the liposome packaged RNA replicon encoding IL-12.

### CROSS-REFERENCE TO RELATED APPLICATIONS

This application is a continuation of International Application Number PCT/CN2022/139738 filed on Dec. 16, 2022, which claims the benefit of and priority to PCT/CN2021/139264, filed on Dec. 17, 2021, the entire disclosures of which are incorporated herein by reference for all purposes.

### REFERENCE TO A SEQUENCE LISTING

The present application contains a Sequence Listing which has been submitted electronically in XML format. The Sequence Listing XML is hereby incorporated by reference in its entirety. Said XML file, created on Dec. 16, 2022, is named IMO-002WO2-SL-as filed.xml and is 49,152 bytes in size.

### BACKGROUND

Interleukin-12 (IL-12) is a pro-inflammatory cytokine that plays an important role in innate and adaptive immunity. Gately, M K et al., Annu Rev Immunol. 16: 495-521 (1998). IL-12 functions primarily as a 70 kDa heterodimeric protein consisting of two disulfide-linked p35 and p40 subunits. IL-12 p40 homodimers do exist, but other than functioning as an antagonist that binds the IL-12 receptor, they do not appear to mediate a biologic response. The precursor form of the IL-12 p40 subunit (NM_002187; P29460; also referred to as IL-12B, natural killer cell stimulatory factor 2, cytotoxic lymphocyte maturation factor 2) is 328 amino acids in length, while its mature form is 306 amino acids long. The precursor form of the IL12 p35 subunit (NM_000882; P29459; also referred to as IL-12A, natural killer cell stimulatory factor 1, cytotoxic lymphocyte maturation factor 1) is 219 amino acids in length and the mature form is 197 amino acids long. The genes for the IL-12 p35 and p40 subunits reside on different chromosomes and are regulated independently of each other. Gately, M K et al., Annu Rev Immunol. 16: 495-521 (1998). Many different immune cells (e.g., dendritic cells, macrophages, monocytes, neutrophils, and B cells) produce IL-12 upon antigenic stimuli. The active IL-12 heterodimer is formed following protein synthesis.

Due to its ability to activate both NK cells and cytotoxic T cells, IL12 protein has been studied as a promising anti-cancer therapeutic since 1994. See Nastala, C. L. et al., J Immunol 153: 1697-1706 (1994). Despite high expectations, early clinical studies did not yield satisfactory results. Lasek W. et al., Cancer Immunol Immunother 63: 419-435, 424 (2014). Repeated administration of IL-12, in most patients, led to adaptive response and a progressive decline of IL-12-induced interferon gamma (IFN-7) levels in blood. Moreover, while it was recognized that IL-12-induced anti-cancer activity is largely mediated by the secondary secretion of IFN-7, the concomitant induction of IFN-γ along with other cytokines (e.g., TNF-α) or chemokines (IP-10 or MIG) by IL-12 caused severe toxicity.

In addition to the negative feedback and toxicity, the marginal efficacy of the IL-12 therapy in clinical settings may be caused by the strong immunosuppressive environment in humans. To minimize IFN-7 toxicity and improve IL-12 efficacy, scientists tried different approaches, such as different dose and time protocols for IL-12 therapy. See Sacco, S. et al., Blood 90: 4473-4479 (1997); Leonard, J. P. et al., Blood 90: 2541-2548 (1997); Coughlin, C. M. et al., Cancer Res. 57: 2460-2467 (1997); Asselin-Paturel, C. et al., Cancer 91: 113-122 (2001); and Saudemont, A. et al., Leukemia 16: 1637-1644 (2002). Nonetheless, these approaches have not significantly impacted patient survival. Kang, W. K., et al., Human Gene Therapy 12: 671-684 (2001).

Currently, a number of IL-12 clinical trials are on-going. Though these multiple clinical trials have been on-going for nearly 20 years since the first human clinical trial of IL-12 in 1996, an FDA-approved IL-12 product is still not available. Thus, there is a need in the art for an improved therapeutic approach for using IL-12 to treat tumors.

Described herein is a new modality, liposome packaged RNA replicon encoding IL-12, and, when delivered in vivo, triggers strong immune and T cell response towards tumor elimination. RNA replicon is capable of self-amplification within the transduced cells. The self-amplification leads to more RNA copies and subsequently long IL-12 expression time and high localized pro-inflammatory IL-12 concentration. The self-amplification process itself stimulates an innate immunity response that is linked to antitumor effects. This disclosure presents using biodegradable ionizable cationic lipids in its liposome formulation and hence less potential adverse events when administered to patients due to toxicity of the lipids.

### SUMMARY

Described herein is a self-replicating RNA (srRNA) comprising a nucleotide sequence encoding interleukin-12 (IL-12) comprising p40 and p35.

In some embodiments, p40 and p35 are operably linked.

In some embodiments, p40 and p35 are directly linked.

In some embodiments, p40 and p35 are linked via a cleavable linker.

In some embodiments, the interleukin-12 comprises human interleukin-12.

In some embodiments, the interleukin-12 comprises human p40 and human p35.

In some embodiments, p40 and p35 are operably linked.

In some embodiments, p40 and p35 are directly linked.

In some embodiments, p40 and p35 are linked via a cleavable linker.

In some embodiments, interleukin-12 comprises one or more sequences comprising the sequences shown in SEQ ID NOs: 1.

In some embodiments, the interleukin-12 comprises one or more sequences consisting of the sequences shown in SEQ ID NOs: 1.

In some embodiments, the sequence of p35 comprises the sequence shown in SEQ ID NO: 3.

In some embodiments, the sequence of p35 consists of the sequence shown in SEQ ID NO: 3.

In some embodiments, the sequence of p40 comprises the sequence shown in SEQ ID NO: 2.

In some embodiments, the sequence of p40 consists of the sequence shown in SEQ ID NO: 2.

In some embodiments, the sequence of the linker comprises the sequence shown in SEQ ID NO: 4.

In some embodiments, the sequence of the linker consists of the sequence shown in SEQ ID NO: 4.

In some embodiments, the nucleotide sequence encoding interleukin-12 is operably linked to a promoter, optionally a subgenomic promoter

In some embodiments, the nucleotide sequence encoding interleukin-12 comprises, from 5′ to 3′, a nucleotide sequence encoding p40, a nucleotide sequence encoding a linker, and nucleotide sequence encoding p35, and optionally wherein the nucleotide sequence encoding interleukin-12 is linked to a promoter located 5′ relative to the nucleotide sequence encoding p40.

In some embodiments, the srRNA comprises a 5′ cap untranslated region (UTR), one or more non-structural genes, a promoter, and a 3′ terminal polyadenylated (polyA) region.

In some embodiments, one or more non-structural genes comprises four non-structural genes (nsp1-4) and the promoter comprises a 26S subgenomic promoter.

In some embodiments, the nucleotide sequence encoding interleukin-12 is operably linked to the promoter.

In some embodiments, the nucleotide sequence encoding p40 is operably linked to the promoter.

In some embodiments, the srRNA comprises, from 5′ to 3′, the 5′ UTR, the one or more non-structural genes, the promoter, the nucleotide sequence encoding interleukin-12, and the 3′ polyA region.

In some embodiments, the srRNA lacks one or more nucleotide sequences encoding one or more structural protein sequences, optionally wherein the nucleotide sequence encoding interleukin-12 is inserted in place of the one or more nucleotide sequences encoding the one or more structural protein sequences.

In some embodiments, the srRNA is a TC-83 VEEV srRNA.

In some embodiments, the srRNA sequence comprises the sequence shown in SEQ ID NO: 5. In some embodiments, the srRNA sequence comprises a nucleic acid sequence having at least 99% sequence identity to the nucleic acid sequence of SEQ ID NO: 5. In some embodiments, the srRNA sequence comprising IL-12 consists of the sequence shown in SEQ ID NO: 10 OR 11. In some embodiments, the SrRNA sequence comprising IL-12 comprises the nucleic acid sequence of SEQ ID NO: 10 OR 11. In some embodiments, the SrRNA sequence comprising IL-12 comprises a nucleic acid sequence having at least 70% (e.g., 75%, 80%, 90%, 95%, 97%, 98%, or 99%) sequence identity to the nucleic acid sequence of SEQ ID NO: 10 OR 11.

In some embodiments, the srRNA comprises an mRNA cap.

In some embodiments, the mRNA cap comprises m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-, where Nm denotes any nucleotide with a 2′ O methylation.

In some embodiments, the srRNA is lyophilized.

In some embodiments, the lyophilized srRNA is at a temperature at or below 22° C., optionally about 2-8° C.

In some embodiments, there is a composition comprising the srRNA described herein and a delivery vehicle.

In some embodiments, the delivery vehicle comprises a lipid nanoparticle (LNP).

In some embodiments, the LNP comprises an ionizable cationic lipid.

In some embodiments, the LNP comprises an ionizable cationic lipid, heptadecan-9-yl 8-(3-(((4-(dimethylamino)butanoyl)oxy)methyl)-4-((8-(nonyloxy)-8-oxooctyl)oxy)phenoxy)octanoate. In some embodiments, the LNP comprises an ionizable lipid having the Formula:

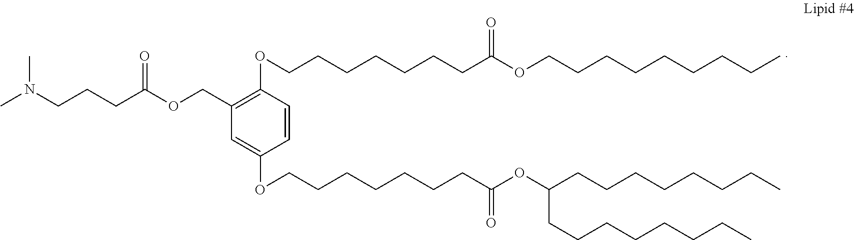 [SMILES: CCCCCCCCCOC(=O)CCCCCCCOc1ccc(OCCCCCCCC(=O)OC(CCCCCCCC)CCCCCCCC)cc1COC(=O)CCCN(C)C]

In some embodiments, the LNP comprises an ionizable lipid, 1,2-Diastearoyl-sn-glycero-3-phosphocholine (DSPC), Cholesterol, and 1,2-dimyristoyl-sn-glycero-3-phosphoethanolamine-N-[methoxy(polyethylene glycol)-2000] (DMG-PEG2000).

In some embodiments, the LNP comprises an ionizable lipid, 1,2-Dioleoyl-sn-glycero-3-phosphoethanolamine (DOPE), Cholesterol, and 1,2-dimyristoyl-sn-glycero-3-phosphoethanolamine-N-[methoxy(polyethylene glycol)-2000] (DMG-PEG2000).

In some embodiments, LNP comprises an ionizable lipid, 1,2-Dioleoyl-sn-glycero-3-phosphoethanolamine (DOPE), Cholesterol, and a pegylated lipid comprising a polyethylene glycol moiety.

In some embodiments, the srRNA is adsorbed to the surface of the LNP.

In some embodiments, the composition comprises a DSPC:cholesterol:PEG:ionizable lipid mole ratio ranging from 5:20:0:20 to 25:70:5:60, optionally 10:48:2:40, at a N:P (lipid:srRNA) ratio ranging from 2:1 to 12:1, optionally 8:1.

In some embodiments, the composition comprises a DOPE:cholesterol:PEG:ionizable lipid mole ratio ranging from 5:20:0:20 to 25:70:5:60, optionally 10:48:2:40, at a N:P (lipid:srRNA) ratio ranging from 2:1 to 12:1, optionally 8:1.

In some embodiments, the composition has a particle size of about 40-300 nm.

In some embodiments, the composition enhances an immune response in a subject following administration.

In some embodiments, the immune response comprises an antitumor immune response.

In some embodiments, the adaptive immune response comprises T cells and/or CD8+ T cells.

In some embodiments, the adaptive immune response comprises CD8+ T cells.

In some embodiments, the composition lacks a separate adjuvant component.

In some embodiments, there is a method of enhancing an immune response, comprising administering the composition of any embodiment to the subject.

In some embodiments, there is a method of treating a tumor in a subject, comprising administering the composition of an embodiment to the subject.

In some embodiments, the composition is administered to the subject intratumorally or intramuscularly.

In some embodiments, the composition is administered to the subject at least three times.

In some embodiments, the composition is administered once every one, two, three or four weeks.

In some embodiments, the composition is administered to the subject at a dose of 5-200 μg.

In some embodiments, the composition enhances an immune response in the subject following administration.

In some embodiments, there is a method using any of the above methods, further comprising the administration of a checkpoint inhibitor, optionally wherein the checkpoint inhibitor is administered prior to, concurrent with, or following administration of the srRNA or the composition.

In some embodiments, checkpoint inhibitor is a PD-1 inhibitor, PD-L1 inhibitor, or a combination thereof.

In some embodiments, the checkpoint inhibitor is an anti-PD-1 antibody, an anti-PD-L1 antibody.

In some embodiments, anti-PD1 or anti-PDL1 is administered to the subject at a dose of 3-10 mg/kg.

In some embodiments, the srRNA or the composition enhances an immune response in the subject following administration.

In some embodiments, the immune response comprises an antitumor immune response.

In some embodiments, the adaptive immune response comprises T cells and/or CD8+ cells.

In some embodiments, there is a method of making the composition of any embodiment, comprising mixing the srRNA with the delivery vehicle.

In some embodiments, the srRNA is lyophilized, and optionally at a temperature of 2-8° C.

In some embodiments, the delivery vehicle is an LNP, and optionally in a liquid state, optionally at a temperature of 2-8° C.

In some embodiments, there is a kit comprising the srRNA of any embodiment, a delivery vehicle, and instructions for use.

In some embodiments, the srRNA of the kit is lyophilized, and optionally at a temperature of 2-8° C.

In some embodiments, the delivery vehicle of the kit is an LNP, and optionally in a liquid state, optionally at a temperature of 2-8° C.

In some embodiments, the srRNA comprises a nucleic acid sequence having at least 80% sequence identity to the nucleic acid sequence of SEQ ID NO: 10 or 11.

In some embodiments, the srRNA comprises a nucleic acid sequence having at least 90% sequence identity to the nucleic acid sequence of SEQ ID NO: 10 or 11.

In some embodiments, the srRNA comprises a nucleic acid sequence having the nucleic acid sequence of SEQ ID NO: 10 or 11.

Described herein, in some embodiments, are compositions comprising: a) a self-replicating RNA (srRNA) comprising at least one nucleotide sequence encoding interleukin-12 (IL-12) comprising p40 and p35; and b) a lipid nanoparticle (LNP) comprising an ionizable lipid having the Formula:

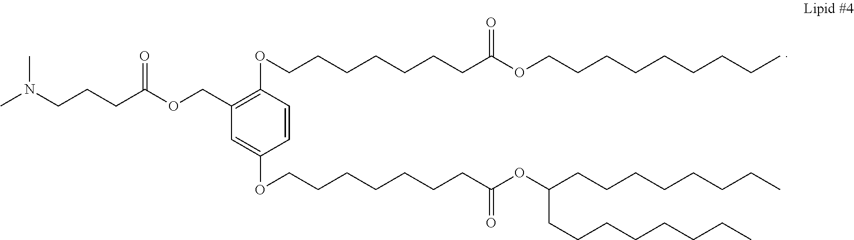 [SMILES: CCCCCCCCCOC(=O)CCCCCCCOc1ccc(OCCCCCCCC(=O)OC(CCCCCCCC)CCCCCCCC)cc1COC(=O)CCCN(C)C]

In some embodiments, the srRNA comprises a nucleic acid sequence having at least 80% sequence identity to the nucleic acid sequence of SEQ ID NO: 10 or 11. In some embodiments, the srRNA comprises a nucleic acid sequence having at least 90% sequence identity to the nucleic acid sequence of SEQ ID NO: 10 or 11. In some embodiments, the srRNA comprises a nucleic acid sequence having the nucleic acid sequence of SEQ ID NO: 10 or 11. In some embodiments, p40 and p35 are operably linked. In some embodiments, p40 and p35 are directly linked. In some embodiments, p40 and p35 are linked via a cleavable linker. In some embodiments, the interleukin-12 comprises human interleukin-12. In some embodiments, the interleukin-12 comprises human p40 and human p35. In some embodiments, p40 and p35 are operably linked. In some embodiments, p40 and p35 are directly linked. In some embodiments, p40 and p35 are linked via a cleavable linker. In some embodiments, interleukin-12 comprises one or more sequences comprising the sequences shown in SEQ ID NOs: 1. In some embodiments, interleukin-12 comprises one or more sequences consisting of the sequences shown in SEQ ID NOs: 1. In some embodiments, the sequence of p35 comprises the sequence shown in SEQ ID NO: 3. In some embodiments, the sequence of p35 consists of the sequence shown in SEQ ID NO: 3. In some embodiments, the sequence of p40 comprises the sequence shown in SEQ ID NO: 2. In some embodiments, the sequence of p40 consists of the sequence shown in SEQ ID NO: 2. In some embodiments, the sequence of the linker comprises the sequence shown in SEQ ID NO: 4. In some embodiments, in the sequence of the linker consists of the sequence shown in SEQ ID NO: 4. In some embodiments, the nucleotide sequence encoding interleukin-12 is operably linked to a promoter, optionally a subgenomic promoter. In some embodiments, the nucleotide sequence encoding interleukin-12 comprises, from 5′ to 3′, a nucleotide sequence encoding p40, a nucleotide sequence encoding a linker, and nucleotide sequence encoding p35, and optionally wherein the nucleotide sequence encoding interleukin-12 is linked to a promoter located 5′ relative to the nucleotide sequence encoding p40. In some embodiments, the srRNA comprises a 5′ cap untranslated region (UTR), one or more non-structural genes, a promoter, and a 3′ terminal polyadenylated (polyA) region. In some embodiments, the one or more non-structural genes comprises four non-structural genes (nsp1-4) and the promoter comprises a 26S subgenomic promoter. In some embodiments, the nucleotide sequence encoding interleukin-12 is operably linked to the promoter. In some embodiments, the nucleotide sequence encoding p40 is operably linked to the promoter. In some embodiments, the srRNA comprises, from 5′ to 3′, the 5′ UTR, the one or more non-structural genes, the promoter, the nucleotide sequence encoding interleukin-12, and the 3′ polyA region. In some embodiments, the srRNA lacks one or more nucleotide sequences encoding one or more structural protein sequences, optionally wherein the nucleotide sequence encoding interleukin-12 is inserted in place of the one or more nucleotide sequences encoding the one or more structural protein sequences. In some embodiments, the srRNA is a TC-83 VEEV srRNA. In some embodiments, the srRNA sequence comprises the sequence shown in SEQ ID NO: 5. In some embodiments, the srRNA sequence consists of the sequence shown in SEQ ID NO: 5. In some embodiments, the srRNA comprises an mRNA cap. In some embodiments, the mRNA cap comprises m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-, where Nm denotes any nucleotide with a 2′ O methylation. In some embodiments, the lyophilized srRNA is at a temperature at or below 22° C., optionally about 2-8° C. In some embodiments, the LNP comprises a cationic ionizable cationic lipid, 1,2-Diastearoyl-sn-glycero-3-phosphocholine (DSPC), Cholesterol, and 1,2-dimyristoyl-sn-glycero-3-phosphoethanolamine-N-[methoxy(polyethylene glycol)-2000] (DMG-PEG2000). In some embodiments, the LNP comprises a ionizable cationic lipid, 1,2-Dioleoyl-sn-glycero-3-phosphoethanolamine (DOPE), Cholesterol, and 1,2-dimyristoyl-sn-glycero-3-phosphoethanolamine-N-[methoxy(polyethylene glycol)-2000] (DMG-PEG2000). In some embodiments, the LNP comprises an ionizable cationic lipid, 1,2-Dioleoyl-sn-glycero-3-phosphoethanolamine (DOPE), Cholesterol, and a pegylated lipid comprising a polyethylene glycol moiety. In some embodiments, the srRNA is adsorbed to the surface of the LNP. In some embodiments, the composition comprises a DSPC:cholesterol:PEG:ionizable lipid mole ratio ranging from 5:20:0.5:20 to 25:70:5:60, optionally 10:48:2:40, at a N:P (lipid:srRNA) ratio ranging from 2:1 to 12:1, optionally 8:1. In some embodiments, the composition comprises a DOPE:cholesterol:PEG:ionizable lipid mole ratio ranging from 5:20:0:20 to 25:70:5:60, optionally 10:48:2:40, at a N:P (lipid:srRNA) ratio ranging from 2:1 to 12:1, optionally 8:1. In some embodiments, the composition has a particle size of about 40 to about 300 nanometer (nm). In some embodiments, the composition enhances an immune response in a subject following administration. In some embodiments, the immune response comprises an antitumor immune response. In some embodiments, the immune response comprises T cells and/or CD8+ cells. In some embodiments, the composition lacks a separate adjuvant component.

### BRIEF DESCRIPTION OF THE DRAWINGS

FIG. 1 shows the mean tumor volume of the C57BL/6 mice on the indicated days after start of treatment with liposome-packaged RNA replicon encoding IL-12 at the indicated dose levels.

FIG. 2 shows the percent survival of the C57BL/6 mice on the indicated days after start of treatment with liposome-packaged RNA replicon encoding IL-12 at the indicated dose levels.

FIG. 3 shows the average tumor volume of the right side, originally treated side, and left side, re-challenged side, C57BL/6 mice with an eliminated tumor after being treated with the indicated dose that were re-challenged with MC38 cells.

FIG. 4 shows ELISA assay results measuring the IL-12 concentration in tumor and serum samples of the mice on day 10 after start of treatment at the indicated doses.

FIG. 5 shows the quantity of the different immune cells analyzed by flow cytometry of the MC38 tumors collected on day 10 after starting treatment with liposome-packaged RNA replicon encoding IL-12.

FIG. 6 shows the mean tumor volume of the C57BL/6 mice on the indicated days after start of treatment with liposome-packaged RNA replicon encoding IL-12 and in combination with anti-mPD1 treatment at the indicated dose levels for the right side, or directly treated, tumor.

FIG. 7A shows the relative tumor volume (compared to Day 0) of the C57BL/6 mice on the indicated days after start of treatment with liposome-packaged RNA replicon encoding IL-12 and in combination with anti-mPD1 treatment at the indicated dose levels for the right side, or directly treated, tumor for the left side, or untreated, tumor. FIG. 7B shows the mean tumor volume of the C57BL/6 mice on the indicated days after start of treatment with liposome-packaged RNA replicon encoding IL-12 and in combination with anti-mPD1 treatment at the indicated dose levels for the right side, or directly treated, tumor for the left side, or untreated, tumor.

FIG. 8 shows the quantity of the different immune cells analyzed by flow cytometry of the MC38 tumors collected on day 10 after starting treatment with liposome-packaged RNA replicon encoding IL-12 and in combination with anti-mPDI.

FIG. 9 shows an exemplary srRNA IL-12 construct.

FIG. 10 shows the mean B16 tumor volume of the C57BL/6 mice on the indicated days after start of weekly intravenous treatment with liposome-packaged RNA replicon encoding IL-12 at the indicated dose levels.

FIG. 11 shows the probability of survival of the C57BL/6 mice with B16 tumor-bearing on the indicated days after start of weekly intravenous treatment with liposome-packaged RNA replicon encoding IL-12 at the indicated dose levels.

FIG. 12 showed the changes of IL-12 and IFN-γ in tumor and serum samples of the mice on day 10 after start of weekly intravenous treatment with liposome-packaged RNA replicon encoding IL-12 at the indicated doses.

FIGS. 13A-13E shows the percentage of various immune cells analyzed by flow cytometry of tumor samples collected on day 10 after starting weekly intravenous treatment with liposome-packaged RNA replicon encoding IL-12 at the indicated doses.

FIG. 14 shows the expression of cytokines analyzed by flow cytometry of the tumor samples collected on day 10 after starting weekly intravenous treatment with liposome-packaged RNA replicon encoding IL-12 at the indicated doses.

FIG. 15 shows the NSP4 expression levels in blood cells and tumors on day 10 after starting weekly intravenous treatment with liposome-packaged RNA replicon encoding IL-12 at the indicated doses.

FIG. 16 shows the mean B16 tumor volume of the C57BL/6 mice on the indicated days after starting biweekly intravenous treatment with liposome-packaged RNA replicon encoding IL-12 at the indicated doses.

FIG. 17 shows the probability of survival of the C57BL/6 mice with B16 tumor on the indicated days after tumor inoculation followed by biweekly intravenous treatment with liposome-packaged RNA replicon encoding IL-12 at the indicated doses.

FIG. 18 shows the mean EMT6 tumor volume of the Balb/c mice on the indicated days after starting biweekly intravenous treatment with liposome-packaged RNA replicon encoding IL-12 at the indicated doses.

FIG. 19 shows the probability of survival of the Balb/c mice with EMT6 tumor on the indicated days after tumor inoculation followed by biweekly intravenous treatment with liposome-packaged RNA replicon encoding IL-12 at the indicated doses.

### DETAILED DESCRIPTION

### IL-12

Interleukin-12 (IL-12) is a proinflammatory cytokine that induces the production of interferon-gamma (IFN-7), promotes the differentiation of T helper-1 (Th1) cells and connects innate and adaptive immune response pathways (Trinchieri, Nat Rev Immunol (2003) 3:133). IL-12 is produced by dendritic cells (DC) and phagocytes (e.g., macrophages, neutrophils, immature dendritic cells) in response to pathogens during infection. Structurally, IL-12 is a heterodimeric protein comprised of two polypeptide chains, a p35 chain and a p40 chain (Airoldi, et al., Haematologica (2002) 87:434-42). IL-12 is structurally related to at least two other heterodimeric proinflammatory cytokines, interleukin-23 (IL-23) and interleukin-27 (IL-27) (Hunter, Nat Rev Immunol (2005) 5:521; and Vandenbroeck, et al., J Pharm Pharmacol (2004) 56:145).

### Polynucleotides

The liposome packaged RNA replicons encoding IL-12, as provided herein, comprise at least one self-replicating ribonucleic acid (srRNA) polynucleotide encoding IL-12. The term “nucleic acid,” in its broadest sense, includes any compound and/or substance that comprises a polymer of nucleotides. These polymers are referred to as polynucleotides.

In some embodiments, provided herein is a self-replicating messenger RNA (srRNA) comprising at least one nucleotide sequence encoding interleukin-12 (IL-12) comprising p40 and p35.

In some embodiments, p40 and p35 are operably linked.

In some embodiments, p40 and p35 are directly linked.

In some embodiments, p40 and p35 are linked via a cleavable linker.

In some embodiments, the interleukin-12 comprises human interleukin-12.

In some embodiments, the interleukin-12 comprises human p40 and human p35.

In some embodiments, the interleukin-12 comprises a nucleic acid sequence of SEQ ID NO: 1. In some embodiments, the interleukin-12 comprises a nucleic acid sequence having at least 70% (e.g., 75%, 80%, 90%, 95%, 97%, 98%, or 99%) sequence identity to the nucleic acid sequence of SEQ ID NO: 1. In some embodiments, the interleukin-12 comprises a nucleic acid sequence having at least 75% sequence identity to the nucleic acid sequence of SEQ ID NO: 1. In some embodiments, the interleukin-12 comprises a nucleic acid sequence having at least 80% sequence identity to the nucleic acid sequence of SEQ ID NO: 1. In some embodiments, the interleukin-12 comprises a nucleic acid sequence having at least 90% sequence identity to the nucleic acid sequence of SEQ ID NO: 1. In some embodiments, the interleukin-12 comprises a nucleic acid sequence having at least 95% sequence identity to the nucleic acid sequence of SEQ ID NO: 1. In some embodiments, the interleukin-12 comprises a nucleic acid sequence having at least 97% sequence identity to the nucleic acid sequence of SEQ ID NO: 1. In some embodiments, the interleukin-12 comprises a nucleic acid sequence having at least 98% sequence identity to the nucleic acid sequence of SEQ ID NO: 1. In some embodiments, the interleukin-12 comprises a nucleic acid sequence having at least 99% sequence identity to the nucleic acid sequence of SEQ ID NO: 1.

In some embodiments, the p35 comprises the sequence shown in SEQ ID NO: 3. In some embodiments, the p35 consists of the sequence shown in SEQ ID NO: 3. In some embodiments, at least one RNA polynucleotide of IL-12 is encoded by SEQ ID NO: 3. In some embodiments, the p35 sequence comprises the nucleic acid sequence of SEQ ID NO: 3. In some embodiments, the p35 sequence comprises a nucleic acid sequence having at least 70% (e.g., 75%, 80%, 90%, 95%, 97%, 98%, or 99%) sequence identity to the nucleic acid sequence of SEQ ID NO: 3. In some embodiments, the p35 sequence comprises a nucleic acid sequence having at least 75% sequence identity to the nucleic acid sequence of SEQ ID NO: 3. In some embodiments, the p35 sequence comprises a nucleic acid sequence having at least 80% sequence identity to the nucleic acid sequence of SEQ ID NO: 3. In some embodiments, the p35 sequence comprises a nucleic acid sequence having at least 90% sequence identity to the nucleic acid sequence of SEQ ID NO: 3. In some embodiments, the p35 sequence comprises a nucleic acid sequence having at least 95% sequence identity to the nucleic acid sequence of SEQ ID NO: 3. In some embodiments, the p35 sequence comprises a nucleic acid sequence having at least 97% sequence identity to the nucleic acid sequence of SEQ ID NO: 3. In some embodiments, the p35 sequence comprises a nucleic acid sequence having at least 98% sequence identity to the nucleic acid sequence of SEQ ID NO: 3. In some embodiments, the p35 sequence comprises a nucleic acid sequence having at least 99% sequence identity to the nucleic acid sequence of SEQ ID NO: 3.

In some embodiments, the sequence of p40 comprises the sequence shown in SEQ ID NO: 2.

In some embodiments, the sequence of p40 consists of the sequence shown in SEQ ID NO: 2. In some embodiments, at least one RNA polynucleotide of IL-12 is encoded by SEQ ID NO: 2. In some embodiments, the p40 sequence comprises the nucleic acid sequence of SEQ ID NO: 2. In some embodiments, the p40 sequence comprises a nucleic acid sequence having at least 70% (e.g., 75%, 80%, 90%, 95%, 97%, 98%, or 99%) sequence identity to the nucleic acid sequence of SEQ ID NO: 2. In some embodiments, the p40 sequence comprises a nucleic acid sequence having at least 75% sequence identity to the nucleic acid sequence of SEQ ID NO: 2. In some embodiments, the p40 sequence comprises a nucleic acid sequence having at least 80% sequence identity to the nucleic acid sequence of SEQ ID NO: 2. In some embodiments, the p40 sequence comprises a nucleic acid sequence having at least 90% sequence identity to the nucleic acid sequence of SEQ ID NO: 2. In some embodiments, the p40 sequence comprises a nucleic acid sequence having at least 95% sequence identity to the nucleic acid sequence of SEQ ID NO: 2. In some embodiments, the p40 sequence comprises a nucleic acid sequence having at least 97% sequence identity to the nucleic acid sequence of SEQ ID NO: 2. In some embodiments, the p40 sequence comprises a nucleic acid sequence having at least 98% sequence identity to the nucleic acid sequence of SEQ ID NO: 2. In some embodiments, the p40 sequence comprises a nucleic acid sequence having at least 99% sequence identity to the nucleic acid sequence of SEQ ID NO: 2.

In some embodiments, the sequence of the linker comprises the sequence shown in SEQ ID NO: 4. In some embodiments, the sequence of the linker consists of the sequence shown in SEQ ID NO: 4. In some embodiments, the sequence of the linker comprises 5, 10, 15, 20, 25, 30, 35, 40, or more than 40 nucleotides of SEQ ID NO: 4. In some embodiments, the sequence of the linker comprises 5, 10, 15, 20, 25, 30, 35, 40, or more than 40 consecutive nucleotides of SEQ ID NO: 4

In some embodiments, the nucleotide sequence encoding interleukin-12 is operably linked to a promoter, optionally a subgenomic promoter. In some embodiments, the nucleotide sequence encoding p40 is operably linked to the promoter.

In some embodiments, the nucleotide sequence encoding interleukin-12 comprises, from 5′ to 3′, a nucleotide sequence encoding p40, a nucleotide sequence encoding a linker, and nucleotide sequence encoding p35, and optionally wherein the nucleotide sequence encoding interleukin-12 is linked to a promoter located 5′ relative to the nucleotide sequence encoding p40.

Described herein, in some embodiments, are srRNA molecules or vectors comprising IL-12. In some embodiments, the srRNA comprises a 5′ cap untranslated region (UTR), one or more non-structural genes, a promoter, and a 3′ terminal polyadenylated (polyA) region.

In some embodiments, one or more non-structural genes comprises four non-structural genes (nsp1-4) and the promoter comprises a 26S subgenomic promoter.

In some embodiments, the nucleotide sequence encoding interleukin-12 is operably linked to the promoter.

In some embodiments, the srRNA comprises, from 5′ to 3′, the 5′ UTR, the one or more non-structural genes, the promoter, the nucleotide sequence encoding interleukin-12, and the 3′ polyA region.

In some embodiments, the srRNA lacks one or more nucleotide sequences encoding one or more structural protein sequences, optionally wherein the nucleotide sequence encoding interleukin-12 is inserted in place of the one or more nucleotide sequences encoding the one or more structural protein sequences.

In some embodiments, the srRNA is a TC-83 VEEV srRNA

In some embodiments, the srRNA sequence comprises the sequence shown in SEQ ID NO: 5.

In some embodiments, the srRNA sequence comprises the nucleic acid sequence of SEQ ID NO: 5. In some embodiments, the srRNA sequence comprises a nucleic acid sequence having at least 70% (e.g., 75%, 80%, 90%, 95%, 97%, 98%, or 99%) sequence identity to the nucleic acid sequence of SEQ ID NO: 5. In some embodiments, the srRNA sequence comprises a nucleic acid sequence having at least 75% sequence identity to the nucleic acid sequence of SEQ ID NO: 5. In some embodiments, the srRNA sequence comprises a nucleic acid sequence having at least 80% sequence identity to the nucleic acid sequence of SEQ ID NO: 5. In some embodiments, the srRNA sequence comprises a nucleic acid sequence having at least 90% sequence identity to the nucleic acid sequence of SEQ ID NO: 5. In some embodiments, the srRNA sequence comprises a nucleic acid sequence having at least 95% sequence identity to the nucleic acid sequence of SEQ ID NO: 5. In some embodiments, the srRNA sequence comprises a nucleic acid sequence having at least 97% sequence identity to the nucleic acid sequence of SEQ ID NO: 5. In some embodiments, the srRNA sequence comprises a nucleic acid sequence having at least 98% sequence identity to the nucleic acid sequence of SEQ ID NO: 5. In some embodiments, the srRNA sequence comprises a nucleic acid sequence having at least 99% sequence identity to the nucleic acid sequence of SEQ ID NO: 5.

In some embodiments, the srRNA sequence comprising IL-12 consists of the sequence shown in SEQ ID NO: 10 OR 11. In some embodiments, the SrRNA sequence comprising IL-12 comprises the nucleic acid sequence of SEQ ID NO: 10 OR 11. In some embodiments, the SrRNA sequence comprising IL-12 comprises a nucleic acid sequence having at least 70% (e.g., 75%, 80%, 90%, 95%, 97%, 98%, or 99%) sequence identity to the nucleic acid sequence of SEQ ID NO: 10 OR 11. In some embodiments, the SrRNA sequence comprising IL-12 comprises a nucleic acid sequence having at least 75% sequence identity to the nucleic acid sequence of SEQ ID NO: 10 OR 11. In some embodiments, the SrRNA sequence comprising IL-12 comprises a nucleic acid sequence having at least 80% sequence identity to the nucleic acid sequence of SEQ ID NO: 10 OR 11. In some embodiments, the SrRNA sequence comprising IL-12 comprises a nucleic acid sequence having at least 90% sequence identity to the nucleic acid sequence of SEQ ID NO: 10 OR 11. In some embodiments, the SrRNA sequence comprising IL-12 comprises a nucleic acid sequence having at least 95% sequence identity to the nucleic acid sequence of SEQ ID NO: 10 OR 11. In some embodiments, the SrRNA sequence comprising IL-12 comprises a nucleic acid sequence having at least 97% sequence identity to the nucleic acid sequence of SEQ ID NO: 10 OR 11. In some embodiments, the SrRNA sequence comprising IL-12 comprises a nucleic acid sequence having at least 98% sequence identity to the nucleic acid sequence of SEQ ID NO: 10 OR 11. In some embodiments, the SrRNA sequence comprising IL-12 comprises a nucleic acid sequence having at least 99% sequence identity to the nucleic acid sequence of SEQ ID NO: 10 OR 11.

In some embodiments, polynucleotides of the present disclosure function as self-replicating RNA (srRNA). “srRNA” refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence, but where the sequence represents RNA (e.g., srRNA), the “T”s would be substituted for “U”s.

Thus, any of the RNA polynucleotides encoded by a DNA identified by a particular sequence identification number may also comprise the corresponding RNA (e.g., srRNA) sequence encoded by the DNA, where each “T” of the DNA sequence is substituted with “U.”

It should be understood that the RNA polynucleotides of the liposome packaged RNA replicons encoding IL-12 as provided herein are synthetic molecules, i.e., they are not naturally-occurring molecules. That is, the RNA polynucleotides of the present disclosure are isolated RNA polynucleotides. As is known in the art, “isolated polynucleotides” refer to polynucleotides that are substantially physically separated from other cellular material (e.g., separated from cells and/or systems that produce the polynucleotides) or from other material that hinders their use in the liposome packaged RNA replicon encoding IL-12 of the present disclosure. Isolated polynucleotides are substantially pure in that they have been substantially separated from the substances with which they may be associated in living or viral systems. Thus, liposome packaged RNA replicons are not associated with living or viral systems, such as cells or viruses. The liposome packaged RNA replicons do not include viral components (e.g., viral capsids, viral enzymes, or other viral proteins, for example, those needed for viral-based replication), and the liposome packaged RNA replicons are not packaged within, encapsulated within, linked to, or otherwise associated with a virus or viral particle. In some embodiments, the liposome packaged RNA replicons comprise a lipid nanoparticle that consists of, or consists essentially of, one RNA polynucleotide (e.g., RNA polynucleotides encoding IL-12)

In some embodiments, a sequence encoding IL-12 is codon optimized. Any one or more of the sequences provided herein may be codon optimized. Codon optimization methods are known in the art. Codon optimization, in some embodiments, may be used to match codon frequencies in target and host organisms to ensure proper folding; bias GC content to increase RNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA degradation sites; adjust translational rates to allow the various domains of the protein to fold properly; or reduce or eliminate problem secondary structures within the polynucleotide. In some embodiments, the open reading frame (ORF) sequence is optimized using optimization algorithms.

In some embodiments, the liposome packaged RNA replicon encoding IL-12 includes at least one RNA polynucleotide encoding at least one IL-12 polypeptide having at least one of: a modification, at least one 5′ terminal cap, and formulation with a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

When transfected into mammalian cells, the modified RNAs typically have a stability of between 12-18 hours, or greater than 18 hours, e.g., 24, 36, 48, 60, 72, or greater than 72 hours.

### In Vitro Transcription of RNA

IL-12 srRNA delivery particles of the present disclosure comprise at least one RNA polynucleotide, such as a srRNA. RNA, for example, is transcribed in vitro from template DNA, referred to as an “in vitro transcription template.”

In vitro transcription of RNA is known in the art and is described, e.g., in International Publication WO2014/152027, which is incorporated by reference herein in its entirety. For example, in some embodiments, the RNA transcript is generated using a non-amplified, linearized DNA template in an in vitro transcription reaction to generate the RNA transcript. In some embodiments the RNA transcript is capped via enzymatic capping. In some embodiments the RNA transcript is purified via chromatographic methods, e.g., use of an oligo dT substrate. Some embodiments exclude the use of DNase. In some embodiments the RNA transcript is synthesized from a non-amplified, linear DNA template coding for the gene of interest via an enzymatic in vitro transcription reaction utilizing a T7 phage RNA polymerase and nucleotide triphosphates of the desired chemistry. Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides.

As used herein, the terms “termini” or “terminus,” when referring to polypeptides or polynucleotides, refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N-termini and C-termini. Alternatively, the termini of the polypeptides may be modified such that they begin or end, as the case may be, with a non-polypeptide based moiety such as an organic conjugate.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the RNA encoded by the template.

A “5′ untranslated region” (UTR) refers to a region of a RNA that is directly upstream (i.e., 5′) from the start codon (i.e., the first codon of an RNA transcript translated by a ribosome) that does not encode a polypeptide.

A “3′ untranslated region” (UTR) refers to a region of an RNA that is directly downstream (i.e., 3′) from the stop codon (i.e., the codon of an RNA transcript that signals a termination of translation) that does not encode a polypeptide.

In some embodiments, the RNA described herein has an elongated 3′ UTR. In some embodiments, the RNA described herein has a 3′ UTR between 100-500 nucleotides in length. In some embodiments, the RNA described herein has a 3′ UTR between 125-500 nucleotides in length. In some embodiments, the RNA described herein has a 3′ UTR between 150-500 nucleotides in length. In some embodiments, the RNA described herein has a 3′ UTR between 175-500 nucleotides in length. In some embodiments, the RNA described herein has a 3′ UTR between 200-500 nucleotides in length. In some embodiments, the RNA described herein has a 3′ UTR between 225-500 nucleotides in length. In some embodiments, the RNA described herein has a 3′ UTR between 250-500 nucleotides in length. In some embodiments, the RNA described herein has a 3′ UTR between 275-500 nucleotides in length. In some embodiments, the RNA described herein has a 3′ UTR between 300-500 nucleotides in length. In some embodiments, the RNA described herein has a 3′ UTR between 325-500 nucleotides in length. In some embodiments, the RNA described herein has a 3′ UTR between 100-476 nucleotides in length. In some embodiments, the RNA described herein has a 3′ UTR between 100-450 nucleotides in length. In some embodiments, the RNA described herein has a 3′ UTR between 100-425 nucleotides in length. In some embodiments, the RNA described herein has a 3′ UTR between 100-400 nucleotides in length. In some embodiments, the RNA described herein has a 3′ UTR between 100-375 nucleotides in length. In some embodiments, the RNA described herein has a 3′ UTR between 100-350 nucleotides in length. In some embodiments, the RNA described herein has a 3′ UTR between 300-350 nucleotides in length. In some embodiments, the RNA described herein has a 3′ UTR about 330 nucleotides in length. In some embodiments, the RNA described herein comprises a 3′ UTR of 50, 75, 100, 125, 150, 175, 200, 225, 250, 275, 300, 325, 350, 375, 400 or more than 440 nucleotides in length.

An “open reading frame” is a continuous stretch of DNA or RNA beginning with a start codon (e.g., methionine (ATG or AUG)), and ending with a stop codon (e.g., TAA, TAG or TGA, or UAA, UAG or UGA) and typically encodes a polypeptide (e.g., protein). It will be understood that the sequences disclosed herein may further comprise additional elements, e.g., 5′ and 3′ UTRs, but that those elements, unlike the ORF, need not necessarily be present in a liposome packaged RNA replicon of the present disclosure.

A “polyA tail” is a region of RNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect RNA from enzymatic degradation, e.g., in the cytoplasm, and aids in transcription termination, and/or export of the RNA from the nucleus and translation.

In some embodiments, the RNA described herein comprises an elongated polyA tail. In some embodiments, the RNA described herein comprises a polyA tail between 30-100 nucleotides in length. In some embodiments, the RNA described herein comprises a polyA tail between 35-100 nucleotides in length. In some embodiments, the RNA described herein comprises a polyA tail between 40-100 nucleotides in length. In some embodiments, the RNA described herein comprises a polyA tail between 45-100 nucleotides in length. In some embodiments, the RNA described herein comprises a polyA tail between 50-100 nucleotides in length. In some embodiments, the RNA described herein comprises a polyA tail between 55-100 nucleotides in length. In some embodiments, the RNA described herein comprises a polyA tail between 60-100 nucleotides in length. In some embodiments, the RNA described herein comprises a polyA tail between 65-100 nucleotides in length. In some embodiments, the RNA described herein comprises a polyA tail between 70-100 nucleotides in length. In some embodiments, the RNA described herein comprises a polyA tail between 80-100 nucleotides in length. In some embodiments, the RNA described herein comprises a polyA tail between 90-100 nucleotides in length. In some embodiments, the RNA described herein comprises a polyA tail between 95-100 nucleotides in length. In some embodiments, the RNA described herein comprises a polyA tail between 30-95 nucleotides in length. In some embodiments, the RNA described herein comprises a polyA tail between 30-90 nucleotides in length. In some embodiments, the RNA described herein comprises a polyA tail between 30-85 nucleotides in length. In some embodiments, the RNA described herein comprises a polyA tail between 30-80 nucleotides in length. In some embodiments, the RNA described herein comprises a polyA tail between 30-75 nucleotides in length. In some embodiments, the RNA described herein comprises a polyA tail between 30-70 nucleotides in length. In some embodiments, the RNA described herein comprises a polyA tail between 30-65 nucleotides in length. In some embodiments, the RNA described herein comprises a polyA tail between 30-60 nucleotides in length. In some embodiments, the RNA described herein comprises a polyA tail between 30-55 nucleotides in length. In some embodiments, the RNA described herein comprises a polyA tail between 30-50 nucleotides in length. In some embodiments, the RNA described herein comprises a polyA tail between 30-45 nucleotides in length. In some embodiments, the RNA described herein comprises a polyA tail of 10, 15, 20, 25, 30, 35, 40, 45, 50, 55, 60, 65, 70, 75, 80, 85, 90, 95, 100, 110, 120, 130, 140, or more than 140 nucleotides in length.

### Nanoparticle Formulations

Particles can be non-virion particles, i.e., they are not a virion. Thus, in some embodiments, the particle does not comprise a protein capsid. By avoiding the need to create a capsid particle, a particle does not utilize a packaging cell line, thus permitting easier up-scaling for commercial production and minimizing the risk that dangerous infectious viruses will inadvertently be produced. Particles described herein can be formed from a delivery material. Various materials are suitable for forming particles which can deliver RNA to a vertebrate cell in vivo. Two delivery materials are (i) amphiphilic lipids which can form liposomes and (ii) non-toxic and biodegradable polymers which can form microparticles. Other delivery methods may include, but are not limited to, exosomes and cationic nano-emulsion.

Where delivery is by liposome, RNA can be encapsulated; where delivery is by polymeric microparticle, RNA can be encapsulated or adsorbed. A third delivery material is the particulate reaction product of a polymer, a crosslinker, a RNA, and a charged monomer. In one embodiment, the particle described herein comprises a liposome encapsulating a self-replicating RNA molecule which encodes an immunogen. RNA can be encapsulated within the particles, particularly if the particle is a liposome. This means that RNA inside the particles is separated from any external medium by the delivery material, and encapsulation has been found to protect RNA from RNase digestion. Encapsulation can take various forms. For example, in some embodiments, the delivery material forms a outer layer around an aqueous RNA-containing core. RNA can be adsorbed to the particles. This means, in some embodiments, that RNA is not separated from any external medium by the delivery material, unlike the RNA genome of a natural virus.

In some embodiments, IL-12 RNA (e.g., srRNA) particles are formulated in a nanoparticle. In some embodiments, IL-12 RNA particles are formulated in a lipid nanoparticle.

In some embodiments, a IL-12 RNA (e.g., srRNA) particles formulation is a nanoparticle that comprises at least one lipid. The lipid may be a biodegradable, cationic lipid. Useful cationic lipids generally contain a nitrogen atom that is positively charged under physiological conditions e.g. as a tertiary or quaternary amine. This nitrogen can be in the hydrophilic head group of an amphiphilic surfactant. The lipid may be selected from, but is not limited to, 1,2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimetylammonium salts, [1-(2,3-dioleyloxy)-propyl]-N,N,N,trimethylammonium chloride, 1,2-diacyl-3-(trimethylammonio)propane (acyl group=dimyristoyl, dipalmitoyl, distearoyl, dioleoyl), 1,2-diacyl-3-(dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio)butanoate), N-alkyl pyridinium salts (e.g. cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C₁₂GluPhCₙN⁺), ditetradecyl glutamate ester with pendant amino group (C12GluPhCₙN^{|}), cationic derivatives of cholesterol, including but not limited to cholesteryl-3β-oxysuccinamidoethylenetrimethylammonium salt, cholesteryl-3β-oxysuccinamidoethylenedimethylamine, cholesteryl-3β-carboxyamidoethylenetrimethylammonium salt, and cholesteryl-3β-carboxyamidoethylenedimethylamine.

In some embodiments, a IL-12 RNA particle is a nanoparticle that comprises at least one lipid. The lipid may be a neutral lipid. The lipid may be a phospholipid. The phospholipid may be selected from, but is not limited to, DDPC, 1,2-Didecanoyl-sn-Glycero-3-phosphatidylcholine, DEPA, 1,2-Dierucoyl-sn-Glycero-3-Phosphate, DEPC, 1,2-Erucoyl-sn-Glycero-3-phosphatidylcholine, DEPE, 1,2-Dierucoyl-sn-Glycero-3-phosphatidylethanolamine, DEPG, 1,2-Dierucoyl-sn-Glycero-3[Phosphatidyl-rac-(1-glycerol . . . ), DLOPC 1,2-Linoleoyl-sn-Glycero-3-phosphatidylcholine, DLPA, 1,2-Dilauroyl-sn-Glycero-3-Phosphate, DLPC, 1,2-Dilauroyl-sn-Glycero-3-phosphatidylcholine, DLPE 1,2-Dilauroyl-sn-Glycero-3-phosphatidylethanolamine, DLPG 1,2-Dilauroyl-sn-Glycero-3[Phosphatidyl-rac-(1-glycerol . . . ), DLPS 1,2-Dilauroyl-sn-Glycero-3-phosphatidylserine, DMG, 1,2-Dimyristoyl-sn-glycero-3-phosphoethanolamine, DMPA, 1,2-Dimyristoyl-sn-Glycero-3-Phosphate, DMPC, 1,2-Dimyristoyl-sn-Glycero-3-phosphatidylcholine, DMPE, 1,2-Dimyristoyl-sn-Glycero-3-phosphatidylethanolamine, DMPG, 1,2-Myristoyl-sn-Glycero-3[Phosphatidyl-rac-(1-glycerol . . . ), DMPS, 1,2-Dimyristoyl-sn-Glycero-3-phosphatidylserine, DOPA, 1,2-Dioleoyl-sn-Glycero-3-Phosphate, DOPC, 1,2-Dioleoyl-sn-Glycero-3-phosphatidylcholine, DOPE, 1,2-Dioleoyl-sn-Glycero-3-phosphatidylethanolamine, DOPG, 1,2-Dioleoyl-sn-Glycero-3[Phosphatidyl-rac-(1-glycerol . . . ), DOPS, 1,2-Dioleoyl-sn-Glycero-3-phosphatidylserine, DPPA, 1,2-Dipalmitoyl-sn-Glycero-3-Phosphate, DPPC, 1,2-Dipalmitoyl-sn-Glycero-3-phosphatidylcholine, DPPE, 1,2-Dipalmitoyl-sn-Glycero-3-phosphatidylethanolamine, DPPG, 1,2-Dipalmitoyl-sn-Glycero-3[Phosphatidyl-rac-(1-glycerol . . . ), DPPS, 1,2-Dipalmitoyl-sn-Glycero-3-phosphatidylserine, DPyPE, 1,2-diphytanoyl-sn-glycero-3-phosphoethanolamine, DSPA, 1,2-Distearoyl-sn-Glycero-3-Phosphate, DSPC, 1,2-Distearoyl-sn-Glycero-3-phosphatidylcholine, DSPE, 1,2-Diostearpyl-sn-Glycero-3-phosphatidylethanolamine, DSPG, 1,2-Distearoyl-sn-Glycero-3[Phosphatidyl-rac-(1-glycerol . . . ), DSPS, 1,2-Distearoyl-sn-Glycero-3-phosphatidylserine, EPC, Egg-PC, HEPC, Hydrogenated Egg PC, HSPC, High purity Hydrogenated Soy PC, HSPC, Hydrogenated Soy PC, LYSOPC MYRISTIC, 1-Myristoyl-sn-Glycero-3-phosphatidylcholine, LYSOPC PALMITIC, 1-Palmitoyl-sn-Glycero-3-phosphatidylcholine, LYSOPC STEARIC, 1-Stearoyl-sn-Glycero-3-phosphatidylcholine, Milk Sphingomyelin, MPPC, 1-Myristoyl,2-palmitoyl-sn-Glycero 3-phosphatidyl choline, MSPC, 1-Myristoyl,2-stearoyl-sn-Glycero-3-phosphatidylcholine, PMPC, 1-Palmitoyl,2-myristoyl-sn-Glycero-3-phosphatidylcholine, POPC, 1-Palmitoyl,2-oleoyl-sn-Glycero-3-phosphatidylcholine, POPE, 1-Palmitoyl-2-oleoyl-sn-Glycero-3-phosphatidylethanolamine, POPG, 1,2-Dioleoyl-sn-Glycero-3[Phosphatidyl-rac-(1-glycerol) . . . ], PSPC, 1-Palmitoyl,2-stearoyl-sn-Glycero-3-phosphatidylcholine, SMPC, 1-Stearoyl,2-myristoyl-sn-Glycero-3-phosphatidylcholine, SOPC, 1-Stearoyl,2-oleoyl-sn-Glycero-3-phosphatidylcholine, SPPC, 1-Stearoyl,2-palmitoyl-sn-Glycero-3-phosphatidylcholine.

In some embodiments, the LNP comprises an ionizable cationic lipid.

In some embodiments, the LNP comprises an ionizable cationic lipid, heptadecan-9-yl 8-(3-(((4-(dimethylamino)butanoyl)oxy)methyl)-4-((8-(nonyloxy)-8-oxooctyl)oxy)phenoxy)octanoate. In some embodiments, the LNP comprises an ionizable lipid with the formula:

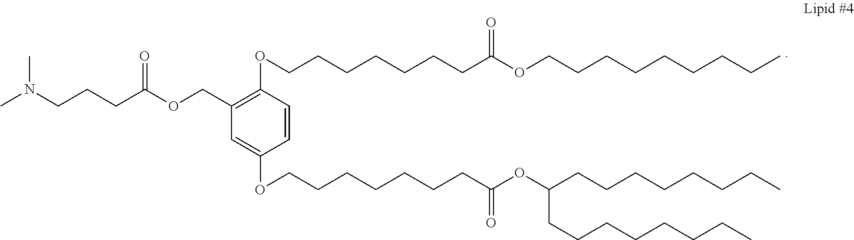 [SMILES: CCCCCCCCCOC(=O)CCCCCCCOc1ccc(OCCCCCCCC(=O)OC(CCCCCCCC)CCCCCCCC)cc1COC(=O)CCCN(C)C]

In some embodiments, the LNP comprises an ionizable lipid, 1,2-Diastearoyl-sn-glycero-3-phosphocholine (DSPC), Cholesterol, and 1,2-dimyristoyl-sn-glycero-3-phosphoethanolamine-N-[methoxy(polyethylene glycol)-2000] (DMG-PEG2000).

In some embodiments, the LNP comprises an ionizable lipid, 1,2-Dioleoyl-sn-glycero-3-phosphoethanolamine (DOPE), Cholesterol, and 1,2-dimyristoyl-sn-glycero-3-phosphoethanolamine-N-[methoxy(polyethylene glycol)-2000] (DMG-PEG2000).

In some embodiments, LNP comprises an ionizable lipid, 1,2-Dioleoyl-sn-glycero-3-phosphoethanolamine (DOPE), Cholesterol, and a pegylated lipid comprising a polyethylene glycol moiety.

In some embodiments, the srRNA is adsorbed to the surface of the LNP.

In some embodiments, the composition comprises a DSPC:cholesterol:PEG:ionizable lipid mole ratio ranging from 5:20:0:20 to 25:70:5:60, optionally 10:48:2:40, at a N:P (lipid:srRNA) ratio ranging from 2:1 to 12:1, optionally 8:1.

In some embodiments, the composition further comprises one or more buffers, salts, or sugars. In some embodiments, the composition further comprises, Tris, NaCl, sucrose, or combinations thereof. In some embodiments, the Tris is provided at a concentration in a range of about 5 to about 40, about 10 to about 30, or about 15 to about 25 mmol/L. In some embodiments, the Tris is provided at a concentration of about 20 mmol/L. In some embodiments, the NaCl is provided at a concentration in a range of about 1 to about 20, about 2 to about 15, or about 1 to about 10 mmol/L. In some embodiments, the NaCl is provided at a concentration of about 5 mmol/L. In some embodiments, the sucrose is provided in a range of about 1% to about 20%, about 2% to about 15%, or about 1% to about 10%. In some embodiments, the sucrose is provided at about 7.5%.

In some embodiments, the composition comprises a DOPE:cholesterol:PEG:ionizable lipid mole ratio ranging from 5:20:0:20 to 25:70:5:60, optionally 10:48:2:40, at a N:P (lipid:srRNA) ratio ranging from 2:1 to 12:1, optionally 8:1.

In some embodiments, the composition has a particle size of about 40-300 nm.

In some embodiments, a lipid nanoparticle formulation consists essentially of (i) a neutral phospholipid (ii) a sterol, e.g., cholesterol; (iii) a pegylated lipid comprising a polyethylene glycol moiety and (iv) a cationic lipid at a molar ratio ranging from 5:20:0:20 to 25:70:5:60, optionally 10:48:2:40, at a N:P (lipid:srRNA) ratio ranging from 2:1 to 12:1, optionally 8:1.

In some embodiments, the liposome packaged RNA replicons of the present disclosure may be formulated in lipid nanoparticles having a diameter 40-300 nm.

### RNA

The RNA described herein can be a self-replicating RNA (srRNA). A self-replicating RNA molecule (replicon) can, when delivered to a vertebrate cell, lead to the production of multiple daughter RNAs by transcription from itself (via an antisense copy which it generates from itself). A self-replicating RNA molecule can thus typically a +-strand molecule which can be directly translated after delivery to a cell, and this translation provides an RNA-dependent RNA polymerase which then produces both antisense and sense transcripts from the delivered RNA. Thus the delivered RNA can lead to the production of multiple daughter RNAs. These daughter RNAs, as well as collinear subgenomic transcripts, may be translated themselves to provide in situ expression of an encoded immunogen, or may be transcribed to provide further transcripts with the same sense as the delivered RNA which are translated to provide in situ expression of the immunogen. The overall results of this sequence of transcriptions is a large amplification in the number of the introduced replicon RNAs and so the encoded immunogen becomes a major polypeptide product of the cells.

One suitable system for achieving self-replication in this manner is to use an alphavirus-based replicon. These replicons can be +-stranded RNAs which lead to translation of a replicase (or replicase-transcriptase) after delivery to a cell. The replicase can be translated as a polyprotein which auto-cleaves to provide a replication complex which creates genomic −-strand copies of the +-strand delivered RNA. These −-strand transcripts can themselves be transcribed to give further copies of the +-stranded parent RNA and also to give a subgenomic transcript which encodes the immunogen. Translation of the subgenomic transcript can thus lead to in situ expression of the immunogen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons.

A self-replicating RNA molecule can encode (i) an RNA-dependent RNA polymerase which can transcribe RNA from the self-replicating RNA molecule and (ii) an immunogen. The polymerase can be an alphavirus replicase e.g. comprising one or more of alphavirus proteins nsp1, nsp2, nsp3 and nsp4.

Whereas natural alphavirus genomes encode structural virion proteins in addition to the non-structural replicase polyprotein, a self-replicating RNA molecule described herein can lack one or more or all alphavirus structural proteins. Thus a self-replicating RNA can lead to the production of genomic RNA copies of itself in a cell, but not to the production of RNA-containing virions. The inability to produce these virions means that, unlike a wild-type alphavirus, the self-replicating RNA molecule generally does not perpetuate itself in infectious form. The alphavirus structural proteins which are used for perpetuation in wild-type viruses are typically absent from self-replicating RNAs described herein and their place is taken by gene(s) encoding the immunogen of interest, such that the subgenomic transcript encodes the immunogen rather than the structural alphavirus virion proteins.

Thus a self-replicating RNA molecule may have two open reading frames. The first (5′) open reading frame encodes a replicase; the second (3′) open reading frame encodes an immunogen. In some embodiments the RNA may have additional (e.g. downstream) open reading frames e.g. to encode further immunogens (see below) or to encode accessory polypeptides.

Self-replicating RNA molecules can have various lengths but they are typically 5,000-25,000 nucleotides long, e.g., 8,000-15,000 nucleotides, or 9,000-12,000 nucleotides.

A self-replicating RNA molecule described herein may have a 5′ cap (e.g. a 7-methylguanosine). This cap can enhance in vivo translation of the RNA. The 5′ nucleotide of a RNA molecule useful with the present disclosure may have a 5′ triphosphate group. In a capped RNA this may be linked to a 7-methylguanosine via a 5′-to-5′ bridge. In some embodiments, the RNA cap includes mRNA caps. In some embodiments, the mRNA cap may include m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-, where Nm denotes any nucleotide with a 2′ O methylation.

A self-replicating RNA molecule may have a 3′ poly-A tail. It may also include a poly-A polymerase recognition sequence (e.g. AAUAAA) near its 3′ end.

In some embodiments, the self-replicating RNA described herein comprises an elongated polyA tail. In some embodiments, the self-replicating RNA described herein comprises a polyA tail between 30-100 nucleotides in length. In some embodiments, the self-replicating RNA described herein comprises a polyA tail between 35-100 nucleotides in length. In some embodiments, the self-replicating RNA described herein comprises a polyA tail between 40-100 nucleotides in length. In some embodiments, the self-replicating RNA described herein comprises a polyA tail between 45-100 nucleotides in length. In some embodiments, the self-replicating RNA described herein comprises a polyA tail between 50-100 nucleotides in length. In some embodiments, the self-replicating RNA described herein comprises a polyA tail between 55-100 nucleotides in length. In some embodiments, the self-replicating RNA described herein comprises a polyA tail between 60-100 nucleotides in length. In some embodiments, the self-replicating RNA described herein comprises a polyA tail between 65-100 nucleotides in length. In some embodiments, the self-replicating RNA described herein comprises a polyA tail between 70-100 nucleotides in length. In some embodiments, the self-replicating RNA described herein comprises a polyA tail between 80-100 nucleotides in length. In some embodiments, the self-replicating RNA described herein comprises a polyA tail between 90-100 nucleotides in length. In some embodiments, the self-replicating RNA described herein comprises a polyA tail between 95-100 nucleotides in length. In some embodiments, the self-replicating RNA described herein comprises a polyA tail between 30-95 nucleotides in length. In some embodiments, the self-replicating RNA described herein comprises a polyA tail between 30-90 nucleotides in length. In some embodiments, the self-replicating RNA described herein comprises a polyA tail between 30-85 nucleotides in length. In some embodiments, the self-replicating RNA described herein comprises a polyA tail between 30-80 nucleotides in length. In some embodiments, the self-replicating RNA described herein comprises a polyA tail between 30-75 nucleotides in length. In some embodiments, the self-replicating RNA described herein comprises a polyA tail between 30-70 nucleotides in length. In some embodiments, the self-replicating RNA described herein comprises a polyA tail between 30-65 nucleotides in length. In some embodiments, the self-replicating RNA described herein comprises a polyA tail between 30-60 nucleotides in length. In some embodiments, the self-replicating RNA described herein comprises a polyA tail between 30-55 nucleotides in length. In some embodiments, the self-replicating RNA described herein comprises a polyA tail between 30-50 nucleotides in length. In some embodiments, the self-replicating RNA described herein comprises a polyA tail between 30-45 nucleotides in length. In some embodiments, the self-replicating RNA described herein comprises a polyA tail of 10, 15, 20, 25, 30, 35, 40, 45, 50, 55, 60, 65, 70, 75, 80, 85, 90, 95, 100, 110, 120, 130, 140, or more than 140 nucleotides in length.

In some embodiments, the self-replicating RNA described herein has an elongated 3′ UTR. In some embodiments, the self-replicating RNA described herein has a 3′ UTR between 100-500 nucleotides in length. In some embodiments, the self-replicating RNA described herein has a 3′ UTR between 125-500 nucleotides in length. In some embodiments, the self-replicating RNA described herein has a 3′ UTR between 150-500 nucleotides in length. In some embodiments, the self-replicating RNA described herein has a 3′ UTR between 175-500 nucleotides in length. In some embodiments, the self-replicating RNA described herein has a 3′ UTR between 200-500 nucleotides in length. In some embodiments, the self-replicating RNA described herein has a 3′ UTR between 225-500 nucleotides in length. In some embodiments, the self-replicating RNA described herein has a 3′ UTR between 250-500 nucleotides in length. In some embodiments, the self-replicating RNA described herein has a 3′ UTR between 275-500 nucleotides in length. In some embodiments, the self-replicating RNA described herein has a 3′ UTR between 300-500 nucleotides in length. In some embodiments, the self-replicating RNA described herein has a 3′ UTR between 325-500 nucleotides in length. In some embodiments, the self-replicating RNA described herein has a 3′ UTR between 100-476 nucleotides in length. In some embodiments, the self-replicating RNA described herein has a 3′ UTR between 100-450 nucleotides in length. In some embodiments, the self-replicating RNA described herein has a 3′ UTR between 100-425 nucleotides in length. In some embodiments, the self-replicating RNA described herein has a 3′ UTR between 100-400 nucleotides in length. In some embodiments, the self-replicating RNA described herein has a 3′ UTR between 100-375 nucleotides in length. In some embodiments, the self-replicating RNA described herein has a 3′ UTR between 100-350 nucleotides in length. In some embodiments, the self-replicating RNA described herein has a 3′ UTR between 300-350 nucleotides in length. In some embodiments, the self-replicating RNA described herein has a 3′ UTR about 330 nucleotides in length. In some embodiments, the self-replicating RNA described herein comprises a 3′ UTR of 50, 75, 100, 125, 150, 175, 200, 225, 250, 275, 300, 325, 350, 375, 400 or more than 440 nucleotides in length.

In some embodiments, the self-replicating RNA molecule described herein comprises a Cap (e.g., m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm—where Nm denotes any nucleotide with a 2′ O methylation), a 5′ UTR, one or more alphavirus proteins (e.g., nsp1, nsp2, nsp3 and nsp4), a gene of interest (e.g., IL-12), a 3′ UTR, and a poly A tail. In some embodiments, the 3′ UTR is an elongated UTR comprising about 50, 75, 100, 125, 150, 175, 200, 225, 250, 275, 300, 325, 350, 375, 400 or more than 440 nucleotides in length. In some embodiments, the 3′ UTR comprises about 330 nucleotides in length. In some embodiments, the polyA tail comprises about 10, 15, 20, 25, 30, 35, 40, 45, 50, 55, 60, 65, 70, 75, 80, 85, 90, 95, 100, 110, 120, 130, 140, or more than 140 nucleotides in length. In some embodiments, the polyA tail comprises about 65 nucleotides in length.

In some embodiments, the self-replicating RNA molecule described herein comprises Cap 0, a 5′ UTR, one or more alphavirus proteins (e.g., nsp1, nsp2, nsp3 and nsp4), a gene of interest (e.g., IL-12), a 3′ UTR comprising about 100-400 (e.g., 330 nucleotides) nucleotides in length, and a poly A tail comprising about 40 to about 100 nucleotides in length (e.g., 40, 65, or 95 nucleotides in length). In some embodiments, the self-replicating RNA molecule described herein comprises Cap 1, a 5′ UTR, one or more alphavirus proteins (e.g., nsp1, nsp2, nsp3 and nsp4), a gene of interest (e.g., IL-12), a 3′ UTR comprising about 100-400 (e.g., 330 nucleotides) nucleotides in length, and a poly A tail comprising about 40 to about 100 nucleotides in length (e.g., 40, 65, or 95 nucleotides in length).

A self-replicating RNA molecule will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

A self-replicating RNA molecule described herein can be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these are matched with the encoded replicase, to ensure that the IVT-transcribed RNA can function efficiently as a substrate for its self-encoded replicase.

### IL-12 and Innate Immune Response

Self-replicating RNA molecules described herein can encode an IL-12 polypeptide. In some embodiments, after administration of the RNA, the immunogen is translated in vivo and can elicit an immune response in the recipient. In some embodiments, the immunogen may elicit a native immune response. In some embodiments, the innate immune response may comprise an antibody response. In some embodiments, the immune response may comprise CD8+ T cells. In some embodiments, The immune response may be linked to antitumor effects.

In some embodiments, there is a method of enhancing an immune response, comprising administering the composition of any embodiment to the subject.

In some embodiments, there is a method of treating a tumor in a subject, comprising administering the composition of an embodiment to the subject.

### Methods of Treatment

Provided herein are compositions (e.g., pharmaceutical compositions), methods, kits and reagents for the administration of liposome packaged RNA replicons encoding IL-12 to elicit an innate immune response having antitumor effects in humans and other mammals. Theses compositions can be used as therapeutic agents.

The term “dose” as used herein in reference to an immunogenic composition refers to a measured portion of the immunogenic composition taken by (administered to or received by) a subject at any one time.

The disclosure further includes a liposome packaged RNA replicons encoding IL-12 administered in combination with one or more anti-cancer agents or uses of the composition in combination with one or more anti-cancer agents to the subject. In one embodiment, the combination therapy can be a combination of the liposome packaged RNA replicons encoding IL-12 and one or more standard therapy. The additional anti-cancer agents can be a protein, e.g., an antibody, or a polynucleotide, e.g., mRNA. In some embodiments, the anti-cancer agents are a protein, e.g., an antibody.

In some embodiments, the one or more anti-cancer agents are an approved agent by the United States Food and Drug Administration. In other embodiments, the one or more anti-cancer agents are a pre-approved agent by the United States Food and Drug Administration

Alternative embodiments of the present disclosure include a combination therapy of IL12 and any other agents, e.g., an anti-PD-1 antibody, an anti-PD-L1 antibody.

A method of eliciting an immune response in a subject after administration of the liposome packaged RNA replicon encoding IL-12 is provided in aspects of the present disclosure. The method involves administering to the subject a liposome packaged RNA replicon encoding IL-12 comprising at least one srRNA polynucleotide having an open reading frame encoding IL-12, thereby stimulating an innate immunity response that is linked to antitumor effects. An “anti-antigenic polypeptide antibody” is a serum antibody the binds specifically to the antigenic polypeptide.

A method of eliciting an immune response in a subject is provided in aspects of the present disclosure. The method involves administering to the subject a composition comprising a liposome-packaged RNA replicon encoding IL-12 comprising at least one RNA polynucleotide having an open reading frame encoding IL-12.

In some embodiments, the composition is administered to the subject intratumorally or intramuscularly.

In some embodiments, the composition is administered to the subject at least three times. In some embodiments, the composition is administered to the subject three times. In some embodiments, the composition is administered to the subject four times. In some embodiments, the composition is administered to the subject five times. In some embodiments, the composition is administered to the subject six times. In some embodiments, the composition is administered to the subject seven times. In some embodiments, the composition is administered to the subject eight times. In some embodiments, the composition is administered to the subject nine times. In some embodiments, the composition is administered to the subject ten times.

In some embodiments, the composition is administered once every one, two, three or four weeks. In some embodiments, the composition is administered once every week. In some embodiments, the composition is administered once every two weeks. In some embodiments, the composition is administered once every three weeks. In some embodiments, the composition is administered once every four weeks.

In some embodiments, the composition is administered to the subject at a dose of 0.1-200 μg In some embodiments, the composition is administered to the subject at a dose of 0.1-100 μg. In some embodiments, the composition is administered to the subject at a dose of 0.1-90 μg. In some embodiments, the composition is administered to the subject at a dose of 0.1-80 μg. In some embodiments, the composition is administered to the subject at a dose of 0.1-70 μg. In some embodiments, the composition is administered to the subject at a dose of 0.1-60 μg. In some embodiments, the composition is administered to the subject at a dose of 0.1-50 μg. In some embodiments, the composition is administered to the subject at a dose of 0.1-40 μg. In some embodiments, the composition is administered to the subject at a dose of 0.1-30 μg. In some embodiments, the composition is administered to the subject at a dose of 0.1-20 μg. In some embodiments, the composition is administered to the subject at a dose of 0.1-15 μg. In some embodiments, the composition is administered to the subject at a dose of 0.1-10 μg. In some embodiments, the composition is administered to the subject at a dose of 0.1-8 μg. In some embodiments, the composition is administered to the subject at a dose of 0.1-6 μg. In some embodiments, the composition is administered to the subject at a dose of 0.1-5 μg. In some embodiments, the composition is administered to the subject at a dose of 0.1-4 μg. In some embodiments, the composition is administered to the subject at a dose of 0.1-3 μg. In some embodiments, the composition is administered to the subject at a dose of 0.1-2.5 μg. In some embodiments, the composition is administered to the subject at a dose of 0.1-2 μg. In some embodiments, the composition is administered to the subject at a dose of 0.1-1 μg.

In some embodiments, the composition enhances an immune response in the subject following administration.

In some embodiments, there is a method using any of the above methods, further comprising the administration of a checkpoint inhibitor, optionally wherein the checkpoint inhibitor is administered prior to, concurrent with, or following administration of the srRNA or the composition.

In some embodiments, the checkpoint inhibitor is a PD-1, PD-L1 inhibitor, or a combination thereof.

In some embodiments, the PD-1 inhibitor is selected from the group comprising small molecule PD-1 inhibitor, anti-PD-1 antibody, or a combination thereof. In some embodiments, the PD-L1 inhibitor is selected from the group comprising small molecule PD-L1 inhibitor, anti-PD-L1 antibody, or a combination thereof.

In some embodiments, the checkpoint inhibitor is an anti-PD-1 antibody, an anti-PD-L1 antibody, or a combination thereof.

In some embodiments, the anti-PD-1 antibody is selected from the group comprising anti-PD-1 antibody, Pembrolizumab (Keytruda), Nivolumab (Opdivo), and Cemiplimab (Libtayo). In some embodiments, the anti-PD-L1 antibody is selected from the group comprising anti-PD-L1 antibody, Atezolizumab (Tecentriq), Avelumab (Bavencio), and Durvalumab (Imfinzi).

In some embodiments, the anti-PD-1 antibody, anti-PD-L1 antibody, or a combination thereof is administered to the subject at a dose of 1-20 mg/kg. In some embodiments, the anti-PD-1 antibody, anti-PD-L1 antibody, or a combination thereof is administered to the subject at a dose of 1-15 mg/kg. In some embodiments, the anti-PD-1 antibody, anti-PD-L1 antibody, or a combination thereof is administered to the subject at a dose of 1-10 mg/kg. In some embodiments, the anti-PD-1 antibody, anti-PD-L1 antibody, or a combination thereof is administered to the subject at a dose of 1-5 mg/kg. In some embodiments, the anti-PD-1 antibody, anti-PD-L1 antibody, or a combination thereof is administered to the subject at a dose of 1-4 mg/kg. In some embodiments, the anti-PD-1 antibody, anti-PD-L1 antibody, or a combination thereof is administered to the subject at a dose of 1-3 mg/kg. In some embodiments, the anti-PD-1 antibody, anti-PD-L1 antibody, or a combination thereof is administered to the subject at a dose of 1-2 mg/kg.

In some embodiments, the anti-PD-1 antibody, anti-PD-L1 antibody, or a combination thereof is administered to the subject at a dose of about 5 mg/kg. In some embodiments, the anti-PD-1 antibody, anti-PD-L1 antibody, or a combination thereof is administered to the subject at a dose of about 4 mg/kg. In some embodiments, the anti-PD-1 antibody, anti-PD-L1 antibody, or a combination thereof is administered to the subject at a dose of about 3 mg/kg. In some embodiments, the anti-PD-1 antibody, anti-PD-L1 antibody, or a combination thereof is administered to the subject at a dose of about 2 mg/kg.

In some embodiments, the compositions described herein comprising liposome packaged RNA replicons encoding IL-12 results in reduced tumor size, increased survival, or both. In some embodiments, tumor size decreases by at least about 10% to about 25%, about 10% to about 50%, about 20% to about 100%. In some embodiments, tumor size decreases by at least 5%, at least 10%, at least 20%, at least 30%, at least 40%, at least 50%, at least 60%, at least 70%, at least 80%, at least 90%, at least 95%, or greater than 95%. Tumor size in some cases decreases by 5-95%, 10-90%, 20-80%, 30-70%, 40-60%, 50-95%, 65-85%, or 75-95%. In some cases, the decrease in tumor size is by at least 5%, 10%, 15%, 20%, 25%, 30%, 35%, 40%, 45%, 50%, 55%, 60%, 65%, 70%, 75%, 80%, 85%, 90%, 95%, or greater than 95%. The decrease in tumor size sometimes is at least 5%. In some cases, the decrease in tumor size is by at least 10%. The decrease in tumor size in some cases is at least 30%. In some instances, the decrease in tumor size may be by at least 50%. In some embodiments, survival increases by 5-95%, 10-90%, 20-80%, 30-70%, 40-60%, 50-95%, 65-85%, or 75-95%. In some embodiments, survival increases by at least 5%, 10%, 15%, 20%, 25%, 30%, 35%, 40%.

In some embodiments, the compositions described herein are administered with an anti-PD-1 antibody, anti-PD-L1 antibody, or a combination thereof, and results in a synergistic effect. In some embodiments, the compositions described herein are administered with an anti-PD-1 antibody, anti-PD-L1 antibody, or a combination thereof and results in reduced tumor size, increased survival, or both. In some embodiments, tumor size decreases by at least about 10% to about 25%, about 10% to about 50%, about 20% to about 100%. In some embodiments, tumor size decreases by at least 5%, at least 10%, at least 20%, at least 30%, at least 40%, at least 50%, at least 60%, at least 70%, at least 80%, at least 90%, at least 95%, or greater than 95%. Tumor size in some cases decreases by 5-95%, 10-90%, 20-80%, 30-70%, 40-60%, 50-95%, 65-85%, or 75-95%. In some cases, the decrease in tumor size is by at least 5%, 10%, 15%, 20%, 25%, 30%, 35%, 40%, 45%, 50%, 55%, 60%, 65%, 70%, 75%, 80%, 85%, 90%, 95%, or greater than 95%. The decrease in tumor size sometimes is at least 5%. In some cases, the decrease in tumor size is by at least 10%. The decrease in tumor size in some cases is at least 30%. In some instances, the decrease in tumor size may be by at least 50%. In some embodiments, survival increases by 5-95%, 10-90%, 20-80%, 30-70%, 40-60%, 50-95%, 65-85%, or 75-95%. In some embodiments, survival increases by at least 5%, 10%, 15%, 20%, 25%, 30%, 35%, 40%.

### Modes of Administration

A liposome packaged RNA replicon encoding IL-12 may be administered to a subject, e.g., intratumorally or intramuscularly. In some embodiments, the liposome packaged RNA replicon encoding IL-12 is administered intravenously. The liposome packaged RNA replicon encoding IL-12 may be administered to the subject at a dose of 5-200 μg. The liposome packaged RNA replicon encoding IL-12 may be administered (e.g., intravenously) to the subject at least three times. In some embodiments, the liposome packaged RNA replicon encoding IL-12 is administered (e.g., intravenously) to the subject at least three times biweekly. The liposome packaged RNA replicon encoding IL-12 may be administered once every one, two, three or four weeks.

### Kits

The present disclosure also provides kits comprising: i) a lyophilized srRNA comprising Il-12; ii) a delivery vehicle, such as an LNP; iii) instructions for mixing the first composition with the second composition to prepare an immunogenic composition; and iv) a set of instructions for administration of the immunogenic composition to stimulate an innate immune response in a mammalian subject, such as a human subject in need thereof.

In some embodiments, the srRNA is lyophilized.

In some embodiments, the lyophilized srRNA is at a temperature at or below 22° C., optionally about 2-8° C.

### EXAMPLES

### Example 1. Interleukin-12 Self-Replicating RNA

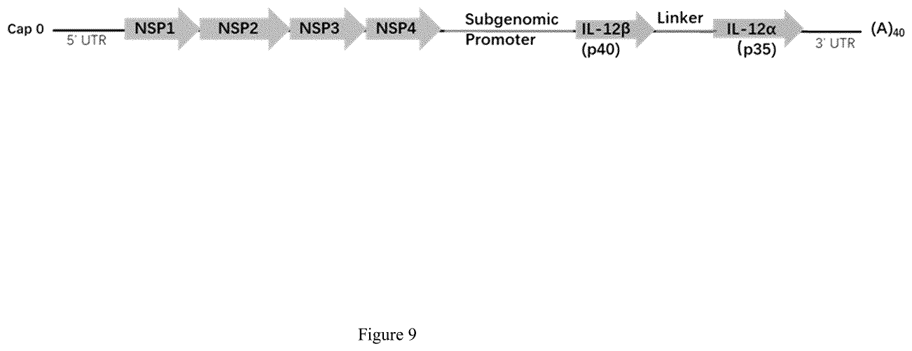

The target drug is a RNA replicon comprising a self-replicating RNA (srRNA) that encodes interleukin (IL)-12. An exemplary structure is illustrated in FIG. 9. The srRNA construct includes a sequence encoding IL-12 encoded by the sequence shown in SEQ ID NO: 1.

### Example 2. RNA In Vitro Transcription and Capping

The RNA replicon was based on an engineered alphavirus genome containing the genes encoding the non-structural proteins which allow RNA replication, whereas the structural protein sequences was replaced with the gene sequence of IL-12. The RNA replicon comprises a 5′ cap untranslated region (UTR), four non-structural genes (nsp1-4), a 26S subgenomic promoter, IL-12 gene, and a 3′ terminal polyadenylated tail. To generate linear templates for RNA transcription, plasmid DNA was cut by restriction digest using BspQI enzyme (New England Biolabs, R0712L), and purified using PureLink® PCR Purification Kit (Invitrogen, K310002). RNA was transcribed using 5000 U/ml T7 polymerase (New England Biolabs, M0251L), 1000 U/ml RNase inhibitor (New England Biolabs, M0314L), 2 U/ml pyrophosphatase (New England Biolabs, M2403L), 2 mM Spermidine (Sigma, 5292), 10 mM DL-Dithiothreitol (Sigma, 43816), 6 mM rNTP (New England Biolabs, N0466S), 24 mM MgCl2 (Invitrogen, AM9530G) and 40 mM Tris-HCl (Sigma, T2694). The mixture was incubated and shaking at 37° C. for 3 hours. RNA transcripts were capped with vaccinia capping enzyme (New England Biolabs, M2080S) using GTP (Invitrogen, R0461) and S-adenosyl-methionine (New England Biolabs, B9003S) as substrates to create a Cap 0 structure. RNA was purified using LiCl precipitation.

### Example 3. Formulation Production

Lipid nanoparticles were formulated by rapid mixing of ethanol phase and aqueous phase using the microfluidic device (INano™ L system, Micro&Nano). The aqueous phase is a citrate buffer containing the purified RNA replicon. The ethanol phase comprises a propriety ionizable lipid 1,2-Diastearoyl-sn-glycero-3-phosphocholine (DSPC) (Avanti, 850365P), Cholesterol (Sigma-Aldrich, C8667) and 1,2-dimyristoyl-sn-glycero-3-phosphoethanolamine-N-[methoxy(polyethylene glycol)-2000] (NOF, GM020). The RNA replicon-LNPs were assembled with the mole ratios 10:48:2:40 (DSPC:cholesterol:PEG 2000:ionizable lipid) at N/P lipid:RNA ratio 8. Formulations were characterized for particle size, RNA concentration, encapsulation efficiency, and ability to protect from RNase digestion.

### Example 4. Cell Lines and Animals

MC38 tumor cells (NTCC-MC38) were maintained in vitro as a monolayer culture in DMEM+2 mM glutamine supplemented with 10% heat inactivated fetal bovine serum, 100 U/ml penicillin and 100 μg/ml streptomycin at 37° C. in an atmosphere of 5% CO2 in air. The tumor cells were routinely subcultured twice weekly by trypsin-EDTA treatment. The cells growing in exponential growth phase were harvested and counted for tumor inoculation. Female C57BL/6 mice at 6-8 weeks of age were purchased from the Shanghai Lingchang Biological Technology.

All procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured three times weekly), eye/hair matting and any other abnormal effect as stated in the protocol. Death and observed clinical signs were recorded on the basis of the numbers of animals within each subset.

### Example 5. Tumor Inoculation and Tumor Therapy

Each mouse was inoculated subcutaneously at the right flank with MC38 tumor cells (0.3×106) in 0.1 ml of PBS for tumor development. Treatments were started on day 8 after tumor inoculation when the average tumor size reached app 80 mm³. The animals were assigned into groups using an Excel-based randomization software program performing stratified randomization based upon tumor volumes.

For the distal tumor experiment, each mouse was inoculated subcutaneously at the right upper flank with MC38 tumor cells (0.3×106) in 0.1 ml of PBS and at the left upper flank with MC38 tumor cells (0.1×106) in 0.1 ml of PBS for tumor development. Treatments were started on day 8 after tumor inoculation when the right-side average tumor size reached 81 mm³. The animals were assigned into groups using an Excel-based randomization software program performing stratified randomization based upon tumor volumes.

After the animals were grouped, the animals were injected intratumorally with PBS(control) or target drug in ˜100 μl of PBS as indicated.

### Example 6. Antibodies, Staining and FACS Analysis

Antibodies used are seen in Table 1 below.

TABLE 1
Antibodies

| # | Antibody | Fluorochrome | Supplier | Cat# |
|---|---|---|---|---|
| 1 | CD45 | BUV395 | BD | 564279 |
| 2 | Live/Dead | BV421 | Invitrogen | L34964 |
| 3 | CD8 | BV510 | BD | 563068 |
| 4 | Ly6C | BV711 | BioLegend | 128037 |
| 5 | CD3 | FITC | BD | 561798 |
| 6 | CD4 | PerCP-Cy5.5 | BioLegend | 100434 |
| 7 | PD-1 | PE | BioLegend | 114118 |
| 8 | CD335 | PE-CF594 | BloLegend | 137630 |
| 9 | Ly6G | APC | BD | 560599 |
| 10 | CD11b | AF700 | BD | 557960 |

CryopreservTumor-bearing mice were killed and fresh tumor samples from each mouse was minced individually and digested with mixed enzymes in C tubes. C tubes were attached onto the sleeves of the Gentle MACS Dissociator before running the program “m_imptumor_01_01” one time. C tubes were then incubated for 30 minutes at 37° C., followed by another round of program “m_imptumor_01_01”. Digested tissues were filtered through 70 μm cell strainers. Cells were washed twice with DPBS before staining.

100 μL resuspended tumor cells (10 million/mL) per test were seeded on 96 V-hole plate. After centrifugation, cells were suspended with 100 μL DPBS. BV421 live/dead (0.1 μL per well) was added and incubated for 30 min at 4° C. in dark. Cells were washed twice and suspended with 100 μL staining buffer. Anti-mouse CD16/CD32 (1 μL per well) was added and incubated for 5 min at 4° C. in the dark. Appropriate antibody was added into the cell suspension and incubated for 30 min at 4° C. in the dark. Cells were washed twice and suspended with 200 μL staining buffer. Transferred cell suspensions from 96-well plates to tube for Flow Cytometer detection.

Stained cells were analyzed by BD Fortessa X20 Flow Cytometer. PMN-MDSC population was set as stopping gate. 3,000-5,000 cells of PMN-MDSC were collected.

### Example 7. ELISA Analysis

First, ˜50 mg tumor tissues were lysed with RIPA Buffer (contains 1% Protease Inhibitor Cocktail and 1% Phosphatase Inhibitor Cocktail 2) to collect the supernatant protein, Measure protein concentrations by Pierce™ BCA Protein Assay Kit.

Then, IL-12 concentration in Serum and Tumor protein were detected according to the procedure of R&D Quantikine ELISA Mouse IL-12 p70 Immunoassay Kit (R&D-SM1270).

### Example 8. QPCR Analysis

QPCR analysis was performed. First, ˜60 mg piece of tissue was cut with a scalpel (a few grams) and 0.5 g tissue was transferred to a 1.5 mL homogenizer tube e.g. BeadBeater tube (pre-loaded with glass beads) with appropriate amount of tissue lysis buffer on wet ice.

Blood RNA were extracted according to the procedure of QIAamp RNA Blood Mini Kit (Qiagen-52304), Tissue RNA were extracted according to the procedure of RNeasy Mini Kit (Qiagen-74104). RNA concentration was measured using Nanodrop 1000 (Thermo Sci.).

Reverse transcription: the Reaction Mix as follow was prepared comprising total volume is 20 μL and 2 μg RNA samples.

TABLE 2
Reaction Mixture

| Component | Volume/reaction (MI) |
|---|---|
| 10x RT buffer | 2.0 |
| 25x DNTP Mix (100 Mm) | 0.8 |
| 10X RT Random Primers | 2.0 |
| MultiScribe Reverse transcriptase | 1.0 |
| Nuclease-free H₂O | 3.2 (adjusted) |
| RNA | 10 (adjusted) |
| RNAse inhibitor | 1.0 |
| Total per reaction | 20.0 |

Reverse transcription reaction was performed according to Table 3 below.

TABLE 3
Reverse Transcription Reaction

| | Step 1 | Step 2 | Step 3 | Step 4 |
|---|---|---|---|---|
| Temperature (° C.) | 25 | 37 | 85 | 4 |
| Time | 10 minutes | 120 minutes | 5 minutes | Indefinite |

qPCR analysis of mRNA expression level of NSP4 gene using SYBR Green was performed. The qPCR reaction system was prepared as in Table 4 below including a total volume of 10 μL, including 100-200 ng cDNA template.

TABLE 4
qPCR Analysis

| | |
|---|---|
| Template | 1 μL |
| Power SYBR Green Master Mix | 5 μL |
| Forward primer | 0.25 μL |
| GTCCAGGAAGGTGGAGAACA (5 uμM) | |
| Reverse primer | 0.25 μL |
| AAGGCACGGTTCACACTAGA (5 μM) | |
| H₂O | 3.5 μL |
| Total volume | 10 μL |

qPCR reaction was performed on a real-time PCR machine according to Table 5 below.

TABLE 5
qPCR Reaction

| Temperature (° C.) | Time (sec) | Cycles |
|---|---|---|
| 95 | 600 | 1 |
| 95 | 15 | 40 |
| 60 | 60 | |

### Example 9. Data Analysis

Statistical analysis of difference in the tumor volume among the groups were conducted on the data obtained at the 15th day after treatment. A one-way ANOVA was performed to compare the tumor volume, and a significant F-statistics (a ratio of treatment variance to the error variance) was obtained, thus comparisons between groups were carried out with Games-Howell test. All data were analyzed using SPSS 17.0. p<0.05 was considered to be statistically significant.

### Example 10. Efficacy of Target Drug in MC38 Mouse Model

In this study, the therapeutic efficacy of Liposome-packaged RNA replicon as a single agent in the treatment of the murine MC38 tumor model in C57BL/6 mice was evaluated. The mean tumor size of the vehicle control group reached 2,470 mm³ on Day 15 after treatment. Treatment with target drug at dose levels of 1 μg/mouse, 2.5 μg/mouse, 5 μg/mouse, and 10 μg/mouse demonstrated significant antitumor activity. The Tumor Growth Inhibitions (TGIs) were calculated. The mean tumor sizes were 464 mm³, 195 mm³, 343 mm³, and 160 mm³, respectively, in each group on Day 15 (T/C value=18.74%, 7.88%, 13.88%, and 6.47%; TGI=83.95%, 95.20%, 88.99% and 96.65%; p=0.002, 0.002, 0.002, and 0.001 compared with the vehicle group) (FIG. 1).

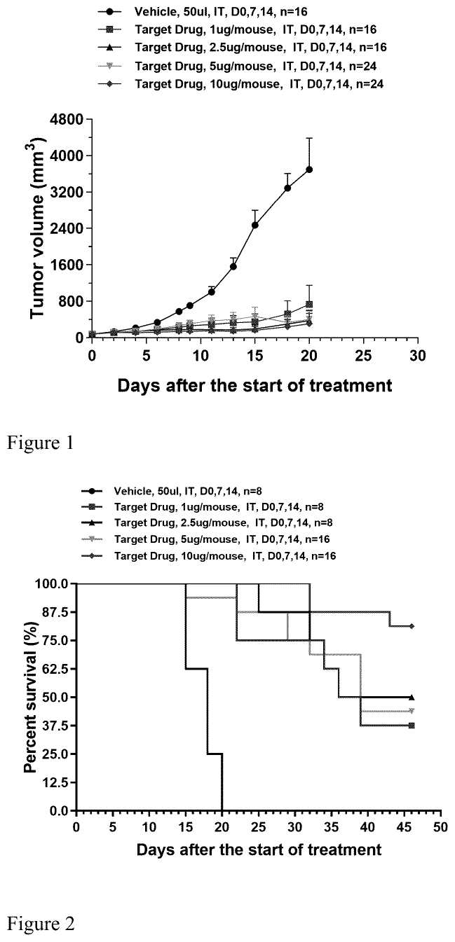

The survival of tumor-bearing mice is shown in FIG. 2. All mice in the vehicle group were euthanized on Day 20, as the tumor size exceeded 3,000 mm³. On Day 46 after treatment, the overall survival rates in dose groups of 1 μg/mouse, 2.5 μg/mouse, 5 μg/mouse, and 10 μg/mouse target drug were 37.5%, 50%, 43%, and 82%, respectively. Treatment with target drug substantially reduced the tumor growth and prolonged the survival of tumor-bearing mice.

On Day 46 after the start of treatment, 25 mice with a regressed tumor were re-challenged subcutaneously at the left upper flank with MC38 (0.3×106) in 0.1 mL of PBS for tumor development. No additional test article treatment was administered for the re-challenge. Eight naive mice were challenged with the same number of MC38 cells as control.

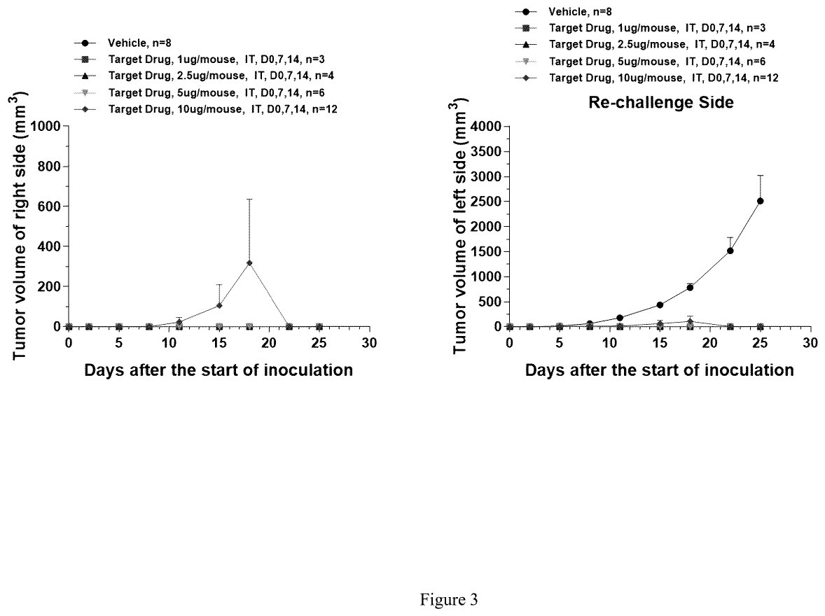

When the mice with complete tumor elimination were re-challenged with MC38 cancer cells on Day 46, the re-challenged tumor cells did not grow, except for 1 mouse (the tumor cells in both the original side (right side) and re-challenge side (left side) were grown) in the target drug 10 μg/mouse group (FIG. 3).

After a re-challenge with MC38, 96.6% (24/25) of mice in the target drug groups remained tumor free. In comparison, 100% of the mice in the control group developed tumors.

In summary, the target drug as a single agent at dose levels of 1 μg/mouse, 2.5 μg/mouse, 5 μg/mouse and 10 μg/mouse produced significant anti-tumor activity against the MC38 colon cancer syngeneic model in this study.

### Example 11. Concentration of IL-12 in Tumor and Serum on Day 10 (3 Days after Second Injection) after Treatment

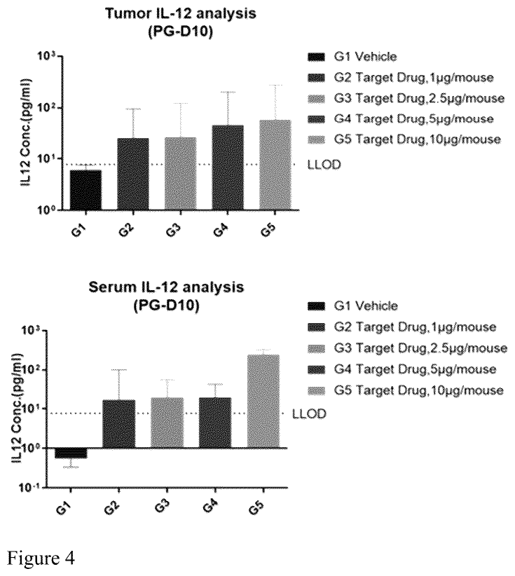

To further evaluate the effect of target drug on the production of IL-12 in tumor/blood, tumor/serum were collected at day 10 after the start of treatment and were analyzed by ELISA (FIG. 4).

Compared with vehicle group, IL-12 production in tumor increased in all dose groups treated with target drug (Group 2, 3, 4, 5) on Day 10. In blood, the IL-12 production also showed a marginal increase in the 1 μg, 2.5 μg, and 5 μg groups, with the expressed IL-12 level around the lower limit of detection (LLOD). Only the group treated with 10 μg/mouse of target drug showed a considerable increase in the amount of IL-12 production in blood.

### Example 12. Target Drug Remodels the TME (Tumor Microenvironment)

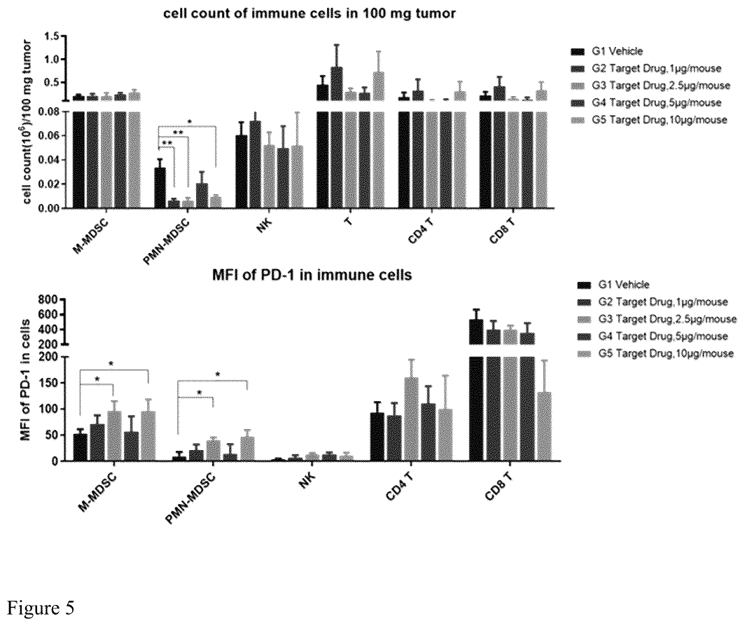

To evaluate the effect of target drug on the quantities of different immune cells in tumor, including M-MDSC (monocytic-MDSC), PMN-MDSC (polymorphonuclear-MDSC), CD4 T cells, CD8 T cells, natural killer (NK) cells, and the expression of PD-1, MC38 tumors were collected on Day 10 (i.e., 3 days after the second dose) and were analyzed by flow cytometry (FIG. 5).

Compared with vehicle control group, the cell count of PMN-MDSC per 100 mg tumor was significantly decreased in groups treated with 1 μg/mouse, 2.5 μg/mouse, and 10 μg/mouse target drug (Groups 2, 3, and 5). The cell count of both CD4 T and CD8 T cells showed a trend of increase in the 1 μg/mouse and 10 μg/mouse groups (Groups 2, 5). The expression of PD-1 in M-MDSC and PAN-MDSC was significantly increased in the groups treated with 2.5 μg/mouse or 10 μg/mouse target drug.

In conclusion, the test article target drug at dose levels of 1, 2.5, 5, and 10 μg/mouse demonstrated significant antitumor activity in the murine syngeneic MC38 colon cancer model, leading to tumor regression and improved survival of tumor bearing mice. Intratumor administration of target drug resulted in predominantly local IL-12 production in the tumor with no or low IL-12 increase in blood. The antitumor activity was associated with a decrease of myeloid suppressive cells and an increase of T cells in the tumor. Re-challenging the mice with regressed tumors suggested the induction of tumor-specific immune memory.

### Example 13. Effect of Intratumor Target Drug Against a Distal Tumor and Synergy with an Anti-PD-1 Antibody in the MC38 Tumor Model in C57BI Mice

In this study, the therapeutic efficacy of target drug as a single agent was evaluated in the MC38 murine syngeneic tumor model in C57BL/6 mice to treat a directly injected tumor as well as a distal non-injected tumor. In addition, the intratumoral administration of target drug was assessed in combination with a systemic anti-murine PD-1 (anti-mPD-1) antibody in order to explore the effects of a combination treatment relative to the single-agent Target drug treatment.

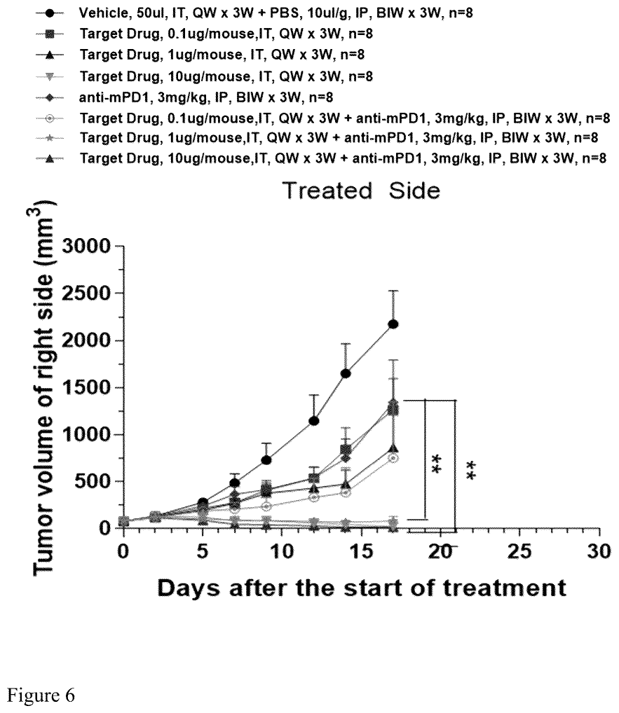

For the right-side tumor (i.e., administration side tumor), the mean tumor size of the vehicle treated control mice reached 2,172 mm³ on Day 14 (see details in FIG. 6 and Table 6 below). The Tumor Growth Inhibitions (TGIs) were calculated. Single treatment with Target drug at dose level of 0.1 μg/mouse and 1 μg/mouse produced a moderate antitumor activity, their mean tumor size was 1,267 mm³ and 862 mm³ at the same time (T/C value=51.78% and 28.76%; TGI=51.20% and 75.01%; p=0.495 and 0.103 compared with control group). Single treatment with the target drug at dose level of 10 μg/mouse produced remarkable anti-tumor activity, the mean tumor size was 24 mm³ at the same time (T/C value=2.71%; TGI=102.34%; p=0.025 compared with control group).

Combination treatment of Target drug at dose level of 0.1, 1, and 10 μg/mouse with anti-mPD-1 at dose level of 3 mg/kg further enhanced the antitumor activity. The mean tumor sizes were 754 mm³, 83 mm³ and 13 mm³ at the same time (T/C value=23.15%, 4.18% and 1.06%; TGI=80.87%, 100.80% and 104.05%; p=0.122, 0.027 and 0.023 compared with the vehicle group). Single treatment with anti-mPD-1 at dose level of 3 mg/kg produced a moderate anti-tumor activity similar to that with Target drug at dose level of 0.1 μg/mouse.

TABLE 6
Effect of Intratumor Target Drug Against a Distal Tumor
and Synergy with an Anti-PD-1 Antibody in the MC38
Tumor Model in C57BI Mice (right side, treated side)

| | | Statistical Significance | | |
|---|---|---|---|---|
| | D 14 | (n = 8) | | |
| Group | a - TGI (%) | b | c | d |
| Vehicle | 0 | — | ns | |
| Target Drug 0.1 ug/mouse | 51.20 | ns | ns | |
| Target Drug 1 ug/mouse | 75.01 | ns | ns | |
| Target Drug 10 ug/mouse | 102.34 | * | * | |
| Target Drug 0.1 ug/mouse + | 80.87 | ns | ns | ns |
| anti-PD-1 3 mpk | | | | |
| Target Drug 1 ug/mouse + | 100.80 | * | ns | * |
| anti-PD-1 3 mpk | | | | |
| Target Drug 10 ug/mouse + | 104.05 | * | * | ns |
| anti-PD-1 3 mpk | | | | |
| anti-PD-1 3 mpk | 54.45 | ns | — | |

Note:
a. TGI % = (1 − TRTV/CRTV) × 100% (RTV = Vt/V0);
b. all groups compare to Vehicle at Day 14;
c, all groups compare to anti-PD-1 3 mpk at Day 14;
d. combination therapy compare to monotherapy dose by dose at Day 14,
* p < 0.05;

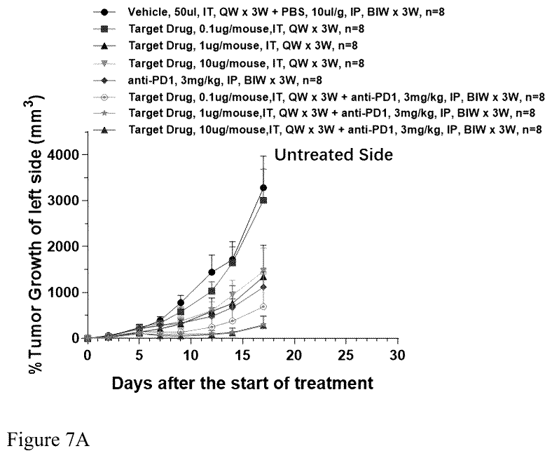

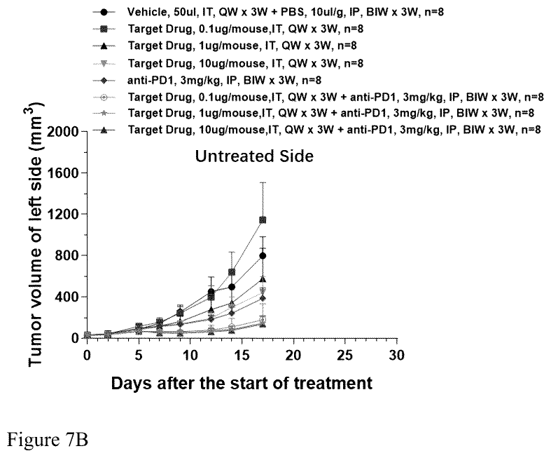

For the left-side tumor (i.e., untreated distal tumor), the mean tumor size of the vehicle treated control mice reached 497 mm³ on Day 14 (see FIGS. 7A and 7B and Table 7 below). Single-agent treatment with Target drug at dose level of 0.1 μg/mouse show no anti-tumor activity (tumor volume=639 mm³; T/C value=113.53%; TGI=−29.47%; p=0.999 compared to the vehicle group). Single treatment with Target drug at dose levels of 1 and 10 μg/mouse produced a minor anti-tumor activity (tumor volume=343 mm³ and 301 mm³; T/C value=56.59% and 60.34%; TGI=34.23% and 41.81%; p=0.996 and 0.955 compared to the vehicle group).

Combination treatment of Target drug at dose level of 0.1, 1, and 10 u g/mouse with anti-mPD-1 at dose level of 3 mg/kg produced substantial anti-tumor activities. The mean tumor sizes were 112 mm³, 85 mm³, and 78 mm³ at the same time (T/C value=25.20%, 15.17% and 14.96%; TGI=81.60%, 88.70% and 89.77%; p=0.442, 0.309 and 0.299 compared to the vehicle group).

TABLE 7
Effect of Intratumor Target Drug Against a Distal Tumor
and Synergy with an Anti-PD-1 Antibody in the MC38 Tumor
Model in C57BI Mice (left side, untreated side)

| | | Statistical Significance | | |
|---|---|---|---|---|
| | D 14 | (n = 8) | | |
| Group | a - TGI (%) | b | c | d |
| Vehicle | 0 | — | ns | — |
| Target Drug 0.1 ug/mouse | −29.47 | ns | ns | — |
| Target Drug 1 ug/mouse | 34.23 | ns | ns | — |
| Target Drug 10 ug/mouse | 41.80 | ns | ns | — |
| Target Drug 0.1 ug/mouse + | 80.87 | ns | ns | * |
| anti-PD-1 3 mpk | | | | |
| Target Drug 1 ug/mouse + | 81.6 | ns | ns | * |
| anti-PD-1 3 mpk | | | | |
| Target Drug 10 ug/mouse + | 88.7 | ns | ns | ns |
| anti-PD-1 3 mpk | | | | |
| anti-PD-1 3 mpk | 51.10 | ns | — | — |

Note:
a. TGI % = (1 − TRTV/CRTV) × 100% (RTV = Vt/V0);
b. all groups compare to Vehicle at Day 14;
c, all groups compare to anti-PD-1 3 mpk at Day 14;
d. combination therapy compare to monotherapy dose by dose at Day 14,
* p < 0.05;

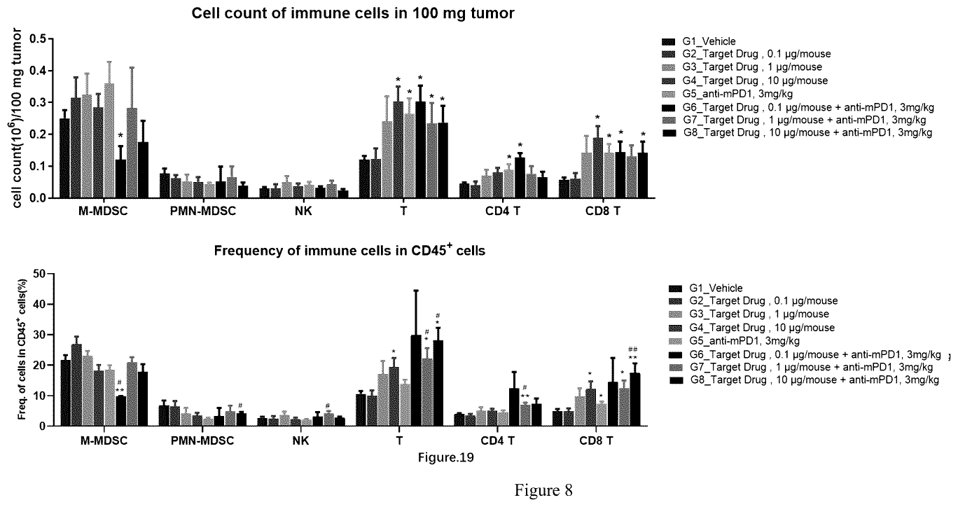

Compared with the vehicle group, a trend of M-MDSC decrease was observed in mice that received the Target drug+anti-mPD1 combination therapy (FIG. 8). The cell count of M MDSC per 100 mg tumor was significantly decreased in the 0.1 μg/mouse Target drug+anti-mPD1 combination group (Group 6). The cell count of T cells, especially CD8 T cells, were significantly increased in the groups treated with high-dose Target drug alone (Group 4), anti-mPD1 alone (Group 5), and combination treatment (Group 8). A similar trend was also found on the frequencies of these immune cells. In addition, the percentages of T cells and CD8 T in Group 8 were significantly higher than those in Group 5.

In conclusion, the test article Target drug as single agent at dose level of 0.1, 1, and 10 u g/mouse exhibited an anti-tumor activity against the MC38 tumor in this study, in a dose-dependent manner. Dual combination treatment (Target drug+anti PD-1) produced a superior anti-tumor effect when compared with single agent treatment with either Target drug or anti PD-1, indicating a potential for further clinical evaluation. Treatment with Target drug or Target drug+anti PD-1 not only caused regression of the Target drug injected tumor, but also delayed progression of a distal tumor lesion. Analyzing TILs revealed an increase T cell infiltration in the distal tumor confirming that the tumor regression was mediated by an immune response.

### Example 14. Overall Pharmacology Study Conclusion

The results in the MC38 murine syngeneic tumor model demonstrated that 3-4 intratumoral injections of target drug induced a systemic antitumor effect resulting in regression of not only the injected tumor lesion but also a distal untreated tumor and prolonged the survival of tumor-bearing mice. Analyzing the tumor infiltrating immune cells revealed down-modulation of the myeloid-derived suppressive cells and an enhanced T infiltration. The elicited anti-tumor response was shown to protect the mice against rechallenged MC38 tumor cells. The efficacy of Liposome-packaged RNA replicon expressing IL-12 could be further enhanced when the tumor-bearing animals were simultaneously treated with an anti-mPD-1 antibody.

### Example 15. Efficacy of Target Drug in Tumor Inhibition and Synergy with an Anti-PD-1 Antibody in the B16 Tumor Model in C57BL/6 Mice with Weekly Intravenous Administration

In this study, the therapeutic efficacy of Liposome-packaged RNA replicon (target drug) as a single agent in the treatment of the murine B16 tumor model in C57BL/6 mice by weekly intravenous administration was evaluated. In addition, the intravenous administration of target drug was assessed in combination with a systemic anti-murine PD-1 (anti-mPD-1) antibody in order to re-confirm the effects of a combination treatment relative to the single-agent Target drug treatment. Each treatment group had 16 mice.

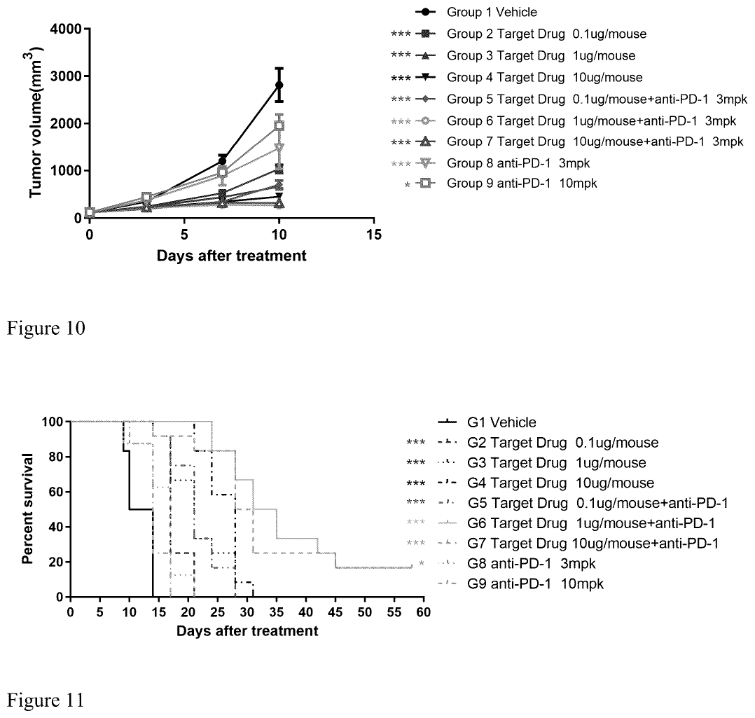

As shown in FIG. 10 and Table 8 below, on Day 10 after starting weekly intravenous administration, treatments with Group 2 (target drug 0.1 ug/mouse), Group 3 (target drug 1 ug/mouse) and Group 4 (target drug 10 ug/mouse) showed significant tumor inhibition in a dose-dependent manner. The Tumor Growth Inhibitions (TGIs) were 63.28%, 76.49% and 84.02%, respectively. All the p value were less than 0.001 compared to Group 1 (vehicle group).

The combination systemic administration of target drug and 3 mpk PD-1 antibody enhanced the target drug's tumor inhibition effect. The TGI were 75.00% (Group 5—target drug 0.1 ug/mouse+3mpk PD-1), 90.41% (Group 6—target drug 1 ug/mouse+3mpk PD-1) and 88.74% (Group 7—target drug 10 ug/mouse+3 mpk PD-1) respectively. PD-1 3mpk and 10mpk monotherapy also showed tumor inhibition effect, with TGIs being 47.59% (Group 8 PD-1 3mpk) and 30.68% (Group 9 PD-1 10mpk), respectively.

TABLE 8
Anti-tumor activity in the different treatment groups
with Weekly IV administration in B16 mouse model

| | | | c - D 10 after | | | | | |
|---|---|---|---|---|---|---|---|---|
| | | | starting | | | | | |
| | | | treatment | | | | | |
| | a | b | | TGI = | | | | |
| | TV | TV | T/C | 1-T/C | Statistical Significance (n = 16) | | | |
| Group | (mm3) | (mm3) | (%) | (%) | d | e | f | g |
| G1 Vehicle | 119 ± 8 | 2816 ± 370 | 100 | 0 | — | **** | ** | |
| G2 Target Drug | 120 ± 5 | 1034 ± 87 | 36.72 | 63.28 | *** | ns | *** | |
| 0.1 ug/mouse | | | | | | | | |
| G3 Target Drug | 120 ± 5 | 662 ± 55 | 23.51 | 76.49 | *** | ** | **** | |
| 1 ug/mouse | | | | | | | | |
| G4 Target Drug | 119 ± 5 | 450 ± 31 | 15.98 | 84.02 | *** | **** | **** | |
| 10 ug/mouse | | | | | | | | |
| G5 Target Drug | 119 ± 4 | 704 ± 86 | 25.00 | 75.00 | *** | ** | **** | * |
| 0.1 ug/mouse + | | | | | | | | |
| anti-PD-1 3 mpk | | | | | | | | |
| G6 Target Drug | 119 ± 4 | 270 ± 37 | 9.59 | 90.41 | *** | **** | **** | **** |
| 1 ug/mouse + | | | | | | | | |
| anti-PD-1 3 mpk | | | | | | | | |
| G7 Target Drug | 120 ± 5 | 317 ± 42 | 11.26 | 88.74 | *** | **** | **** | * |
| 10 ug/mouse + | | | | | | | | |
| anti-PD-1 3 mpk | | | | | | | | |
| G8 anti-PD-1 3 mpk | 123 ± 7 | 1476 ± 444 | 52.41 | 47.59 | *** | — | ns | |
| G9 anti-PD-1 | 121 ± 5 | 1952 ± 239 | 69.32 | 30.68 | * | ns | — | |
| 10 mpk | | | | | | | | |

Note:
a. Tumor volume at Day 0;
b. Tumor volume at Day 10.
c. T/C % = TRTV/CRTV × 100% (RTV = Vt/V0, TGI % = 1-T/C;
d. all groups compare to G1 at Day 10;
e, all groups compare to G8 at Day 10;
f, all groups compare to G9 at Day 10;
g. combination therapy compare to monotherapy dose by dose at Day 10.
*, p < 0.05;
**, p < 0.01,;
***, p < 0.001;
****, p < 0.0001

The survival of tumor-bearing mice is shown in FIG. 11. All mice in the vehicle group were euthanized on Day 14 after starting weekly intravenous treatment, as the tumor size exceeded 2,000 mm³. After 3 times weekly intravenous treatment, the overall survival rates in dose groups of combination group 6 and group 7 were still 20% on Day 58. Three weekly intravenous treatments with target drug alone or in combination with anti-PD1 antibody substantially reduced the tumor growth and prolonged the survival of tumor-bearing mice.

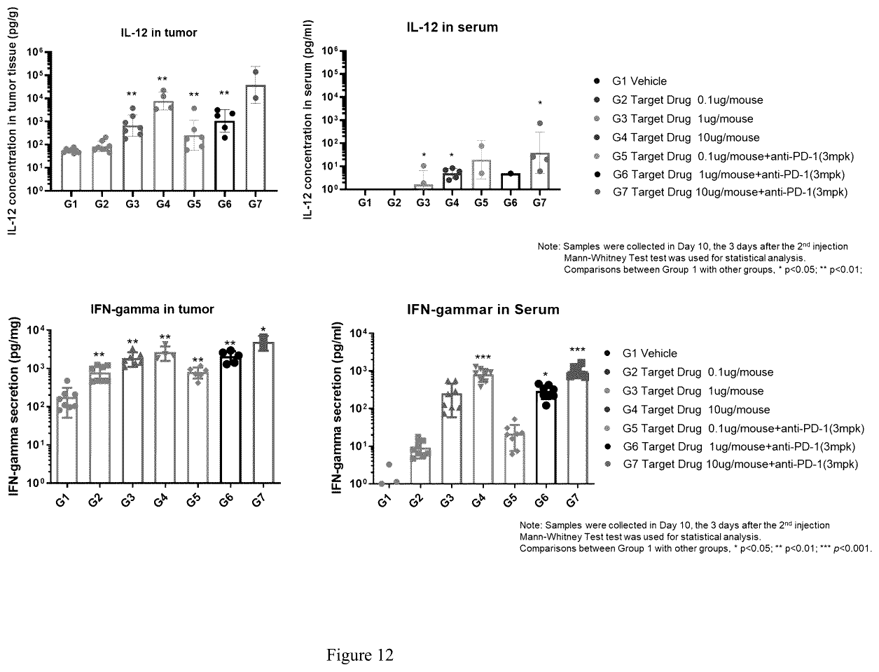

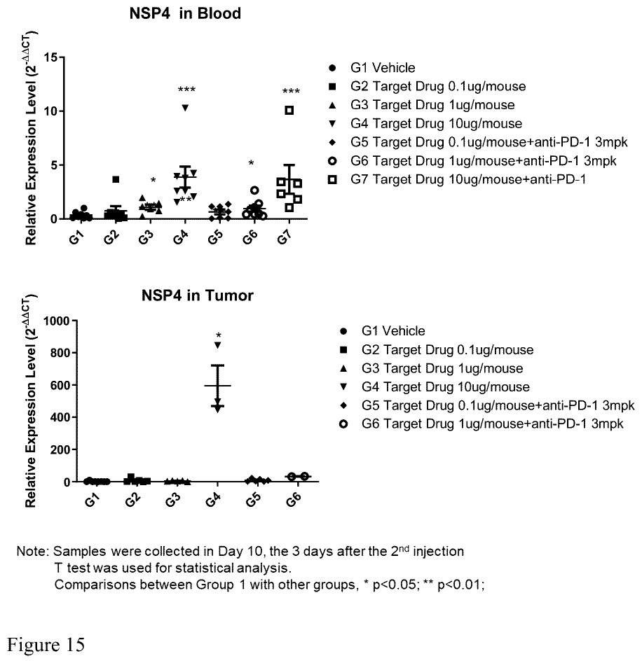

Blood and tumor samples were collected for all groups on Day 10 after starting weekly intravenous administration when some mice in the vehicle group started being euthanized due to tumor size exceeding 2,000 mm³. As shown in FIG. 12, on Day 10 after starting weekly intravenous administration, i.e. Day 3 after second weekly intravenous administration, the changes of cytokines in plasma suggested that the expression of IL-12 increased with IFN-7 and showed an upward trend with the increasing dose for mono or combination treatments. In addition, ELISA assay demonstrated that the expression of IL-12 and IFN-7 in tumor samples also increased after mono or combination treatments (see FIG. 12). As shown in FIG. 15, qPCR tests showed that the expression levels of NSP4 in blood cells and tumor were significantly higher than those of the control group.

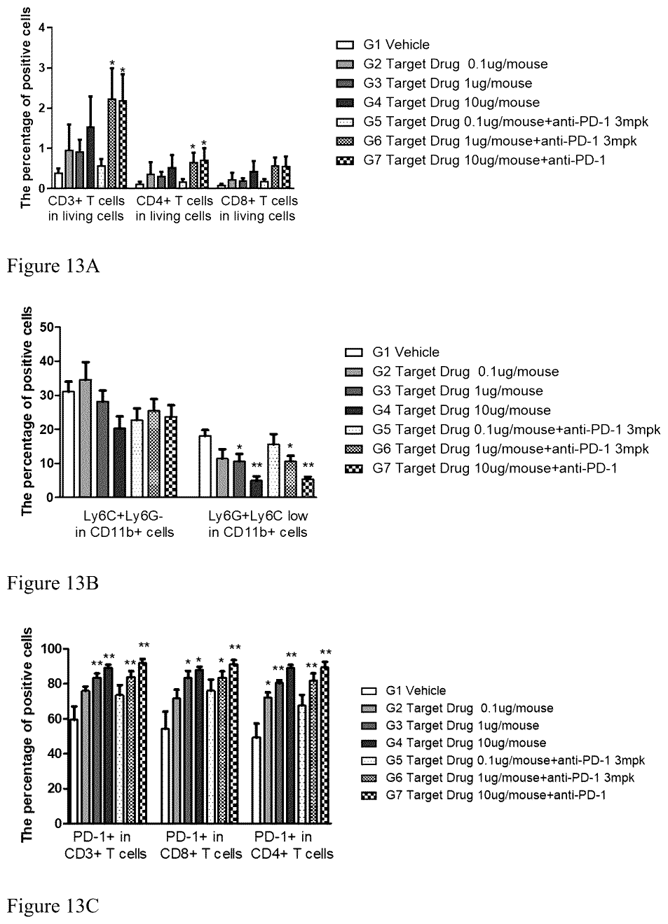

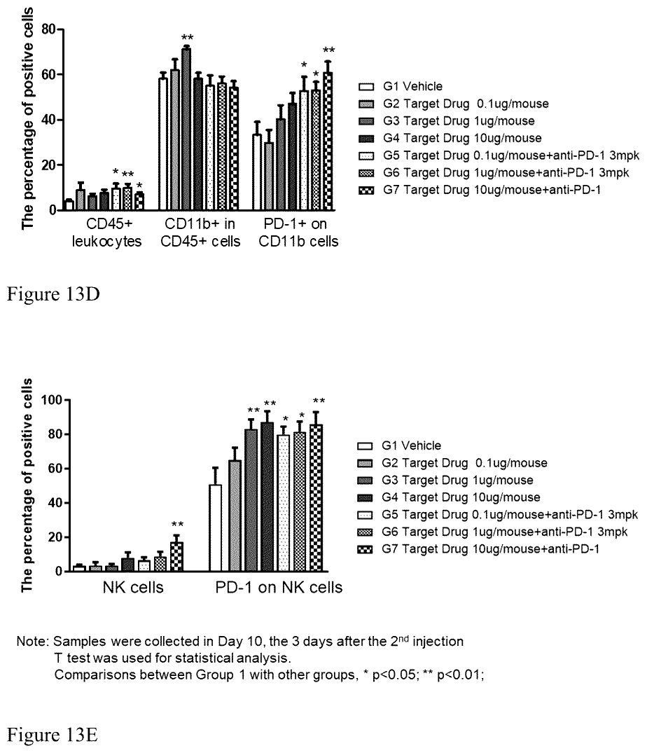

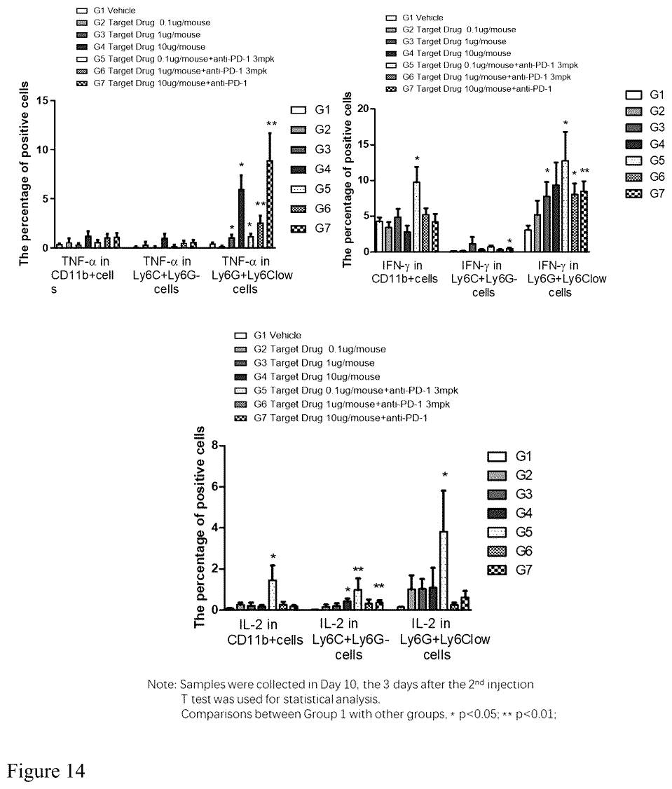

As shown in FIGS. 13A-13E, FACS results showed that the proportion of CD8+ T cells in tumor was increased in some groups injected with the target drug. In addition, the proportion of CD3+ T cells in tumor increased significantly in combination treatment groups 6 & 7. As shown in FIGS. 13A-13E and FIG. 14, the expression of TNF-α, IL-2, IFN-7 (see FIG. 14) and PD-1 (see FIGS. 13A-13E) increased in T cells. As shown in FIGS. 13A-13E, g-MDSCs (Ly6G+Ly6C low) cells of mice given 1 μg and 10 ug target drug were significantly decreased in tumor compared with the control group, while the M-MDSC (Ly6C+Ly6G-) cells showed no significant difference. Also shown in FIGS. 13A-13E, the proportion of NK cells in tumor in Group 7 (target drug 10 ug/mouse+anti-PD-1 3mpl) was significantly increased.

In conclusion, three times weekly systemic administrations of target drug alone had demonstrated a significant therapeutic effect in B16-F10 tumor model in C57BL/6 mice at doses of 0.1 μg/mouse, 1 μg/mouse and 10 ug/mouse and the efficacy were dose-dependent. Three times weekly systemic administration of target drug in combination with anti-PD-1 antibody enhanced target drug's tumor inhibition effect. Target drug expressed IL-12 in mice, and the possible pharmacodynamic mechanisms of the target drug's tumor inhibition effect was mediated by immune response including increased proportion of NK and T cells, decreased proportion of myeloid inhibitory cells, and increased secretion of cytokines such as IFN-7 and TNF-α in tumor tissue.

### Example 16. Efficacy of Target Drug in Tumor Inhibition and Synergy with an Anti-PD-1 Antibody in the B16 Tumor Model in C57BL/6 Mice by Biweekly Intravenous Administration

In this study, the injection cycle of the therapeutic efficacy of Liposome-packaged RNA replicon as a therapeutic agent in the treatment of the murine B16 tumor model in C57BL/6 mice by intravenous administration was extended to biweekly. In addition, the efficacy of the intravenous administration of target drug was assessed in combination with a systemic anti-mPD-1 antibody to re-confirm the effects of a combination treatment relative to the single-agent Target drug treatment. The dosage of anti-mPD-1 administration was changed to 5mpk in this biweekly intravenous administration study. Each treatment group had 8 mice.

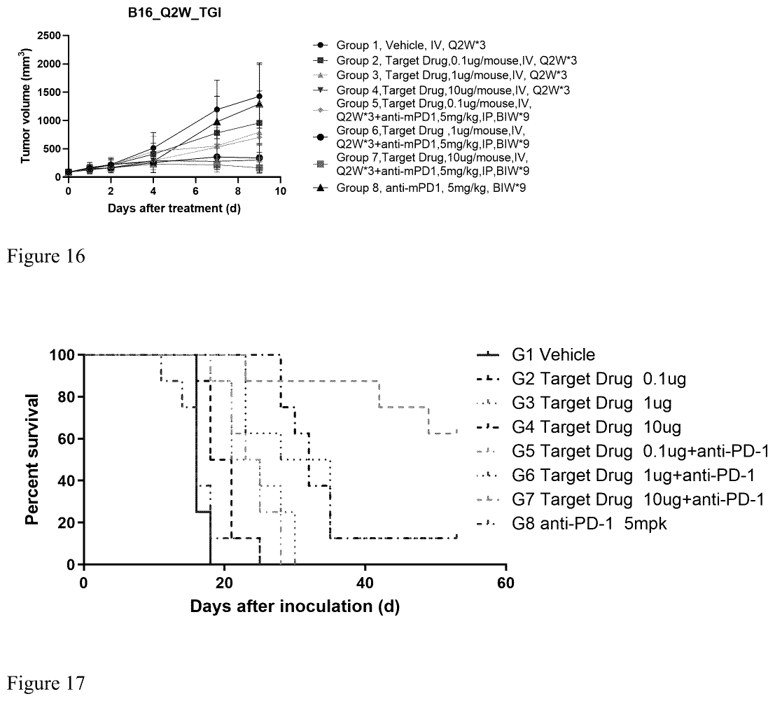

As shown in FIG. 16 and Table 9 below, on Day 9 after target drug weekly intravenous treatment, treatments with Group 2 (target drug 0.1 ug/mouse), Group 3 (target drug 1 ug/mouse) and Group 4 (target drug 10 ug/mouse) showed significant tumor inhibition in a dose-dependent manner. The TGIs were 40.70%, 50.75% and 81.34%, respectively with p value=0.441, 0.1972 and 0.0069 compared to Group 1 (vehicle group).

The combined treatments of target drug and 5mpk PD-1 antibody could enhance the tumor inhibition effect, the TGI were 56.44% (Group 5 target drug 0.1 ug/mouse+5mpk PD-1), 79.03% (Group 6 target drug 1 ug/mouse+5mpk PD-1) and 89.92% (Group 7 target drug 10 ug/mouse+5mpk PD-1) respectively with p value=0.1175, 0.0093 and 0.0022 compared to Group 1 (vehicle group). Group 8 (PD-1 5mpk) monotherapy also showed tumor inhibition effect, with TGIs being 19.92%.

TABLE 9
Anti-tumor activity in the different treatment groups
with Biweekly IV administration in B16 mouse model

| | D 9 after | | | |
|---|---|---|---|---|
| | starting | | | |
| | biweekly iv | Statistical Significance | | |
| | treatment | (n = 8) | | |
| Group | a - TGI (%) ^{a} | b | c | d |
| G1 Vehicle | 0 | — | ns | — |
| G2 Target Drug 0.1 ug/mouse | 40.70 | ns | ns | — |
| G3 Target Drug 1 ug/mouse | 50.75 | ns | ns | — |
| G4 Target Drug 10 ug/mouse | 81.34 | ** | ** | — |
| G5 Target Drug 0.1 ug/mouse + | 56.44 | ns | ns | ns |
| anti-PD-1 5 mpk | | | | |
| G6 Target Drug 1 ug/mouse + | 79.03 | ** | ** | * |
| anti-PD-1 5 mpk | | | | |
| G7 Target Drug 10 ug/mouse + | 89.92 | ** | ** | * |
| anti-PD-1 5 mpk | | | | |
| G8 anti-PD-1 5 mpk | 19.92 | ns | — | — |

Note:
^{a} TGI % = (1 − TRTV/CRTV) × 100% (RTV = Vt/V0);
b. all groups compare to G1 at Day 9;
c, all groups compare to G8 at Day 9;
d. combination therapy compare to monotherapy dose by dose at Day 9,
* p < 0.05;
** p < 0.01,;
*** p < 0.001;
**** p < 0.0001

The probability of survival of B16 tumor-bearing mice with biweekly intravenous treatment is shown in FIG. 17. All mice in the vehicle group were euthanized on Day 16 after tumor inoculation (Day 9 after starting biweekly intravenous treatment), as the tumor size exceeded 2,000 mm³. After 3 times biweekly intravenous treatment, the overall survival rates in group 4 (Target Drug 10 ug/mouse) and group 6 (Target Drug 1 ug/mouse+anti-PD-1 5mpk) were both 12.5% on Day 53 after tumor inoculation (Day 46 after starting biweekly intravenous treatment), while group 7 (Target Drug 1 μg/mouse+anti-PD-1 5mpk) was 62.5%. In conclusion, 3 times biweekly systemic treatment with target drug alone or in combination with anti-PD1 antibody substantially reduced tumor growth and prolonged the survival of B16 tumor-bearing C57BL/6 Mice.

### Example 17. Efficacy of Target Drug in Tumor Inhibition and Synergy with an Anti-PD-1 Antibody in the EMT6 Tumor Model in Balb/c Mice by Biweekly Intravenous Administration

In this study, the therapeutic efficacy of liposome-packaged RNA replicon encoding IL-12 as a therapeutic agent in the treatment of the murine EMT6 tumor model in Balb/c mice with biweekly intravenous administration was evaluated. Furthermore, the biweekly intravenous administration of target drug was assessed in combination with a systemic anti-mPD-1 antibody to re-confirm the synergetic effects. The dosage of anti-mPD-1 administration was 5mpk. Each treatment group had 8 mice.

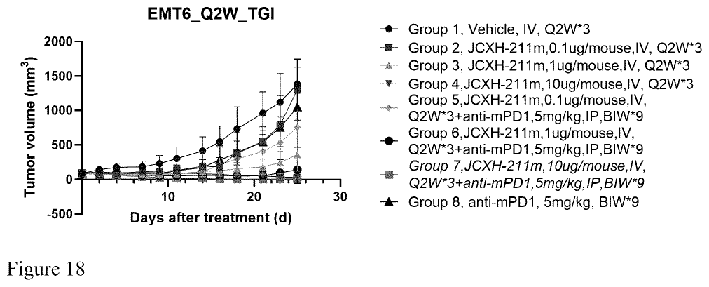

As shown in FIG. 18 and Table 10 below, on 25 days after treatment, single treatment with Target drug at dose level of 0.1 μg/mouse (Group 2) produced a weak antitumor activity and the TGI was 5.8% with p=0.9974 compared with the vehicle group (Group 1). Single treatment with the target drug at dose level of 1 μg/mouse (Group 3) and 10 μg/mouse (Group 4) produced remarkable anti-tumor activity and the TGI were 74.03% and 97.93%, respectively with p value <0.0001 compared to the vehicle group (Group 1).

Combination treatment of Target drug at dose level of 0.1, 1, and 10 μg/mouse with anti-mPD-1 at dose level of 5 mg/kg (Groups 5-7) produced tremendous anti-tumor activities. The TGI were 45.28%, 90.02% and 99.32% respectively with p=0.0143, <0.0001 and <0.0001 compared to the vehicle group (Group 1).

TABLE 10
Anti-tumor activity in the different treatment groups with
biweekly IV administration in EMT6 mouse tumor model

| | D 9 after | | | |
|---|---|---|---|---|
| | starting | | | |
| | biweekly iv | Statistical Significance | | |
| | treatment | (n = 8) | | |
| Group | a - TGI (%) | b | c | d |
| G1 Vehicle | 0 | — | ns | — |
| G2 Target Drug 0.1 ug/mouse | 5.8 | ns | ns | — |
| G3 Target Drug 1 ug/mouse | 74.03 | **** | ** | — |
| G4 Target Drug 10 ug/mouse | 97.93 | **** | **** | — |
| G5 Target Drug 0.1 ug/mouse + | 45.28 | * | ns | * |
| anti-PD-1 5 mpk | | | | |
| G6 Target Drug 1 ug/mouse + | 90.02 | **** | **** | ns |
| anti-PD-1 5 mpk | | | | |
| G7 Target Drug 10 ug/mouse + | 99.32 | **** | **** | ns |
| anti-PD-1 5 mpk | | | | |
| G8 anti-PD-1 5 mpk | 19.92 | ns | — | — |

Note:
a. TGI % = (1 − TRTV/CRTV) × 100% (RTV = Vt/V0);
b. all groups compare to G1 at Day 9;
c, all groups compare to G8 at Day 9;
d. combination therapy compare to monotherapy dose by dose at Day 9,
* p < 0.05;
** p < 0.01,;
*** p < 0.001;
**** p < 0.0001

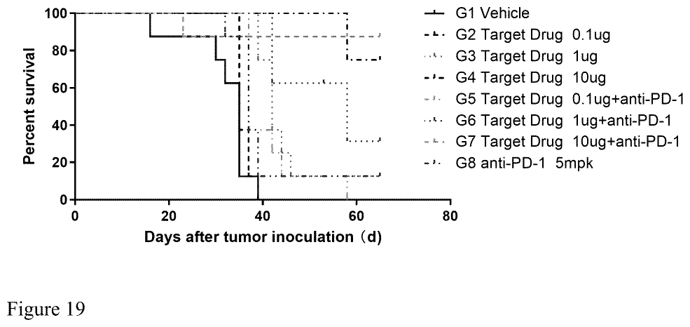

The survival of tumor-bearing mice is shown in FIG. 19. All mice in the vehicle group were euthanized on Day 32 after tumor inoculation (Day 25 after starting biweekly intravenous treatment), as the tumor size exceeded 2,000 mm3. After 3 times biweekly intravenous treatment, on Day 65 after tumor inoculation (Day 58 after starting biweekly intravenous treatment), the overall survival rates in group 3 (Target Drug 1 μg/mouse), group 4 Target Drug 10 ug/mouse), group 6 (Target Drug 1 μg/mouse+anti-PD-1 5mpk) and group 7 (Target Drug 10 ug/mouse+anti-PD-1 5mpk) were 12.5%, 75%, 37.5% and 87.5%, respectively. In conclusion, 3 times biweekly intravenous treatment with target drug alone or in combination with anti-PD1 antibody substantially reduced the tumor growth and prolonged the survival of EMT6 tumor-bearing mice.

### Example 18. Overall Pharmacology Study Conclusion

The results in both of the B16 and EMT6 murine syngeneic tumor model demonstrated that 3 intravenous injections of target drug induced a systemic antitumor effect resulting in regression and prolonged the survival of tumor-bearing mice. Analyzing the tumor infiltrating immune cells revealed down-modulation of the myeloid-derived suppressive cells and an enhanced T cell and NK cell infiltration, and increased secretion of cytokines such as IFN-γ and TNF-α, the same as in the MC38 tumor model with intratumorally injection as shown in examples 10-14. The efficacy of Liposome-packaged RNA replicon expressing IL-12 could also be further enhanced when the tumor-bearing animals were simultaneously treated with an anti-mPD-1 antibody.

### INCORPORATION BY REFERENCE

The entire disclosure of each of the patent documents and scientific articles cited herein is incorporated by reference for all purposes.

### SEQUENCE LISTING

| | |
|---|---|
| SEQ ID NO: 1 | AUGUGCCACCAGCAGCUGGUCAUCAGCUGGUUCAGCCUCGUUUUCC |
| IL-12 (p40, linker, | UCGCCUCGCCGCUGGUCGCCAUAUGGGAGCUCAAGAAGGACGUAUA |
| p35) | CGUGGUGGAGCUGGACUGGUACCCCGACGCGCCGGGCGAGAUGGUC |
| | GUCCUGACGUGCGACACGCCGGAGGAGGACGGCAUCACGUGGACGC |
| | UGGACCAGUCCAGCGAGGUCCUCGGCUCCGGCAAGACGCUGACGAU |
| | CCAGGUCAAGGAGUUCGGCGACGCGGGCCAGUACACGUGCCACAAG |
| | GGCGGCGAGGUCCUGAGCCACUCCCUCCUCCUGCUACACAAGAAGG |
| | AGGACGGGAUCUGGAGCACGGACAUCCUCAAGGACCAGAAGGAGC |
| | CGAAGAACAAGACCUUCCUGCGCUGCGAGGCGAAGAAUUACUCGG |
| | GCCGGUUCACGUGCUGGUGGCUCACCACGAUCAGCACGGACCUGAC |
| | GUUCUCGGUCAAGUCGUCGCGGGGCUCGUCGGACCCCCAGGGGGUG |
| | ACCUGCGGCGCGGCGACGCUGUCGGCGGAGCGGGUGCGGGGCGACA |
| | ACAAGGAGUACGAGUACUCGGUCGAGUGCCAGGAGGACUCGGCGU |
| | GCCCGGCGGCGGAGGAGUCGCUGCCGAUCGAGGUGAUGGUCGACGC |
| | GGUCCACAAGCUGAAGUACGAGAACUACACGUCGUCGUUCUUCAUC |
| | CGGGACAUCAUCAAGCCGGACCCGCCGAAGAACCUGCAGCUGAAGC |
| | CGCUGAAGAACUCGCGGCAGGUCGAGGUCUCGUGGGAGUACCCGG |
| | ACACGUGGUCGACGCCGCACUCGUACUUCUCGCUGACGUUCUGCGU |
| | CCAAGUGCAGGGCAAGUCGAAGCGGGAGAAGAAGGACCGGGUGUU |
| | CACCGACAAGACGAGCGCGACGGUGAUCUGCCGGAAGAACGCGUCG |
| | AUCUCGGUGCGGGCGCAGGACCGGUACUACUCGUCGUCGUGGUCGG |
| | AGUGGGCGUCGGUGCCGUGCAGCGGCGGAAGCGGCGGCGGCAGCG |
| | GCGGAGGCUCUGGCGGCGGCUCCCGGAACCUGCCGGUGGCGACGCC |
| | GGACCCGGGGAUGUUCCCGUGCCUGCACCACAGCCAGAACCUGCUG |
| | CGGGCGGUGUCGAACAUGCUGCAGAAGGCGCGGCAGACGCUGGAG |
| | UUCUACCCGUGCACGAGCGAGGAGAUCGACCACGAGGACAUCACGA |
| | AGGACAAGACCAGCACGGUGGAGGCGUGCCUGCCGCUGGAGCUGAC |
| | GAAGAACGAGUCGUGCCUGAACUCGAGGGAGACGUCGUUCAUCAC |
| | GAACGGGUCGUGCCUGGCGUCGCGGAAGACGUCGUUCAUGAUGGC |
| | GCUGUGCCUGUCGUCGAUCUACGAGGACCUGAAGAUGUACCAGGU |
| | GGAGUUCAAGACGAUGAACGCGAAGCUGCUGAUGGACCCGAAGCG |
| | GCAGAUCUUCCUCGACCAGAACAUGCUGGCGGUGAUCGACGAGCUC |
| | AUGCAGGCGCUCAACUUCAACAGCGAGACGGUGCCGCAGAAGUCGU |
| | CGCUCGAGGAGCCGGACUUCUACAAGACGAAGAUCAAGCUCUGCAU |
| | CCUGCUGCACGCUUUCCGGAUCCGGGCGGUGACGAUCGACCGGGUG |
| | AUGUCGUACCUGAACGCUUCGUGAUAA |
| SEQ ID NO: 2 | AUGUGCCACCAGCAGCUGGUCAUCAGCUGGUUCAGCCUCGUUUUCC |
| Subunit p40 | UCGCCUCGCCGCUGGUCGCCAUAUGGGAGCUCAAGAAGGACGUAUA |
| | CGUGGUGGAGCUGGACUGGUACCCCGACGCGCCGGGCGAGAUGGUC |
| | GUCCUGACGUGCGACACGCCGGAGGAGGACGGCAUCACGUGGACGC |
| | UGGACCAGUCCAGCGAGGUCCUCGGCUCCGGCAAGACGCUGACGAU |
| | CCAGGUCAAGGAGUUCGGCGACGCGGGCCAGUACACGUGCCACAAG |
| | GGCGGCGAGGUCCUGAGCCACUCCCUCCUCCUGCUACACAAGAAGG |
| | AGGACGGGAUCUGGAGCACGGACAUCCUCAAGGACCAGAAGGAGC |
| | CGAAGAACAAGACCUUCCUGCGCUGCGAGGCGAAGAAUUACUCGG |
| | GCCGGUUCACGUGCUGGUGGCUCACCACGAUCAGCACGGACCUGAC |
| | GUUCUCGGUCAAGUCGUCGCGGGGCUCGUCGGACCCCCAGGGGGUG |
| | ACCUGCGGCGCGGCGACGCUGUCGGCGGAGCGGGUGCGGGGCGACA |
| | ACAAGGAGUACGAGUACUCGGUCGAGUGCCAGGAGGACUCGGCGU |
| | GCCCGGCGGCGGAGGAGUCGCUGCCGAUCGAGGUGAUGGUCGACGC |
| | GGUCCACAAGCUGAAGUACGAGAACUACACGUCGUCGUUCUUCAUC |
| | CGGGACAUCAUCAAGCCGGACCCGCCGAAGAACCUGCAGCUGAAGC |
| | CGCUGAAGAACUCGCGGCAGGUCGAGGUCUCGUGGGAGUACCCGG |
| | ACACGUGGUCGACGCCGCACUCGUACUUCUCGCUGACGUUCUGCGU |
| | CCAAGUGCAGGGCAAGUCGAAGCGGGAGAAGAAGGACCGGGUGUU |
| | CACCGACAAGACGAGCGCGACGGUGAUCUGCCGGAAGAACGCGUCG |
| | AUCUCGGUGCGGGCGCAGGACCGGUACUACUCGUCGUCGUGGUCGG |
| | AGUGGGCGUCGGUGCCGUGCAGCGGC |
| SEQ ID NO: 3 | CGGAACCUGCCGGUGGCGACGCCGGACCCGGGGAUGUUCCCGUGCC |
| Subunit p35 | UGCACCACAGCCAGAACCUGCUGCGGGCGGUGUCGAACAUGCUGCA |
| | GAAGGCGCGGCAGACGCUGGAGUUCUACCCGUGCACGAGCGAGGA |
| | GAUCGACCACGAGGACAUCACGAAGGACAAGACCAGCACGGUGGA |
| | GGCGUGCCUGCCGCUGGAGCUGACGAAGAACGAGUCGUGCCUGAAC |
| | UCGAGGGAGACGUCGUUCAUCACGAACGGGUCGUGCCUGGCGUCGC |
| | GGAAGACGUCGUUCAUGAUGGCGCUGUGCCUGUCGUCGAUCUACG |
| | AGGACCUGAAGAUGUACCAGGUGGAGUUCAAGACGAUGAACGCGA |
| | AGCUGCUGAUGGACCCGAAGCGGCAGAUCUUCCUCGACCAGAACAU |
| | GCUGGCGGUGAUCGACGAGCUCAUGCAGGCGCUCAACUUCAACAGC |
| | GAGACGGUGCCGCAGAAGUCGUCGCUCGAGGAGCCGGACUUCUACA |
| | AGACGAAGAUCAAGCUCUGCAUCCUGCUGCACGCUUUCCGGAUCCG |
| | GGCGGUGACGAUCGACCGGGUGAUGUCGUACCUGAACGCUUCGUG |
| | AUAA |
| SEQ ID NO: 4 | GGAAGCGGCGGCGGCAGCGGCGGAGGCUCUGGCGGCGGCUCC |
| linker | |
| SEQ ID NO: 5 | AUGGAGAAAGUUCACGUUGACAUCGAGGAAGACAGCCCAUUCCUC |
| srRNA sequence | AGAGCUUUGCAGCGGAGCUUCCCGCAGUUUGAGGUAGAAGCCAAG |
| | CAGGUCACUGAUAAUGACCAUGCUAAUGCCAGAGCGUUUUCGCAU |
| | CUGGCUUCAAAACUGAUCGAAACGGAGGUGGACCCAUCCGACACGA |
| | UCCUUGACAUUGGAAGUGCGCCCGCCCGCAGAAUGUAUUCUAAGCA |
| | CAAGUAUCAUUGUAUCUGUCCGAUGAGAUGUGCGGAAGAUCCGGA |
| | CAGAUUGUAUAAGUAUGCAACUAAGCUGAAGAAAAACUGUAAGGA |
| | AAUAACUGAUAAGGAAUUGGACAAGAAAAUGAAGGAGCUCGCCGC |
| | CGUCAUGAGCGACCCUGACCUGGAAACUGAGACUAUGUGCCUCCAC |
| | GACGACGAGUCGUGUCGCUACGAAGGGCAAGUCGCUGUUUACCAG |
| | GAUGUAUACGCGGUUGACGGACCGACAAGUCUCUAUCACCAAGCCA |
| | AUAAGGGAGUUAGAGUCGCCUACUGGAUAGGCUUUGACACCACCC |
| | CUUUUAUGUUUAAGAACUUGGCUGGAGCAUAUCCAUCAUACUCUA |
| | CCAACUGGGCCGACGAAACCGUGUUAACGGCUCGUAACAUAGGCCU |
| | AUGCAGCUCUGACGUUAUGGAGCGGUCACGUAGAGGGAUGUCCAU |
| | UCUUAGAAAGAAGUAUUUGAAACCAUCCAACAAUGUUCUAUUCUC |
| | UGUUGGCUCGACCAUCUACCACGAGAAGAGGGACUUACUGAGGAG |
| | CUGGCACCUGCCGUCUGUAUUUCACUUACGUGGCAAGCAAAAUUAC |
| | ACAUGUCGGUGUGAGACUAUAGUUAGUUGCGACGGGUACGUCGUU |
| | AAAAGAAUAGCUAUCAGUCCAGGCCUGUAUGGGAAGCCUUCAGGC |
| | UAUGCUGCUACGAUGCACCGCGAGGGAUUCUUGUGCUGCAAAGUG |
| | ACAGACACAUUGAACGGGGAGAGGGUCUCUUUUCCCGUGUGCACG |
| | UAUGUGCCAGCUACAUUGUGUGACCAAAUGACUGGCAUACUGGCA |
| | ACAGAUGUCAGUGCGGACGACGCGCAAAAACUGCUGGUUGGGCUC |
| | AACCAGCGUAUAGUCGUCAACGGUCGCACCCAGAGAAACACCAAUA |
| | CCAUGAAAAAUUACCUUUUGCCCGUAGUGGCCCAGGCAUUUGCUA |
| | GGUGGGCAAAGGAAUAUAAGGAAGAUCAAGAAGAUGAAAGGCCAC |
| | UAGGACUACGAGAUAGACAGUUAGUCAUGGGGUGUUGUUGGGCUU |
| | UUAGAAGGCACAAGAUAACAUCUAUUUAUAAGCGCCCGGAUACCC |
| | AAACCAUCAUCAAAGUGAACAGCGAUUUCCACUCAUUCGUGCUGCC |
| | CAGGAUAGGCAGUAACACAUUGGAGAUCGGGCUGAGAACAAGAAU |
| | CAGGAAAAUGUUAGAGGAGCACAAGGAGCCGUCACCUCUCAUUAC |
| | CGCCGAGGACGUACAAGAAGCUAAGUGCGCAGCCGAUGAGGCUAA |
| | GGAGGUGCGUGAAGCCGAGGAGUUGCGCGCAGCUCUACCACCUUU |
| | GGCAGCUGAUGUUGAGGAGCCCACUCUGGAAGCCGAUGUCGACUU |
| | GAUGUUACAAGAGGCUGGGGCCGGCUCAGUGGAGACACCUCGUGG |
| | CUUGAUAAAGGUUACCAGCUACGAUGGCGAGGACAAGAUCGGCUC |
| | UUACGCUGUGCUUUCUCCGCAGGCUGUACUCAAGAGUGAAAAAUU |
| | AUCUUGCAUCCACCCUCUCGCUGAACAAGUCAUAGUGAUAACACAC |
| | UCUGGCCGAAAAGGGCGUUAUGCCGUGGAACCAUACCAUGGUAAA |
| | GUAGUGGUGCCAGAGGGACAUGCAAUACCCGUCCAGGACUUUCAA |
| | GCUCUGAGUGAAAGUGCCACCAUUGUGUACAACGAACGUGAGUUC |
| | GUAAACAGGUACCUGCACCAUAUUGCCACACAUGGAGGAGCGCUG |
| | AACACUGAUGAAGAAUAUUACAAAACUGUCAAGCCCAGCGAGCAC |
| | GACGGCGAAUACCUGUACGACAUCGACAGGAAACAGUGCGUCAAG |
| | AAAGAACUAGUCACUGGGCUAGGGCUCACAGGCGAGCUGGUGGAU |
| | CCUCCCUUCCAUGAAUUCGCCUACGAGAGUCUGAGAACACGACCAG |
| | CCGCUCCUUACCAAGUACCAACCAUAGGGGUGUAUGGCGUGCCAGG |
| | AUCAGGCAAGUCUGGCAUCAUUAAAAGCGCAGUCACCAAAAAAGA |
| | UCUAGUGGUGAGCGCCAAGAAAGAAAACUGUGCAGAAAUUAUAAG |
| | GGACGUCAAGAAAAUGAAAGGGCUGGACGUCAAUGCCAGAACUGU |
| | GGACUCAGUGCUCUUGAAUGGAUGCAAACACCCCGUAGAGACCCUG |
| | UAUAUUGACGAAGCUUUUGCUUGUCAUGCAGGUACUCUCAGAGCG |
| | CUCAUAGCCAUUAUAAGACCUAAAAAGGCAGUGCUCUGCGGGGAU |
| | CCCAAACAGUGCGGUUUUUUUAACAUGAUGUGCCUGAAAGUGCAU |
| | UUUAACCACGAGAUUUGCACACAAGUCUUCCACAAAAGCAUCUCUC |
| | GCCGUUGCACUAAAUCUGUGACUUCGGUCGUCUCAACCUUGUUUU |
| | ACGACAAAAAAAUGAGAACGACGAAUCCGAAAGAGACUAAGAUUG |
| | UGAUUGACACUACCGGCAGUACCAAACCUAAGCAGGACGAUCUCAU |
| | UCUCACUUGUUUCAGAGGGUGGGUGAAGCAGUUGCAAAUAGAUUA |
| | CAAAGGCAACGAAAUAAUGACGGCAGCUGCCUCUCAAGGGCUGACC |
| | CGUAAAGGUGUGUAUGCCGUUCGGUACAAGGUGAAUGAAAAUCCU |
| | CUGUACGCACCCACCUCAGAACAUGUGAACGUCCUACUGACCCGCA |
| | CGGAGGACCGCAUCGUGUGGAAAACACUAGCCGGCGACCCAUGGAU |
| | AAAAACACUGACUGCCAAGUACCCUGGGAAUUUCACUGCCACGAUA |
| | GAGGAGUGGCAAGCAGAGCAUGAUGCCAUCAUGAGGCACAUCUUG |
| | GAGAGACCGGACCCUACCGACGUCUUCCAGAAUAAGGCAAACGUGU |
| | GUUGGGCCAAGGCUUUAGUGCCGGUGCUGAAGACCGCUGGCAUAG |
| | ACAUGACCACUGAACAAUGGAACACUGUGGAUUAUUUUGAAACGG |
| | ACAAAGCUCACUCAGCAGAGAUAGUAUUGAACCAACUAUGCGUGA |
| | GGUUCUUUGGACUCGAUCUGGACUCCGGUCUAUUUUCUGCACCCAC |
| | UGUUCCGUUAUCCAUUAGGAAUAAUCACUGGGAUAACUCCCCGUC |
| | GCCUAACAUGUACGGGCUGAAUAAAGAAGUGGUCCGUCAGCUCUC |
| | UCGCAGGUACCCACAACUGCCUCGGGCAGUUGCCACUGGAAGAGUC |
| | UAUGACAUGAACACUGGUACACUGCGCAAUUAUGAUCCGCGCAUA |
| | AACCUAGUACCUGUAAACAGAAGACUGCCUCAUGCUUUAGUCCUCC |
| | ACCAUAAUGAACACCCACAGAGUGACUUUUCUUCAUUCGUCAGCAA |
| | AUUGAAGGGCAGAACUGUCCUGGUGGUCGGGGAAAAGUUGUCCGU |
| | CCCAGGCAAAAUGGUUGACUGGUUGUCAGACCGGCCUGAGGCUACC |
| | UUCAGAGCUCGGCUGGAUUUAGGCAUCCCAGGUGAUGUGCCCAAA |
| | UAUGACAUAAUAUUUGUUAAUGUGAGGACCCCAUAUAAAUACCAU |
| | CACUAUCAGCAGUGUGAAGACCAUGCCAUUAAGCUUAGCAUGUUG |
| | ACCAAGAAAGCUUGUCUGCAUCUGAAUCCCGGCGGAACCUGUGUCA |
| | GCAUAGGUUAUGGUUACGCUGACAGGGCCAGCGAAAGCAUCAUUG |
| | GUGCUAUAGCGCGGCAGUUCAAGUUUUCCCGGGUAUGCAAACCGA |
| | AAUCCUCACUUGAAGAGACGGAAGUUCUGUUUGUAUUCAUUGGGU |
| | ACGAUCGCAAGGCCCGUACGCACAAUCCUUACAAGCUUUCAUCAAC |
| | CUUGACCAACAUUUAUACAGGUUCCAGACUCCACGAAGCCGGAUGU |
| | GCACCCUCAUAUCAUGUGGUGCGAGGGGAUAUUGCCACGGCCACCG |
| | AAGGAGUGAUUAUAAAUGCUGCUAACAGCAAAGGACAACCUGGCG |
| | GAGGGGUGUGCGGAGCGCUGUAUAAGAAAUUCCCGGAAAGCUUCG |
| | AUUUACAGCCGAUCGAAGUAGGAAAAGCGCGACUGGUCAAAGGUG |
| | CAGCUAAACAUAUCAUUCAUGCCGUAGGACCAAACUUCAACAAAG |
| | UUUCGGAGGUUGAAGGUGACAAACAGUUGGCAGAGGCUUAUGAGU |
| | CCAUCGCUAAGAUUGUCAACGAUAACAAUUACAAGUCAGUAGCGA |
| | UUCCACUGUUGUCCACCGGCAUCUUUUCCGGGAACAAAGAUCGACU |
| | AACCCAAUCAUUGAACCAUUUGCUGACAGCUUUAGACACCACUGAU |
| | GCAGAUGUAGCCAUAUACUGCAGGGACAAGAAAUGGGAAAUGACU |
| | CUCAAGGAAGCAGUGGCUAGGAGAGAAGCAGUGGAGGAGAUAUGC |
| | AUAUCCGACGACUCUUCAGUGACAGAACCUGAUGCAGAGCUGGUG |
| | AGGGUGCAUCCGAAGAGUUCUUUGGCUGGAAGGAAGGGCUACAGC |
| | ACAAGCGAUGGCAAAACUUUCUCAUAUUUGGAAGGGACCAAGUUU |
| | CACCAGGCGGCCAAGGAUAUAGCAGAAAUUAAUGCCAUGUGGCCC |
| | GUUGCAACGGAGGCCAAUGAGCAGGUAUGCAUGUAUAUCCUCGGA |
| | GAAAGCAUGAGCAGUAUUAGGUCGAAAUGCCCCGUCGAAGAGUCG |
| | GAAGCCUCCACACCACCUAGCACGCUGCCUUGCUUGUGCAUCCAUG |
| | CCAUGACUCCAGAAAGAGUACAGCGCCUAAAAGCCUCACGUCCAGA |
| | ACAAAUUACUGUGUGCUCAUCCUUUCCAUUGCCGAAGUAUAGAAU |
| | CACUGGUGUGCAGAAGAUCCAAUGCUCCCAGCCUAUAUUGUUCUCA |
| | CCGAAAGUGCCUGCGUAUAUUCAUCCAAGGAAGUAUCUCGUGGAA |
| | ACACCACCGGUAGACGAGACUCCGGAGCCAUCGGCAGAGAACCAAU |
| | CCACAGAGGGGACACCUGAACAACCACCACUUAUAACCGAGGAUGA |
| | GACCAGGACUAGAACGCCUGAGCCGAUCAUCAUCGAAGAGGAAGA |
| | AGAGGAUAGCAUAAGUUUGCUGUCAGAUGGCCCGACCCACCAGGU |
| | GCUGCAAGUCGAGGCAGACAUUCACGGGCCGCCCUCUGUAUCUAGC |
| | UCAUCCUGGUCCAUUCCUCAUGCAUCCGACUUUGAUGUGGACAGUU |
| | UAUCCAUACUUGACACCCUGGAGGGAGCUAGCGUGACCAGCGGGGC |
| | AACGUCAGCCGAGACUAACUCUUACUUCGCAAAGAGUAUGGAGUU |
| | UCUGGCGCGACCGGUGCCUGCGCCUCGAACAGUAUUCAGGAACCCU |
| | CCACAUCCCGCUCCGCGCACAAGAACACCGUCACUUGCACCCAGCA |
| | GGGCCUGCUCGAGAACCAGCCUAGUUUCCACCCCGCCAGGCGUGAA |
| | UAGGGUGAUCACUAGAGAGGAGCUCGAGGCGCUUACCCCGUCACGC |
| | ACUCCUAGCAGGUCGGUCUCGAGAACCAGCCUGGUCUCCAACCCGC |
| | CAGGCGUAAAUAGGGUGAUUACAAGAGAGGAGUUUGAGGCGUUCG |
| | UAGCACAACAACAAUGACGGUUUGAUGCGGGUGCAUACAUCUUUU |
| | CCUCCGACACCGGUCAAGGGCAUUUACAACAAAAAUCAGUAAGGCA |
| | AACGGUGCUAUCCGAAGUGGUGUUGGAGAGGACCGAAUUGGAGAU |
| | UUCGUAUGCCCCGCGCCUCGACCAAGAAAAAGAAGAAUUACUACGC |
| | AAGAAAUUACAGUUAAAUCCCACACCUGCUAACAGAAGCAGAUAC |
| | CAGUCCAGGAAGGUGGAGAACAUGAAAGCCAUAACAGCUAGACGU |
| | AUUCUGCAAGGCCUAGGGCAUUAUUUGAAGGCAGAAGGAAAAGUG |
| | GAGUGCUACCGAACCCUGCAUCCUGUUCCUUUGUAUUCAUCUAGUG |
| | UGAACCGUGCCUUUUCAAGCCCCAAGGUCGCAGUGGAAGCCUGUAA |
| | CGCCAUGUUGAAAGAGAACUUUCCGACUGUGGCUUCUUACUGUAU |
| | UAUUCCAGAGUACGAUGCCUAUUUGGACAUGGUUGACGGAGCUUC |
| | AUGCUGCUUAGACACUGCCAGUUUUUGCCCUGCAAAGCUGCGCAGC |
| | UUUCCAAAGAAACACUCCUAUUUGGAACCCACAAUACGAUCGGCAG |
| | UGCCUUCAGCGAUCCAGAACACGCUCCAGAACGUCCUGGCAGCUGC |
| | CACAAAAAGAAAUUGCAAUGUCACGCAAAUGAGAGAAUUGCCCGU |
| | AUUGGAUUCGGCGGCCUUUAAUGUGGAAUGCUUCAAGAAAUAUGC |
| | GUGUAAUAAUGAAUAUUGGGAAACGUUUAAAGAAAACCCCAUCAG |
| | GCUUACUGAAGAAAACGUGGUAAAUUACAUUACCAAAUUAAAAGG |
| | ACCAAAAGCUGCUGCUCUUUUUGCGAAGACACAUAAUUUGAAUAU |
| | GUUGCAGGACAUACCAAUGGACAGGUUUGUAAUGGACUUAAAGAG |
| | AGACGUGAAAGUGACUCCAGGAACAAAACAUACUGAAGAACGGCC |
| | CAAGGUACAGGUGAUCCAGGCUGCCGAUCCGCUAGCAACAGCGUAU |
| | CUGUGCGGAAUCCACCGAGAGCUGGUUAGGAGAUUAAAUGCGGUC |
| | CUGCUUCCGAACAUUCAUACACUGUUUGAUAUGUCGGCUGAAGAC |
| | UUUGACGCUAUUAUAGCCGAGCACUUCCAGCCUGGGGAUUGUGUU |
| | CUGGAAACUGACAUCGCGUCGUUUGAUAAAAGUGAGGACGACGCC |
| | AUGGCUCUGACCGCGUUAAUGAUUCUGGAAGACUUAGGUGUGGAC |
| | GCAGAGCUGUUGACGCUGAUUGAGGCGGCUUUCGGCGAAAUUUCA |
| | UCAAUACAUUUGCCCACUAAAACUAAAUUUAAAUUCGGAGCCAUG |
| | AUGAAAUCUGGAAUGUUCCUCACACUGUUUGUGAACACAGUCAUU |
| | AACAUUGUAAUCGCAAGCAGAGUGUUGAGAGAACGGCUAACCGGA |
| | UCACCAUGUGCAGCAUUCAUUGGAGAUGACAAUAUCGUGAAAGGA |
| | GUCAAAUCGGACAAAUUAAUGGCAGACAGGUGCGCCACCUGGUUG |
| | AAUAUGGAAGUCAAGAUUAUAGAUGCUGUGGUGGGCGAGAAAGCG |
| | CCUUAUUUCUGUGGAGGGUUUAUUUUGUGUGACUCCGUGACCGGC |
| | ACAGCGUGCCGUGUGGCAGACCCCCUAAAAAGGCUGUUUAAGCUU |
| | GGCAAACCUCUGGCAGCAGACGAUGAACAUGAUGAUGACAGGAGA |
| | AGGGCAUUGCAUGAAGAGUCAACACGCUGGAACCGAGUGGGUAUU |
| | CUUUCAGAGCUGUGCAAGGCAGUAGAAUCAAGGUAUGAAACCGUA |
| | GGAACUUCCAUCAUAGUUAUGGCCAUGACUACUCUAGCUAGCAGU |
| | GUUAAAUCAUUCAGCUACCUGAGAGGGGCC |
| SEQ ID NO: 6 | MCHQQLVISWFSLVFLASPLVAIWELKKDVYVVELDWYPDAPGEMVVL |
| IL-12 sequence | TCDTPEEDGITWTLDQSSEVLGSGKTLTIQVKEFGDAGQYTCHKGGEVL |
| | SHSLLLLHKKEDGIWSTDILKDQKEPKNKTFLRCEAKNYSGRFTCWWLT |
| | TISTDLTFSVKSSRGSSDPQGVTCGAATLSAERVRGDNKEYEYSVECQED |
| | SACPAAEESLPIEVMVDAVHKLKYENYTSSFFIRDIIKPDPPKNLQLKPLK |
| | NSRQVEVSWEYPDTWSTPHSYFSLTFCVQVQGKSKREKKDRVFTDKTS |
| | ATVICRKNASISVRAQDRYYSSSWSEWASVPCSGGSGGGSGGGSGGGSR |
| | NLPVATPDPGMFPCLHHSQNLLRAVSNMLQKARQTLEFYPCTSEEIDHE |
| | DITKDKTSTVEACLPLELTKNESCLNSRETSFITNGSCLASRKTSFMMALC |
| | LSSIYEDLKMYQVEFKTMNAKLLMDPKRQIFLDQNMLAVIDELMQALN |
| | FNSETVPQKSSLEEPDFYKTKIKLCILLHAFRIRAVTIDRVMSYLNAS |
| SEQ ID NO: 7 | RNLPVATPDPGMFPCLHHSQNLLRAVSNMLQKARQTLEFYPCTSEEIDH |
| P35 sequence | EDITKDKTSTVEACLPLELTKNESCLNSRETSFITNGSCLASRKTSFMMAL |
| | CLSSIYEDLKMYQVEFKTMNAKLLMDPKRQIFLDQNMLAVIDELMQAL |
| | NFNSETVPQKSSLEEPDFYKTKIKLCILLHAFRIRAVTIDRVMSYLNAS |
| SEQ ID NO: 8 | MCHQQLVISWFSLVFLASPLVAIWELKKDVYVVELDWYPDAPGEMVVL |
| P40 sequence | TCDTPEEDGITWTLDQSSEVLGSGKTLTIQVKEFGDAGQYTCHKGGEVL |
| | SHSLLLLHKKEDGIWSTDILKDQKEPKNKTFLRCEAKNYSGRFTCWWLT |
| | TISTDLTFSVKSSRGSSDPQGVTCGAATLSAERVRGDNKEYEYSVECQED |
| | SACPAAEESLPIEVMVDAVHKLKYENYTSSFFIRDIIKPDPPKNLQLKPLK |
| | NSRQVEVSWEYPDTWSTPHSYFSLTFCVQVQGKSKREKKDRVFTDKTS |
| | ATVICRKNASISVRAQDRYYSSSWSEWASVPCSG |
| SEQ ID NO: 9 | GSGGGSGGGSGGGS |
| Linker sequence | |
| SEQ ID NO: 10 | ATAGGCGGCGCATGAGAGAAGCCCAGACCAATTACCTACCCAAAAT |
| srRNA IL-12 | GGAGAAAGTTCACGTTGACATCGAGGAAGACAGCCCATTCCTCAGAG |
| vector sequence 1 | CTTTGCAGCGGAGCTTCCCGCAGTTTGAGGTAGAAGCCAAGCAGGTC |
| | ACTGATAATGACCATGCTAATGCCAGAGCGTTTTCGCATCTGGCTTCA |
| | AAACTGATCGAAACGGAGGTGGACCCATCCGACACGATCCTTGACAT |
| | TGGAAGTGCGCCCGCCCGCAGAATGTATTCTAAGCACAAGTATCATT |
| | GTATCTGTCCGATGAGATGTGCGGAAGATCCGGACAGATTGTATAAG |
| | TATGCAACTAAGCTGAAGAAAAACTGTAAGGAAATAACTGATAAGG |
| | AATTGGACAAGAAAATGAAGGAGCTCGCCGCCGTCATGAGCGACCCT |
| | GACCTGGAAACTGAGACTATGTGCCTCCACGACGACGAGTCGTGTCG |
| | CTACGAAGGGCAAGTCGCTGTTTACCAGGATGTATACGCGGTTGACG |
| | GACCGACAAGTCTCTATCACCAAGCCAATAAGGGAGTTAGAGTCGCC |
| | TACTGGATAGGCTTTGACACCACCCCTTTTATGTTTAAGAACTTGGCT |
| | GGAGCATATCCATCATACTCTACCAACTGGGCCGACGAAACCGTGTT |
| | AACGGCTCGTAACATAGGCCTATGCAGCTCTGACGTTATGGAGCGGT |
| | CACGTAGAGGGATGTCCATTCTTAGAAAGAAGTATTTGAAACCATCC |
| | AACAATGTTCTATTCTCTGTTGGCTCGACCATCTACCACGAGAAGAG |
| | GGACTTACTGAGGAGCTGGCACCTGCCGTCTGTATTTCACTTACGTGG |
| | CAAGCAAAATTACACATGTCGGTGTGAGACTATAGTTAGTTGCGACG |
| | GGTACGTCGTTAAAAGAATAGCTATCAGTCCAGGCCTGTATGGGAAG |
| | CCTTCAGGCTATGCTGCTACGATGCACCGCGAGGGATTCTTGTGCTGC |
| | AAAGTGACAGACACATTGAACGGGGAGAGGGTCTCTTTTCCCGTGTG |
| | CACGTATGTGCCAGCTACATTGTGTGACCAAATGACTGGCATACTGG |
| | CAACAGATGTCAGTGCGGACGACGCGCAAAAACTGCTGGTTGGGCTC |
| | AACCAGCGTATAGTCGTCAACGGTCGCACCCAGAGAAACACCAATAC |
| | CATGAAAAATTACCTTTTGCCCGTAGTGGCCCAGGCATTTGCTAGGTG |
| | GGCAAAGGAATATAAGGAAGATCAAGAAGATGAAAGGCCACTAGGA |
| | CTACGAGATAGACAGTTAGTCATGGGGTGTTGTTGGGCTTTTAGAAG |
| | GCACAAGATAACATCTATTTATAAGCGCCCGGATACCCAAACCATCA |
| | TCAAAGTGAACAGCGATTTCCACTCATTCGTGCTGCCCAGGATAGGC |
| | AGTAACACATTGGAGATCGGGCTGAGAACAAGAATCAGGAAAATGT |
| | TAGAGGAGCACAAGGAGCCGTCACCTCTCATTACCGCCGAGGACGTA |
| | CAAGAAGCTAAGTGCGCAGCCGATGAGGCTAAGGAGGTGCGTGAAG |
| | CCGAGGAGTTGCGCGCAGCTCTACCACCTTTGGCAGCTGATGTTGAG |
| | GAGCCCACTCTGGAAGCCGATGTCGACTTGATGTTACAAGAGGCTGG |
| | GGCCGGCTCAGTGGAGACACCTCGTGGCTTGATAAAGGTTACCAGCT |
| | ACGATGGCGAGGACAAGATCGGCTCTTACGCTGTGCTTTCTCCGCAG |
| | GCTGTACTCAAGAGTGAAAAATTATCTTGCATCCACCCTCTCGCTGAA |
| | CAAGTCATAGTGATAACACACTCTGGCCGAAAAGGGCGTTATGCCGT |
| | GGAACCATACCATGGTAAAGTAGTGGTGCCAGAGGGACATGCAATA |
| | CCCGTCCAGGACTTTCAAGCTCTGAGTGAAAGTGCCACCATTGTGTA |
| | CAACGAACGTGAGTTCGTAAACAGGTACCTGCACCATATTGCCACAC |
| | ATGGAGGAGCGCTGAACACTGATGAAGAATATTACAAAACTGTCAA |
| | GCCCAGCGAGCACGACGGCGAATACCTGTACGACATCGACAGGAAA |
| | CAGTGCGTCAAGAAAGAACTAGTCACTGGGCTAGGGCTCACAGGCG |
| | AGCTGGTGGATCCTCCCTTCCATGAATTCGCCTACGAGAGTCTGAGA |
| | ACACGACCAGCCGCTCCTTACCAAGTACCAACCATAGGGGTGTATGG |
| | CGTGCCAGGATCAGGCAAGTCTGGCATCATTAAAAGCGCAGTCACCA |
| | AAAAAGATCTAGTGGTGAGCGCCAAGAAAGAAAACTGTGCAGAAAT |
| | TATAAGGGACGTCAAGAAAATGAAAGGGCTGGACGTCAATGCCAGA |
| | ACTGTGGACTCAGTGCTCTTGAATGGATGCAAACACCCCGTAGAGAC |
| | CCTGTATATTGACGAAGCTTTTGCTTGTCATGCAGGTACTCTCAGAGC |
| | GCTCATAGCCATTATAAGACCTAAAAAGGCAGTGCTCTGCGGGGATC |
| | CCAAACAGTGCGGTTTTTTTAACATGATGTGCCTGAAAGTGCATTTTA |
| | ACCACGAGATTTGCACACAAGTCTTCCACAAAAGCATCTCTCGCCGT |
| | TGCACTAAATCTGTGACTTCGGTCGTCTCAACCTTGTTTTACGACAAA |
| | AAAATGAGAACGACGAATCCGAAAGAGACTAAGATTGTGATTGACA |
| | CTACCGGCAGTACCAAACCTAAGCAGGACGATCTCATTCTCACTTGTT |
| | TCAGAGGGTGGGTGAAGCAGTTGCAAATAGATTACAAAGGCAACGA |
| | AATAATGACGGCAGCTGCCTCTCAAGGGCTGACCCGTAAAGGTGTGT |
| | ATGCCGTTCGGTACAAGGTGAATGAAAATCCTCTGTACGCACCCACC |
| | TCAGAACATGTGAACGTCCTACTGACCCGCACGGAGGACCGCATCGT |
| | GTGGAAAACACTAGCCGGCGACCCATGGATAAAAACACTGACTGCC |
| | AAGTACCCTGGGAATTTCACTGCCACGATAGAGGAGTGGCAAGCAGA |
| | GCATGATGCCATCATGAGGCACATCTTGGAGAGACCGGACCCTACCG |
| | ACGTCTTCCAGAATAAGGCAAACGTGTGTTGGGCCAAGGCTTTAGTG |
| | CCGGTGCTGAAGACCGCTGGCATAGACATGACCACTGAACAATGGAA |
| | CACTGTGGATTATTTTGAAACGGACAAAGCTCACTCAGCAGAGATAG |
| | TATTGAACCAACTATGCGTGAGGTTCTTTGGACTCGATCTGGACTCCG |
| | GTCTATTTTCTGCACCCACTGTTCCGTTATCCATTAGGAATAATCACT |
| | GGGATAACTCCCCGTCGCCTAACATGTACGGGCTGAATAAAGAAGTG |
| | GTCCGTCAGCTCTCTCGCAGGTACCCACAACTGCCTCGGGCAGTTGCC |
| | ACTGGAAGAGTCTATGACATGAACACTGGTACACTGCGCAATTATGA |
| | TCCGCGCATAAACCTAGTACCTGTAAACAGAAGACTGCCTCATGCTT |
| | TAGTCCTCCACCATAATGAACACCCACAGAGTGACTTTTCTTCATTCG |
| | TCAGCAAATTGAAGGGCAGAACTGTCCTGGTGGTCGGGGAAAAGTTG |
| | TCCGTCCCAGGCAAAATGGTTGACTGGTTGTCAGACCGGCCTGAGGC |
| | TACCTTCAGAGCTCGGCTGGATTTAGGCATCCCAGGTGATGTGCCCA |
| | AATATGACATAATATTTGTTAATGTGAGGACCCCATATAAATACCAT |
| | CACTATCAGCAGTGTGAAGACCATGCCATTAAGCTTAGCATGTTGAC |
| | CAAGAAAGCTTGTCTGCATCTGAATCCCGGCGGAACCTGTGTCAGCA |
| | TAGGTTATGGTTACGCTGACAGGGCCAGCGAAAGCATCATTGGTGCT |
| | ATAGCGCGGCAGTTCAAGTTTTCCCGGGTATGCAAACCGAAATCCTC |
| | ACTTGAAGAGACGGAAGTTCTGTTTGTATTCATTGGGTACGATCGCA |
| | AGGCCCGTACGCACAATCCTTACAAGCTTTCATCAACCTTGACCAAC |
| | ATTTATACAGGTTCCAGACTCCACGAAGCCGGATGTGCACCCTCATA |
| | TCATGTGGTGCGAGGGGATATTGCCACGGCCACCGAAGGAGTGATTA |
| | TAAATGCTGCTAACAGCAAAGGACAACCTGGCGGAGGGGTGTGCGG |
| | AGCGCTGTATAAGAAATTCCCGGAAAGCTTCGATTTACAGCCGATCG |
| | AAGTAGGAAAAGCGCGACTGGTCAAAGGTGCAGCTAAACATATCATT |
| | CATGCCGTAGGACCAAACTTCAACAAAGTTTCGGAGGTTGAAGGTGA |
| | CAAACAGTTGGCAGAGGCTTATGAGTCCATCGCTAAGATTGTCAACG |
| | ATAACAATTACAAGTCAGTAGCGATTCCACTGTTGTCCACCGGCATCT |
| | TTTCCGGGAACAAAGATCGACTAACCCAATCATTGAACCATTTGCTG |
| | ACAGCTTTAGACACCACTGATGCAGATGTAGCCATATACTGCAGGGA |
| | CAAGAAATGGGAAATGACTCTCAAGGAAGCAGTGGCTAGGAGAGAA |
| | GCAGTGGAGGAGATATGCATATCCGACGACTCTTCAGTGACAGAACC |
| | TGATGCAGAGCTGGTGAGGGTGCATCCGAAGAGTTCTTTGGCTGGAA |
| | GGAAGGGCTACAGCACAAGCGATGGCAAAACTTTCTCATATTTGGAA |
| | GGGACCAAGTTTCACCAGGCGGCCAAGGATATAGCAGAAATTAATGC |
| | CATGTGGCCCGTTGCAACGGAGGCCAATGAGCAGGTATGCATGTATA |
| | TCCTCGGAGAAAGCATGAGCAGTATTAGGTCGAAATGCCCCGTCGAA |
| | GAGTCGGAAGCCTCCACACCACCTAGCACGCTGCCTTGCTTGTGCAT |
| | CCATGCCATGACTCCAGAAAGAGTACAGCGCCTAAAAGCCTCACGTC |
| | CAGAACAAATTACTGTGTGCTCATCCTTTCCATTGCCGAAGTATAGAA |
| | TCACTGGTGTGCAGAAGATCCAATGCTCCCAGCCTATATTGTTCTCAC |
| | CGAAAGTGCCTGCGTATATTCATCCAAGGAAGTATCTCGTGGAAACA |
| | CCACCGGTAGACGAGACTCCGGAGCCATCGGCAGAGAACCAATCCA |
| | CAGAGGGGACACCTGAACAACCACCACTTATAACCGAGGATGAGAC |
| | CAGGACTAGAACGCCTGAGCCGATCATCATCGAAGAGGAAGAAGAG |
| | GATAGCATAAGTTTGCTGTCAGATGGCCCGACCCACCAGGTGCTGCA |
| | AGTCGAGGCAGACATTCACGGGCCGCCCTCTGTATCTAGCTCATCCT |
| | GGTCCATTCCTCATGCATCCGACTTTGATGTGGACAGTTTATCCATAC |
| | TTGACACCCTGGAGGGAGCTAGCGTGACCAGCGGGGCAACGTCAGCC |
| | GAGACTAACTCTTACTTCGCAAAGAGTATGGAGTTTCTGGCGCGACC |
| | GGTGCCTGCGCCTCGAACAGTATTCAGGAACCCTCCACATCCCGCTC |
| | CGCGCACAAGAACACCGTCACTTGCACCCAGCAGGGCCTGCTCGAGA |
| | ACCAGCCTAGTTTCCACCCCGCCAGGCGTGAATAGGGTGATCACTAG |
| | AGAGGAGCTCGAGGCGCTTACCCCGTCACGCACTCCTAGCAGGTCGG |
| | TCTCGAGAACCAGCCTGGTCTCCAACCCGCCAGGCGTAAATAGGGTG |
| | ATTACAAGAGAGGAGTTTGAGGCGTTCGTAGCACAACAACAATGACG |
| | GTTTGATGCGGGTGCATACATCTTTTCCTCCGACACCGGTCAAGGGCA |
| | TTTACAACAAAAATCAGTAAGGCAAACGGTGCTATCCGAAGTGGTGT |
| | TGGAGAGGACCGAATTGGAGATTTCGTATGCCCCGCGCCTCGACCAA |
| | GAAAAAGAAGAATTACTACGCAAGAAATTACAGTTAAATCCCACACC |
| | TGCTAACAGAAGCAGATACCAGTCCAGGAAGGTGGAGAACATGAAA |
| | GCCATAACAGCTAGACGTATTCTGCAAGGCCTAGGGCATTATTTGAA |
| | GGCAGAAGGAAAAGTGGAGTGCTACCGAACCCTGCATCCTGTTCCTT |
| | TGTATTCATCTAGTGTGAACCGTGCCTTTTCAAGCCCCAAGGTCGCAG |
| | TGGAAGCCTGTAACGCCATGTTGAAAGAGAACTTTCCGACTGTGGCT |
| | TCTTACTGTATTATTCCAGAGTACGATGCCTATTTGGACATGGTTGAC |
| | GGAGCTTCATGCTGCTTAGACACTGCCAGTTTTTGCCCTGCAAAGCTG |
| | CGCAGCTTTCCAAAGAAACACTCCTATTTGGAACCCACAATACGATC |
| | GGCAGTGCCTTCAGCGATCCAGAACACGCTCCAGAACGTCCTGGCAG |
| | CTGCCACAAAAAGAAATTGCAATGTCACGCAAATGAGAGAATTGCCC |
| | GTATTGGATTCGGCGGCCTTTAATGTGGAATGCTTCAAGAAATATGC |
| | GTGTAATAATGAATATTGGGAAACGTTTAAAGAAAACCCCATCAGGC |
| | TTACTGAAGAAAACGTGGTAAATTACATTACCAAATTAAAAGGACCA |
| | AAAGCTGCTGCTCTTTTTGCGAAGACACATAATTTGAATATGTTGCAG |
| | GACATACCAATGGACAGGTTTGTAATGGACTTAAAGAGAGACGTGAA |
| | AGTGACTCCAGGAACAAAACATACTGAAGAACGGCCCAAGGTACAG |
| | GTGATCCAGGCTGCCGATCCGCTAGCAACAGCGTATCTGTGCGGAAT |
| | CCACCGAGAGCTGGTTAGGAGATTAAATGCGGTCCTGCTTCCGAACA |
| | TTCATACACTGTTTGATATGTCGGCTGAAGACTTTGACGCTATTATAG |
| | CCGAGCACTTCCAGCCTGGGGATTGTGTTCTGGAAACTGACATCGCG |
| | TCGTTTGATAAAAGTGAGGACGACGCCATGGCTCTGACCGCGTTAAT |
| | GATTCTGGAAGACTTAGGTGTGGACGCAGAGCTGTTGACGCTGATTG |
| | AGGCGGCTTTCGGCGAAATTTCATCAATACATTTGCCCACTAAAACT |
| | AAATTTAAATTCGGAGCCATGATGAAATCTGGAATGTTCCTCACACT |
| | GTTTGTGAACACAGTCATTAACATTGTAATCGCAAGCAGAGTGTTGA |
| | GAGAACGGCTAACCGGATCACCATGTGCAGCATTCATTGGAGATGAC |
| | AATATCGTGAAAGGAGTCAAATCGGACAAATTAATGGCAGACAGGT |
| | GCGCCACCTGGTTGAATATGGAAGTCAAGATTATAGATGCTGTGGTG |
| | GGCGAGAAAGCGCCTTATTTCTGTGGAGGGTTTATTTTGTGTGACTCC |
| | GTGACCGGCACAGCGTGCCGTGTGGCAGACCCCCTAAAAAGGCTGTT |
| | TAAGCTTGGCAAACCTCTGGCAGCAGACGATGAACATGATGATGACA |
| | GGAGAAGGGCATTGCATGAAGAGTCAACACGCTGGAACCGAGTGGG |
| | TATTCTTTCAGAGCTGTGCAAGGCAGTAGAATCAAGGTATGAAACCG |
| | TAGGAACTTCCATCATAGTTATGGCCATGACTACTCTAGCTAGCAGTG |
| | TTAAATCATTCAGCTACCTGAGAGGGGCCcctataactctctacggctaacctgaatgga |
| | ctacgacatagtctagtccgccaagATGTGCCACCAGCAGCTGGTCATCAGCTGGTT |
| | CAGCCTCGTTTTCCTCGCCTCGCCGCTGGTCGCCATATGGGAGCTCAA |
| | GAAGGACGTATACGTGGTGGAGCTGGACTGGTACCCCGACGCGCCGG |
| | GCGAGATGGTCGTCCTGACGTGCGACACGCCGGAGGAGGACGGCAT |
| | CACGTGGACGCTGGACCAGTCCAGCGAGGTCCTCGGCTCCGGCAAGA |
| | CGCTGACGATCCAGGTCAAGGAGTTCGGCGACGCGGGCCAGTACACG |
| | TGCCACAAGGGCGGCGAGGTCCTGAGCCACTCCCTCCTCCTGCTACA |
| | CAAGAAGGAGGACGGGATCTGGAGCACGGACATCCTCAAGGACCAG |
| | AAGGAGCCGAAGAACAAGACCTTCCTGCGCTGCGAGGCGAAGAATT |
| | ACTCGGGCCGGTTCACGTGCTGGTGGCTCACCACGATCAGCACGGAC |
| | CTGACGTTCTCGGTCAAGTCGTCGCGGGGCTCGTCGGACCCCCAGGG |
| | GGTGACCTGCGGCGCGGCGACGCTGTCGGCGGAGCGGGTGCGGGGC |
| | GACAACAAGGAGTACGAGTACTCGGTCGAGTGCCAGGAGGACTCGG |
| | CGTGCCCGGCGGCGGAGGAGTCGCTGCCGATCGAGGTGATGGTCGAC |
| | GCGGTCCACAAGCTGAAGTACGAGAACTACACGTCGTCGTTCTTCAT |
| | CCGGGACATCATCAAGCCGGACCCGCCGAAGAACCTGCAGCTGAAG |
| | CCGCTGAAGAACTCGCGGCAGGTCGAGGTCTCGTGGGAGTACCCGGA |
| | CACGTGGTCGACGCCGCACTCGTACTTCTCGCTGACGTTCTGCGTCCA |
| | AGTGCAGGGCAAGTCGAAGCGGGAGAAGAAGGACCGGGTGTTCACC |
| | GACAAGACGAGCGCGACGGTGATCTGCCGGAAGAACGCGTCGATCT |
| | CGGTGCGGGCGCAGGACCGGTACTACTCGTCGTCGTGGTCGGAGTGG |
| | GCGTCGGTGCCGTGCAGCGGCGGAAGCGGCGGCGGCAGCGGCGGAG |
| | GCTCTGGCGGCGGCTCCCGGAACCTGCCGGTGGCGACGCCGGACCCG |
| | GGGATGTTCCCGTGCCTGCACCACAGCCAGAACCTGCTGCGGGCGGT |
| | GTCGAACATGCTGCAGAAGGCGCGGCAGACGCTGGAGTTCTACCCGT |
| | GCACGAGCGAGGAGATCGACCACGAGGACATCACGAAGGACAAGAC |
| | CAGCACGGTGGAGGCGTGCCTGCCGCTGGAGCTGACGAAGAACGAG |
| | TCGTGCCTGAACTCGAGGGAGACGTCGTTCATCACGAACGGGTCGTG |
| | CCTGGCGTCGCGGAAGACGTCGTTCATGATGGCGCTGTGCCTGTCGT |
| | CGATCTACGAGGACCTGAAGATGTACCAGGTGGAGTTCAAGACGATG |
| | AACGCGAAGCTGCTGATGGACCCGAAGCGGCAGATCTTCCTCGACCA |
| | GAACATGCTGGCGGTGATCGACGAGCTCATGCAGGCGCTCAACTTCA |
| | ACAGCGAGACGGTGCCGCAGAAGTCGTCGCTCGAGGAGCCGGACTTC |
| | TACAAGACGAAGATCAAGCTCTGCATCCTGCTGCACGCTTTCCGGAT |
| | CCGGGCGGTGACGATCGACCGGGTGATGTCGTACCTGAACGCTTCGtg |
| | ataaggcgcgcccacccagcGGCCGCATACAGCAGCAATTGGCAAGCTGCTTAC |
| | ATAGAACTCGCGGCGATTGGCATGCCGCCTTAAAATTTTTATTTTATT |
| | TTTCTTTTCTTTTCCGAATCGGATTTTGTTTTTAATATTTCAAAAAAAA |
| | AAAAAAAAAAAAAAAAAAAAAAAAAAAAAAAAAGAAGAGCTAGGG |
| | ATAAcagggtaatTGAGCAAAAGGCCAGCAAAAGGCCAGGAACCGTAAA |
| | AAGGCCGCGTTGCTGGCGTTTTTCCATAGGCTCCGCCCCCCTGACGAG |
| | CATCACAAAAATCGACGCTCAAGTCAGAGGTGGCGAAACCCGACAG |
| | GACTATAAAGATACCAGGCGTTTCCCCCTGGAAGCTCCCTCGTGCGC |
| | TCTCCTGTTCCGACCCTGCCGCTTACCGGATACCTGTCCGCCTTTCTCC |
| | CTTCGGGAAGCGTGGCGCTTTCTCATAGCTCACGCTGTAGGTATCTCA |
| | GTTCGGTGTAGGTCGTTCGCTCCAAGCTGGGCTGTGTGCACGAACCC |
| | CCCGTTCAGCCCGACCGCTGCGCCTTATCCGGTAACTATCGTCTTGAG |
| | TCCAACCCGGTAAGACACGACTTATCGCCACTGGCAGCAGCCACTGG |
| | TAACAGGATTAGCAGAGCGAGGTATGTAGGCGGTGCTACAGAGTTCT |
| | TGAAGTGGTGGCCTAACTACGGCTACACTAGAAGAACAGTATTTGGT |
| | ATCTGCGCTCTGCTGAAGCCAGTTACCTTCGGAAAAAGAGTTGGTAG |
| | CTCTTGATCCGGCAAACAAACCACCGCTGGTAGCGGTGGTTTTTTTGT |
| | TTGCAAGCAGCAGATTACGCGCAGAAAAAAAGGATCTCAAGAAGAT |
| | CCTTTGATCTTTTCTACGGGGTCTGACGCTCAGTGGAACGAAAACTCA |
| | CGTTAAGGGATTTTGGTCATGAGATTATCAAAAAGGATCTTCACCTA |
| | GATCCTTTTAAATTAAAAATGAAGTTTTAAATCAATCTAAAGTATATA |
| | TGAGTAAACTTGGTCTGACAGTTAGAAAAACTCATCGAGCATCAAAT |
| | GAAACTGCAATTTATTCATATCAGGATTATCAATACCATATTTTTGAA |
| | AAAGCCGTTTCTGTAATGAAGGAGAAAACTCACCGAGGCAGTTCCAT |
| | AGGATGGCAAGATCCTGGTATCGGTCTGCGATTCCGACTCGTCCAAC |
| | ATCAATACAACCTATTAATTTCCCCTCGTCAAAAATAAGGTTATCAAG |
| | TGAGAAATCACCATGAGTGACGACTGAATCCGGTGAGAATGGCAAA |
| | AGTTTATGCATTTCTTTCCAGACTTGTTCAACAGGCCAGCCATTACGC |
| | TCGTCATCAAAATCACTCGCATCAACCAAACCGTTATTCATTCGTGAT |
| | TGCGCCTGAGCGAGACGAAATACGCGATCGCTGTTAAAAGGACAATT |
| | ACAAACAGGAATCGAATGCAACCGGCGCAGGAACACTGCCAGCGCA |
| | TCAACAATATTTTCACCTGAATCAGGATATTCTTCTAATACCTGGAAT |
| | GCTGTTTTCCCAGGGATCGCAGTGGTGAGTAACCATGCATCATCAGG |
| | AGTACGGATAAAATGCTTGATGGTCGGAAGAGGCATAAATTCCGTCA |
| | GCCAGTTTAGTCTGACCATCTCATCTGTAACATCATTGGCAACGCTAC |
| | CTTTGCCATGTTTCAGAAACAACTCTGGCGCATCGGGCTTCCCATACA |
| | ATCGATAGATTGTCGCACCTGATTGCCCGACATTATCGCGAGCCCATT |
| | TATACCCATATAAATCAGCATCCATGTTGGAATTTAATCGCGGCCTAG |
| | AGCAAGACGTTTCCCGTTGAATATGGCTCATACTCTTCCTTTTTCAAT |
| | ATTATTGAAGCATTTATCAGGGTTATTGTCTCATGAGCGGATACATAT |
| | TTGAATGTATTTAGAAAAATAAACAAATAGGGGTTCCGCGCACATTT |
| | CCCCGAAAAGTGCCACCTGACGTCTAAGAAACCATTATTATCATGAC |
| | ATTAACCTATAAAAATAGGCGTATCACGAGGCCCTTTCGTCtagggataaC |
| | AGGGTAATTAATACGACTCACTATAG |
| SEQ ID NO: 11 | ataggcggcgcatgagagaagcccagaccaattacctacccaaaatggagaaagttcacgttgacatcgagga |
| srRNA IL-12 | agacagcccattcctcagagctttgcagcggagcttcccgcagtttgaggtagaagccaagcaggtcactgat |
| sequence 2 | aatgaccatgctaatgccagagcgttttcgcatctggcttcaaaactgatcgaaacggaggtggacccatccg |
| | acacgatccttgacattggaagtgcgcccgcccgcagaatgtattctaagcacaagtatcattgtatctgtcc |
| | gatgagatgtgcggaagatccggacagattgtataagtatgcaactaagctgaagaaaaactgtaaggaaata |
| | actgataaggaattggacaagaaaatgaaggagctcgccgccgtcatgagcgaccctgacctggaaactgaga |
| | ctatgtgcctccacgacgacgagtcgtgtcgctacgaagggcaagtcgctgtttaccaggatgtatacgcggt |
| | tgacggaccgacaagtctctatcaccaagccaataagggagttagagtcgcctactggataggctttgacacc |
| | accccttttatgtttaagaacttggctggagcatatccatcatactctaccaactgggccgacgaaaccgtgt |
| | taacggctcgtaacataggcctatgcagctctgacgttatggagcggtcacgtagagggatgtccattcttag |
| | aaagaagtatttgaaaccatccaacaatgttctattctctgttggctcgaccatctaccacgagaagagggac |
| | ttactgaggagctggcacctgccgtctgtatttcacttacgtggcaagcaaaattacacatgtcggtgtgaga |
| | ctatagttagttgcgacgggtacgtcgttaaaagaatagctatcagtccaggcctgtatgggaagccttcagg |
| | ctatgctgctacgatgcaccgcgagggattcttgtgctgcaaagtgacagacacattgaacggggagagggtc |
| | tcttttcccgtgtgcacgtatgtgccagctacattgtgtgaccaaatgactggcatactggcaacagatgtca |
| | gtgcggacgacgcgcaaaaactgctggttgggctcaaccagcgtatagtcgtcaacggtcgcacccagagaaa |
| | caccaataccatgaaaaattaccttttgcccgtagtggcccaggcatttgctaggtgggcaaaggaatataag |
| | gaagatcaagaagatgaaaggccactaggactacgagatagacagttagtcatggggtgttgttgggctttta |
| | gaaggcacaagataacatctatttataagcgcccggatacccaaaccatcatcaaagtgaacagcgatttcca |
| | ctcattcgtgctgcccaggataggcagtaacacattggagatcgggctgagaacaagaatcaggaaaatgtta |
| | gaggagcacaaggagccgtcacctctcattaccgccgaggacgtacaagaagctaagtgcgcagccgatgag |
| | gctaaggaggtgcgtgaagccgaggagttgcgcgcagctctaccacctttggcagctgatgttgaggagccca |
| | ctctggaagccgatgtcgacttgatgttacaagaggctggggccggctcagtggagacacctcgtggcttgat |
| | aaaggttaccagctacgatggcgaggacaagatcggctcttacgctgtgctttctccgcaggctgtactcaag |
| | agtgaaaaattatcttgcatccaccctctcgctgaacaagtcatagtgataacacactctggccgaaaagggc |
| | gttatgccgtggaaccataccatggtaaagtagtggtgccagagggacatgcaatacccgtccaggactttca |
| | agctctgagtgaaagtgccaccattgtgtacaacgaacgtgagttcgtaaacaggtacctgcaccatattgcc |
| | acacatggaggagcgctgaacactgatgaagaatattacaaaactgtcaagcccagcgagcacgacggcgaat |
| | acctgtacgacatcgacaggaaacagtgcgtcaagaaagaactagtcactgggctagggctcacaggcgagct |
| | ggtggatcctcccttccatgaattcgcctacgagagtctgagaacacgaccagccgctccttaccaagtacca |
| | accataggggtgtatggcgtgccaggatcaggcaagtctggcatcattaaaagcgcagtcaccaaaaaagatc |
| | tagtggtgagcgccaagaaagaaaactgtgcagaaattataagggacgtcaagaaaatgaaagggctggacgt |
| | caatgccagaactgtggactcagtgctcttgaatggatgcaaacaccccgtagagaccctgtatattgacgaa |
| | gcttttgcttgtcatgcaggtactctcagagcgctcatagccattataagacctaaaaaggcagtgctctgcg |
| | gggatcccaaacagtgcggtttttttaacatgatgtgcctgaaagtgcattttaaccacgagatttgcacaca |
| | agtcttccacaaaagcatctctcgccgttgcactaaatctgtgacttcggtcgtctcaaccttgttttacgac |
| | aaaaaaatgagaacgacgaatccgaaagagactaagattgtgattgacactaccggcagtaccaaacctaagc |
| | aggacgatctcattctcacttgtttcagaggggggtgaagcagttgcaaatagattacaaaggcaacgaaata |
| | atgacggcagctgcctctcaagggctgacccgtaaaggtgtgtatgccgttcggtacaaggtgaatgaaaatc |
| | ctctgtacgcacccacctcagaacatgtgaacgtcctactgacccgcacggaggaccgcatcgtgtggaaaac |
| | actagccggcgacccatggataaaaacactgactgccaagtaccctgggaatttcactgccacgatagaggag |
| | tggcaagcagagcatgatgccatcatgaggcacatcttggagagaccggaccctaccgacgtcttccagaata |
| | aggcaaacgtgtgttgggccaaggctttagtgccggtgctgaagaccgctggcatagacatgaccactgaaca |
| | atggaacactgtggattattttgaaacggacaaagctcactcagcagagatagtattgaaccaactatgcgtg |
| | aggttctttggactcgatctggactccggtctattttctgcacccactgttccgttatccattaggaataatc |
| | actgggataactccccgtcgcctaacatgtacgggctgaataaagaagtggtccgtcagctctctcgcaggta |
| | cccacaactgcctcgggcagttgccactggaagagtctatgacatgaacactggtacactgcgcaattatgat |
| | ccgcgcataaacctagtacctgtaaacagaagactgcctcatgctttagtcctccaccataatgaacacccac |
| | agagtgacttttcttcattcgtcagcaaattgaagggcagaactgtcctggtggtcggggaaaagttgtccgt |
| | cccaggcaaaatggttgactggttgtcagaccggcctgaggctaccttcagagctcggctggatttaggcatc |
| | ccaggtgatgtgcccaaatatgacataatatttgttaatgtgaggaccccatataaataccatcactatcagc |
| | agtgtgaagaccatgccattaagcttagcatgttgaccaagaaagcttgtctgcatctgaatcccggcggaac |
| | ctgtgtcagcataggttatggttacgctgacagggccagcgaaagcatcattggtgctatagcgcggcagttc |
| | aagttttcccgggtatgcaaaccgaaatcctcacttgaagagacggaagttctgtttgtattcattgggtacg |
| | atcgcaaggcccgtacgcacaatccttacaagctttcatcaaccttgaccaacatttatacaggttccagact |
| | ccacgaagccggatgtgcaccctcatatcatgtggtgcgaggggatattgccacggccaccgaaggagtgatt |
| | ataaatgctgctaacagcaaaggacaacctggcggaggggtgtgcggagcgctgtataagaaattcccggaaa |
| | gcttcgatttacagccgatcgaagtaggaaaagcgcgactggtcaaaggtgcagctaaacatatcattcatgc |
| | cgtaggaccaaacttcaacaaagtttcggaggttgaaggtgacaaacagttggcagaggcttatgagtccatc |
| | gctaagattgtcaacgataacaattacaagtcagtagcgattccactgttgtccaccggcatcttttccggga |
| | acaaagatcgactaacccaatcattgaaccatttgctgacagctttagacaccactgatgcagatgtagccat |
| | atactgcagggacaagaaatgggaaatgactctcaaggaagcagtggctaggagagaagcagtggaggagata |
| | tgcatatccgacgactcttcagtgacagaacctgatgcagagctggtgagggtgcatccgaagagttctttgg |
| | ctggaaggaagggctacagcacaagcgatggcaaaactttctcatatttggaagggaccaagtttcaccaggc |
| | ggccaaggatatagcagaaattaatgccatgtggcccgttgcaacggaggccaatgagcaggtatgcatgtat |
| | atcctcggagaaagcatgagcagtattaggtcgaaatgccccgtcgaagagtcggaagcctccacaccaccta |
| | gcacgctgccttgcttgtgcatccatgccatgactccagaaagagtacagcgcctaaaagcctcacgtccag |
| | aacaaattactgtgtgctcatcctttccattgccgaagtatagaatcactggtgtgcagaagatccaatgctc |
| | ccagcctatattgttctcaccgaaagtgcctgcgtatattcatccaaggaagtatctcgtggaaacaccacc |
| | ggtagacgagactccggagccatcggcagagaaccaatccacagaggggacacctgaacaaccaccacttata |
| | accgaggatgagaccaggactagaacgcctgagccgatcatcatcgaagaggaagaagaggatagcataagtt |
| | tgctgtcagatggcccgacccaccaggtgctgcaagtcgaggcagacattcacgggccgccctctgtatctag |
| | ctcatcctggtccattcctcatgcatccgactttgatgtggacagtttatccatacttgacaccctggaggga |
| | gctagcgtgaccagcggggcaacgtcagccgagactaactcttacttcgcaaagagtatggagtttctggcgc |
| | gaccggtgcctgcgcctcgaacagtattcaggaaccctccacatcccgctccgcgcacaagaacaccgtcact |
| | tgcacccagcagggcctgctcgagaaccagcctagtttccaccccgccaggcgtgaatagggtgatcactaga |
| | gaggagctcgaggcgcttaccccgtcacgcactcctagcaggtcggtctcgagaaccagcctggtctccaacc |
| | cgccaggcgtaaatagggtgattacaagagaggagtttgaggcgttcgtagcacaacaacaatgacggtttga |
| | tgcgggtgcatacatcttttcctccgacaccggtcaagggcatttacaacaaaaatcagtaaggcaaacggtg |
| | ctatccgaagtggtgttggagaggaccgaattggagatttcgtatgccccgcgcctcgaccaagaaaaagaag |
| | aattactacgcaagaaattacagttaaatcccacacctgctaacagaagcagataccagtccaggaaggtgga |
| | gaacatgaaagccataacagctagacgtattctgcaaggcctagggcattatttgaaggcagaaggaaaagtg |
| | gagtgctaccgaaccctgcatcctgttcctttgtattcatctagtgtgaaccgtgccttttcaagccccaagg |
| | tcgcagtggaagcctgtaacgccatgttgaaagagaactttccgactgtggcttcttactgtattattccaga |
| | gtacgatgcctatttggacatggttgacggagcttcatgctgcttagacactgccagtttttgccctgcaaag |
| | ctgcgcagctttccaaagaaacactcctatttggaacccacaatacgatcggcagtgccttcagcgatccaga |
| | acacgctccagaacgtcctggcagctgccacaaaaagaaattgcaatgtcacgcaaatgagagaattgcccgt |
| | attggattcggcggcctttaatgtggaatgcttcaagaaatatgcgtgtaataatgaatattgggaaacgttt |
| | aaagaaaaccccatcaggcttactgaagaaaacgtggtaaattacattaccaaattaaaaggaccaaaagct |
| | gctgctctttttgcgaagacacataatttgaatatgttgcaggacataccaatggacaggtttgtaatggac |
| | ttaaagagagacgtgaaagtgactccaggaacaaaacatactgaagaacggcccaaggtacaggtgatccagg |
| | ctgccgatccgctagcaacagcgtatctgtgcggaatccaccgagagctggttaggagattaaatgcggtcct |
| | gcttccgaacattcatacactgtttgatatgtcggctgaagactttgacgctattatagccgagcacttccag |
| | cctggggattgtgttctggaaactgacatcgcgtcgtttgataaaagtgaggacgacgccatggctctgaccg |
| | cgttaatgattctggaagacttaggtgtggacgcagagctgttgacgctgattgaggcggctttcggcgaaat |
| | ttcatcaatacatttgcccactaaaactaaatttaaattcggagccatgatgaaatctggaatgttcctcaca |
| | ctgtttgtgaacacagtcattaacattgtaatcgcaagcagagtgttgagagaacggctaaccggatcaccat |
| | gtgcagcattcattggagatgacaatatcgtgaaaggagtcaaatcggacaaattaatggcagacaggtgcgc |
| | cacctggttgaatatggaagtcaagattatagatgctgtggtgggcgagaaagcgccttatttctgtgga |
| | gggtttattttgtgtgactccgtgaccggcacagcgtgccgtgtggcagaccccctaaaaaggctgtttaag |
| | cttggcaaacctctggcagcagacgatgaacatgatgatgacaggagaagggcattgcatgaagagtcaacac |
| | gctggaaccgagtgggtattctttcagagctgtgcaaggcagtagaatcaaggtatgaaaccgtaggaacttc |
| | catcatagttatggccatgactactctagctagcagtgttaaatcattcagctacctgagaggggcccctat |
| | aactctctacggctaacctgaatggactacgacatagtctagtccgccaagATGTGCCACCAGCAGCTGGTC |
| | ATCAGCTGGTTCAGCCTCGTTTTCCTCGCCTCGCCGCTGGTCGCCATATGG |
| | GAGCTCAAGAAGGACGTATACGTGGTGGAGCTGGACTGGTACCCCGA |
| | CGCGCCGGGCGAGATGGTCGTCCTGACGTGCGACACGCCGGAGGAG |
| | GACGGCATCACGTGGACGCTGGACCAGTCCAGCGAGGTCCTCGGCTC |
| | CGGCAAGACGCTGACGATCCAGGTCAAGGAGTTCGGCGACGCGGGC |
| | CAGTACACGTGCCACAAGGGCGGCGAGGTCCTGAGCCACTCCCTCCT |
| | CCTGCTACACAAGAAGGAGGACGGGATCTGGAGCACGGACATCCTC |
| | AAGGACCAGAAGGAGCCGAAGAACAAGACCTTCCTGCGCTGCGAGG |
| | CGAAGAATTACTCGGGCCGGTTCACGTGCTGGTGGCTCACCACGATC |
| | AGCACGGACCTGACGTTCTCGGTCAAGTCGTCGCGGGGCTCGTCGGA |
| | CCCCCAGGGGGTGACCTGCGGCGCGGCGACGCTGTCGGCGGAGCGG |
| | GTGCGGGGCGACAACAAGGAGTACGAGTACTCGGTCGAGTGCCAGG |
| | AGGACTCGGCGTGCCCGGCGGCGGAGGAGTCGCTGCCGATCGAGGT |
| | GATGGTCGACGCGGTCCACAAGCTGAAGTACGAGAACTACACGTCGT |
| | CGTTCTTCATCCGGGACATCATCAAGCCGGACCCGCCGAAGAACCTG |
| | CAGCTGAAGCCGCTGAAGAACTCGCGGCAGGTCGAGGTCTCGTGGGA |
| | GTACCCGGACACGTGGTCGACGCCGCACTCGTACTTCTCGCTGACGTT |
| | CTGCGTCCAAGTGCAGGGCAAGTCGAAGCGGGAGAAGAAGGACCGG |
| | GTGTTCACCGACAAGACGAGCGCGACGGTGATCTGCCGGAAGAACG |
| | CGTCGATCTCGGTGCGGGCGCAGGACCGGTACTACTCGTCGTCGTGG |
| | TCGGAGTGGGCGTCGGTGCCGTGCAGCGGCGGAAGCGGCGGCGGCA |
| | GCGGCGGAGGCTCTGGCGGCGGCTCCCGGAACCTGCCGGTGGCGACG |
| | CCGGACCCGGGGATGTTCCCGTGCCTGCACCACAGCCAGAACCTGCT |
| | GCGGGCGGTGTCGAACATGCTGCAGAAGGCGCGGCAGACGCTGGAG |
| | TTCTACCCGTGCACGAGCGAGGAGATCGACCACGAGGACATCACGAA |
| | GGACAAGACCAGCACGGTGGAGGCGTGCCTGCCGCTGGAGCTGACG |
| | AAGAACGAGTCGTGCCTGAACTCGAGGGAGACGTCGTTCATCACGAA |
| | CGGGTCGTGCCTGGCGTCGCGGAAGACGTCGTTCATGATGGCGCTGT |
| | GCCTGTCGTCGATCTACGAGGACCTGAAGATGTACCAGGTGGAGTTC |
| | AAGACGATGAACGCGAAGCTGCTGATGGACCCGAAGCGGCAGATCT |
| | TCCTCGACCAGAACATGCTGGCGGTGATCGACGAGCTCATGCAGGCG |
| | CTCAACTTCAACAGCGAGACGGTGCCGCAGAAGTCGTCGCTCGAGGA |
| | GCCGGACTTCTACAAGACGAAGATCAAGCTCTGCATCCTGCTGCACG |
| | CTTTCCGGATCCGGGCGGTGACGATCGACCGGGTGATGTCGTACCTG |
| | AACGCTTCGtgataaggcgcgcccacccagcggccgctatgttacgtgcaaaggtgattgtcaccccccg |
| | aaagaccatattgtgacacaccctcagtatcacgcccaaacatttacagccgcggtgtcaaaaaccgcgtgg |
| | acgtggttaacatccctgctgggaggatcagccgtaattattataattggcttggtgctggctactattgtgg |
| | ccatgtacgtgctgaccaaccagaaacataattgaatacagcagcaattggcaagctgcttacatagaactc |
| | gcggcgattggcatgccgccttaaaatttttattttatttttcttttcttttccgaatcggattttgttttta |
| | atatttcaaaaaaaaaaaaaaaaaaaaaaaaaaaaaaaaaaaaaaaaaaaaaaaaaaaaaaaaaaaaaaaaaa |
| | gaagagctagggataacagggtaattgagcaaaaggccagcaaaaggccaggaaccgtaaaaaggccgcgttg |
| | ctggcgtttttccataggctccgcccccctgacgagcatcacaaaaatcgacgctcaagtcagaggtggcgaa |
| | acccgacaggactataaagataccaggcgtttccccctggaagctccctcgtgcgctctcctgttccgaccct |
| | gccgcttaccggatacctgtccgcctttctcccttcgggaagcgtggcgctttctcatagctcacgctgtagg |
| | tatctcagttcggtgtaggtcgttcgctccaagctgggctgtgtgcacgaaccccccgttcagcccgaccgct |
| | gcgccttatccggtaactatcgtcttgagtccaacccggtaagacacgacttatcgccactggcagcagccac |
| | tggtaacaggattagcagagcgaggtatgtaggcggtgctacagagttcttgaagtggtggcctaactacggc |
| | tacactagaagaacagtatttggtatctgcgctctgctgaagccagttaccttcggaaaaagagttggtagct |
| | cttgatccggcaaacaaaccaccgctggtagcggtggtttttttgtttgcaagcagcagattacgcgcagaaa |
| | aaaaggatctcaagaagatcctttgatcttttctacggggtctgacgctcagtggaacgaaaactcacgtta |
| | agggattttggtcatgagattatcaaaaaggatcttcacctagatccttttaaattaaaaatgaagttttaaa |
| | tcaatctaaagtatatatgagtaaacttggtctgacagttagaaaaactcatcgagcatcaaatgaaactgca |
| | atttattcatatcaggattatcaataccatatttttgaaaaagccgtttctgtaatgaaggagaaaactcac |
| | cgaggcagttccataggatggcaagatcctggtatcggtctgcgattccgactcgtccaacatcaatacaacc |
| | tattaatttcccctcgtcaaaaataaggttatcaagtgagaaatcaccatgagtgacgactgaatccggtgag |
| | aatggcaaaagtttatgcatttctttccagacttgttcaacaggccagccattacgctcgtcatcaaaatca |
| | ctcgcatcaaccaaaccgttattcattcgtgattgcgcctgagcgagacgaaatacgcgatcgctgttaaaa |
| | ggacaattacaaacaggaatcgaatgcaaccggcgcaggaacactgccagcgcatcaacaatattttcacctg |
| | aatcaggatattcttctaatacctggaatgctgttttcccagggatcgcagtggtgagtaaccatgcatcat |
| | caggagtacggataaaatgcttgatggtcggaagaggcataaattccgtcagccagtttagtctgaccatct |
| | catctgtaacatcattggcaacgctacctttgccatgtttcagaaacaactctggcgcatcgggcttcccata |
| | caatcgatagattgtcgcacctgattgcccgacattatcgcgagcccatttatacccatataaatcagcatc |
| | catgttggaatttaatcgcggcctagagcaagacgtttcccgttgaatatggctcatactcttcctttttcaa |
| | tattattgaagcatttatcagggttattgtctcatgagcggatacatatttgaatgtatttagaaaaataaac |
| | aaataggggttccgcgcacatttccccgaaaagtgccacctgacgttaatacgactcactatagg |

1. A composition comprising:
a) a self-replicating RNA (srRNA) comprising at least one nucleotide sequence encoding interleukin-12 (IL-12) comprising p40 and p35; and
b) a lipid nanoparticle (LNP) comprising an ionizable lipid having the Formula:

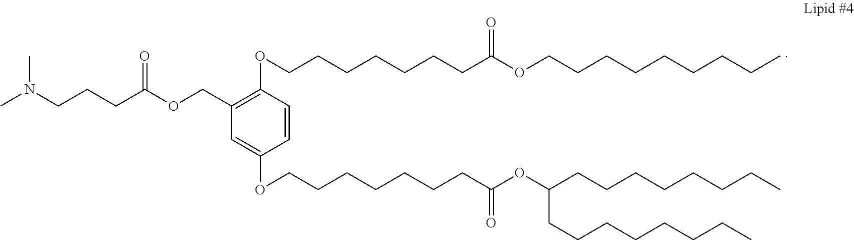 [SMILES: CCCCCCCCCOC(=O)CCCCCCCOc1ccc(OCCCCCCCC(=O)OC(CCCCCCCC)CCCCCCCC)cc1COC(=O)CCCN(C)C]

2. The composition of claim 1, wherein the srRNA comprises a nucleic acid sequence having at least 80% sequence identity to the nucleic acid sequence of SEQ ID NO: 10 or 11.

3.-4. (canceled)

5. The composition of claim 1, wherein p40 and p35 are operably linked, directly linked, or lined via a cleavable linker.

6.-7. (canceled)

8. The composition of claim 1, wherein the interleukin-12 comprises human interleukin-12 and/or human p40 and human p35.

9.-12. (canceled)

13. The composition of claim 8, wherein interleukin-12 comprises the sequence shown in SEQ ID NO: 1.

14. (canceled)

15. The composition of claim 8, wherein
(i) the sequence of p35 comprises the sequence shown in SEQ ID NO: 3;
(ii) the sequence of p40 comprises the sequence shown in SEQ ID NO: 2; and/or
(iii) the sequence of the linker comprises the sequence shown in SEQ ID NO: 4.

16.-20. (canceled)

21. The composition of claim 8, wherein the nucleotide sequence encoding interleukin-12 is operably linked to a promoter, optionally a subgenomic promoter, and/or the nucleotide sequence encoding p40 is operably linked to the promoter.

22. The composition of claim 1, wherein the nucleotide sequence encoding interleukin-12 comprises, from 5′ to 3′, a nucleotide sequence encoding p40, a nucleotide sequence encoding a linker, and nucleotide sequence encoding p35, and optionally wherein the nucleotide sequence encoding interleukin-12 is linked to a promoter located 5′ relative to the nucleotide sequence encoding p40.

23. The composition of claim 1, wherein the srRNA comprises
(i) a 5′ cap untranslated region (UTR), one or more non-structural genes, a promoter, and a 3′ terminal polyadenylated (polyA) region; or
(ii) from 5′ to 3′, a 5′ UTR, one or more non-structural genes, a promoter, the nucleotide sequence encoding interleukin-12, and a 3′ polyA region.

24.-27. (canceled)

28. The composition of claim 23, wherein the srRNA lacks one or more nucleotide sequences encoding one or more structural protein sequences, optionally wherein the nucleotide sequence encoding interleukin-12 is inserted in place of the one or more nucleotide sequences encoding the one or more structural protein sequences.

29. The composition of claim 1, wherein the srRNA is a TC-83 VEEV srRNA.

30.-34. (canceled)

35. The composition of claim 1, wherein the LNP comprises
(i) a cationic ionizable cationic lipid, 1,2-Diastearoyl-sn-glycero-3-phosphocholine (DSPC), Cholesterol, and 1,2-dimyristoyl-sn-glycero-3-phosphoethanolamine-N-[methoxy(polyethylene glycol)-2000] (DMG-PEG2000);
(ii) a ionizable cationic lipid, 1,2-Dioleoyl-sn-glycero-3-phosphoethanolamine (DOPE), Cholesterol, and 1,2-dimyristoyl-sn-glycero-3-phosphoethanolamine-N-[methoxy(polyethylene glycol)-2000] (DMG-PEG2000); or
(iii) an ionizable cationic lipid, 1,2-Dioleoyl-sn-glycero-3-phosphoethanolamine (DOPE), Cholesterol, and a pegylated lipid comprising a polyethylene glycol moiety.

36.-38. (canceled)

39. The composition of claim 1, wherein the composition comprises
(i) a DSPC:cholesterol:PEG:ionizable lipid mole ratio ranging from 5:20:0.5:20 to 25:70:5:60, optionally 10:48:2:40, at a N:P (lipid:srRNA) ratio ranging from 2:1 to 12:1, optionally 8:1; or
(ii) a DOPE:cholesterol:PEG:ionizable lipid mole ratio ranging from 5:20:0:20 to 25:70:5:60, optionally 10:48:2:40, at a N:P (lipid:srRNA) ratio ranging from 2:1 to 12:1, optionally 8:1;
wherein the composition has a particle size of about 40 to about 300 nanometer (nm).

40.-41. (canceled)

42. The composition of claim 1, wherein the composition enhances an immune response in a subject following administration.

43. The composition of claim 42, wherein the immune response comprises an antitumor immune response, wherein the immune response comprises T cells and/or CD8+ cells.

44. (canceled)

45. The composition of claim 1, wherein the composition lacks a separate adjuvant component.

46. A self-replicating messenger RNA (srRNA) comprising at least one nucleotide sequence encoding interleukin-12 (IL-12) comprising p40 and p35 comprising the nucleic acid sequence of SEQ ID NO: 11.

47.-79. (canceled)

80. A method of enhancing an immune response in a subject, comprising administering the srRNA of claim 46 or the composition of claim 1 to the subject.

81. A method of treating a tumor in a subject, comprising administering the srRNA of claim 46 or the composition of claim 1 to the subject.

82. The method of claim 80, wherein the composition is administered to the subject
(i) intratumorally, intramuscularly, or intravenously;
(ii) at least three times;
(iii) once every one, two, three or four weeks;
(iv) at a dose of 0.1-200 μg; and/or
(iv) further comprises the administration of a checkpoint inhibitor, optionally wherein the checkpoint inhibitor is administered prior to, concurrent with, or following administration of the srRNA or the composition.

83.-89. (canceled)
